SEQUENCE LISTING (33 sequences)

SEQ ID NO: 1 - DNA, 39 bp, artificial
  primer sequence
cagcgacatatgtcgctgaccgcatgccctgaggagtcc

SEQ ID NO: 2 - DNA, 36 bp, artificial
  primer sequence
gacacctccaaagttctgcacgtaaggtaggctaaa

SEQ ID NO: 3 - DNA, 39 bp, artificial
  primer sequence
ctgatggatggatccctagctcggcgtcccgatgtccac

SEQ ID NO: 4 - DNA, 36 bp, artificial
  primer sequence
ccttacgtgcagaactttggaggtgtctctgctcta

SEQ ID NO: 5 - DNA, 36 bp, artificial
  primer sequence
gacacctccaaaaaactgcacgtaaggtaggctaaa

SEQ ID NO: 6 - DNA, 36 bp, artificial
  primer sequence
ccttacgtgcagttttttggaggtgtctctgctcta

SEQ ID NO: 7 - DNA, 36 bp, artificial
  primer sequence
gacacctccaaacatctgcacgtaaggtaggctaaa

SEQ ID NO: 8 - DNA, 36 bp, artificial
  primer sequence
ccttacgtgcagatgtttggaggtgtctctgctcta

SEQ ID NO: 9 - DNA, 36 bp, artificial
  primer sequence
gacacctccaaaaatctgcacgtaaggtaggctaaa

SEQ ID NO: 10 - DNA, 36 bp, artificial
  primer sequence
ccttacgtgcagatttttggaggtgtctctgctcta

SEQ ID NO: 11 - DNA, 36 bp, artificial
  primer sequence
gacacctccaaacacctgcacgtaaggtaggctaaa

SEQ ID NO: 12 - DNA, 36 bp, artificial
  primer sequence
ccttacgtgcaggtgtttggaggtgtctctgctcta

SEQ ID NO: 13 - DNA, 36 bp, artificial
  primer sequence
gacacctccaaacgcctgcacgtaaggtaggctaaa

SEQ ID NO: 14 - DNA, 36 bp, artificial
  primer sequence
ccttacgtgcaggcgtttggaggtgtctctgctcta

SEQ ID NO: 15 - DNA, 36 bp, artificial
  primer sequence
gacacctccaaagccctgcacgtaaggtaggctaaa

SEQ ID NO: 16 - DNA, 36 bp, artificial
  primer sequence
ccttacgtgcagggctttggaggtgtctctgctcta

SEQ ID NO: 17 - protein, 402 aa, Bos taurus
MKFREPLLGGSAAMPGASLQRACRLLVAVCALHLGVTLVYYLAGRDLRRLPQLVGVHPPLQGSSHGAAAIGQPSGELRLRGVAPPPPLQNSSKPRSRAPSNLDAYSHPGPGPGPGSNLTSAPVPSTTTRSLTACPEESPLLVGPMLIEFNIPVDLKLIEQQNPKVKLGGRYTPMDCISPHKVAIIILFRNRQEHLKYWLYYLHPMVQRQQLDYGIYVINQAGESMFNRAKLLNVGFKEALKDYDYNCFVFSDVDLIPMNDHNTYRCFSQPRHISVAMDKFGFSLPYVQYFGGVSALSKQQFLSINGFPNNYWGWGGEDDDIYNRLAFRGMSVSRPNAVIGKCRMIRHSRDKKNEPNPQRFDRIAHTKETMLSDGLNSLTYMVLEVQRYPLYTKITVDIGTPS

SEQ ID NO: 18 - protein, 402 aa, artificial
  bos taurus GalT Y289N
MKFREPLLGGSAAMPGASLQRACRLLVAVCALHLGVTLVYYLAGRDLRRLPQLVGVHPPLQGSSHGAAAIGQPSGELRLRGVAPPPPLQNSSKPRSRAPSNLDAYSHPGPGPGPGSNLTSAPVPSTTTRSLTACPEESPLLVGPMLIEFNIPVDLKLIEQQNPKVKLGGRYTPMDCISPHKVAIIILFRNRQEHLKYWLYYLHPMVQRQQLDYGIYVINQAGESMFNRAKLLNVGFKEALKDYDYNCFVFSDVDLIPMNDHNTYRCFSQPRHISVAMDKFGFSLPYVQNFGGVSALSKQQFLSINGFPNNYWGWGGEDDDIYNRLAFRGMSVSRPNAVIGKCRMIRHSRDKKNEPNPQRFDRIAHTKETMLSDGLNSLTYMVLEVQRYPLYTKITVDIGTPS

SEQ ID NO: 19 - protein, 402 aa, artificial
  bos taurus GalT Y289F
MKFREPLLGGSAAMPGASLQRACRLLVAVCALHLGVTLVYYLAGRDLRRLPQLVGVHPPLQGSSHGAAAIGQPSGELRLRGVAPPPPLQNSSKPRSRAPSNLDAYSHPGPGPGPGSNLTSAPVPSTTTRSLTACPEESPLLVGPMLIEFNIPVDLKLIEQQNPKVKLGGRYTPMDCISPHKVAIIILFRNRQEHLKYWLYYLHPMVQRQQLDYGIYVINQAGESMFNRAKLLNVGFKEALKDYDYNCFVFSDVDLIPMNDHNTYRCFSQPRHISVAMDKFGFSLPYVQFFGGVSALSKQQFLSINGFPNNYWGWGGEDDDIYNRLAFRGMSVSRPNAVIGKCRMIRHSRDKKNEPNPQRFDRIAHTKETMLSDGLNSLTYMVLEVQRYPLYTKITVDIGTPS

SEQ ID NO: 20 - protein, 402 aa, artificial
  bos taurus GalT Y289M
MKFREPLLGGSAAMPGASLQRACRLLVAVCALHLGVTLVYYLAGRDLRRLPQLVGVHPPLQGSSHGAAAIGQPSGELRLRGVAPPPPLQNSSKPRSRAPSNLDAYSHPGPGPGPGSNLTSAPVPSTTTRSLTACPEESPLLVGPMLIEFNIPVDLKLIEQQNPKVKLGGRYTPMDCISPHKVAIIILFRNRQEHLKYWLYYLHPMVQRQQLDYGIYVINQAGESMFNRAKLLNVGFKEALKDYDYNCFVFSDVDLIPMNDHNTYRCFSQPRHISVAMDKFGFSLPYVQMFGGVSALSKQQFLSINGFPNNYWGWGGEDDDIYNRLAFRGMSVSRPNAVIGKCRMIRHSRDKKNEPNPQRFDRIAHTKETMLSDGLNSLTYMVLEVQRYPLYTKITVDIGTPS

SEQ ID NO: 21 - protein, 402 aa, artificial
  bos taurus GalT Y289I
MKFREPLLGGSAAMPGASLQRACRLLVAVCALHLGVTLVYYLAGRDLRRLPQLVGVHPPLQGSSHGAAAIGQPSGELRLRGVAPPPPLQNSSKPRSRAPSNLDAYSHPGPGPGPGSNLTSAPVPSTTTRSLTACPEESPLLVGPMLIEFNIPVDLKLIEQQNPKVKLGGRYTPMDCISPHKVAIIILFRNRQEHLKYWLYYLHPMVQRQQLDYGIYVINQAGESMFNRAKLLNVGFKEALKDYDYNCFVFSDVDLIPMNDHNTYRCFSQPRHISVAMDKFGFSLPYVQIFGGVSALSKQQFLSINGFPNNYWGWGGEDDDIYNRLAFRGMSVSRPNAVIGKCRMIRHSRDKKNEPNPQRFDRIAHTKETMLSDGLNSLTYMVLEVQRYPLYTKITVDIGTPS

SEQ ID NO: 22 - protein, 402 aa, artificial
  bos taurus GalT Y289V
MKFREPLLGGSAAMPGASLQRACRLLVAVCALHLGVTLVYYLAGRDLRRLPQLVGVHPPLQGSSHGAAAIGQPSGELRLRGVAPPPPLQNSSKPRSRAPSNLDAYSHPGPGPGPGSNLTSAPVPSTTTRSLTACPEESPLLVGPMLIEFNIPVDLKLIEQQNPKVKLGGRYTPMDCISPHKVAIIILFRNRQEHLKYWLYYLHPMVQRQQLDYGIYVINQAGESMFNRAKLLNVGFKEALKDYDYNCFVFSDVDLIPMNDHNTYRCFSQPRHISVAMDKFGFSLPYVQVFGGVSALSKQQFLSINGFPNNYWGWGGEDDDIYNRLAFRGMSVSRPNAVIGKCRMIRHSRDKKNEPNPQRFDRIAHTKETMLSDGLNSLTYMVLEVQRYPLYTKITVDIGTPS

SEQ ID NO: 23 - protein, 402 aa, artificial
  bos taurus GalT Y289A
MKFREPLLGGSAAMPGASLQRACRLLVAVCALHLGVTLVYYLAGRDLRRLPQLVGVHPPLQGSSHGAAAIGQPSGELRLRGVAPPPPLQNSSKPRSRAPSNLDAYSHPGPGPGPGSNLTSAPVPSTTTRSLTACPEESPLLVGPMLIEFNIPVDLKLIEQQNPKVKLGGRYTPMDCISPHKVAIIILFRNRQEHLKYWLYYLHPMVQRQQLDYGIYVINQAGESMFNRAKLLNVGFKEALKDYDYNCFVFSDVDLIPMNDHNTYRCFSQPRHISVAMDKFGFSLPYVQAFGGVSALSKQQFLSINGFPNNYWGWGGEDDDIYNRLAFRGMSVSRPNAVIGKCRMIRHSRDKKNEPNPQRFDRIAHTKETMLSDGLNSLTYMVLEVQRYPLYTKITVDIGTPS

SEQ ID NO: 24 - protein, 402 aa, artificial
  bos taurus GalT Y289G
MKFREPLLGGSAAMPGASLQRACRLLVAVCALHLGVTLVYYLAGRDLRRLPQLVGVHPPLQGSSHGAAAIGQPSGELRLRGVAPPPPLQNSSKPRSRAPSNLDAYSHPGPGPGPGSNLTSAPVPSTTTRSLTACPEESPLLVGPMLIEFNIPVDLKLIEQQNPKVKLGGRYTPMDCISPHKVAIIILFRNRQEHLKYWLYYLHPMVQRQQLDYGIYVINQAGESMFNRAKLLNVGFKEALKDYDYNCFVFSDVDLIPMNDHNTYRCFSQPRHISVAMDKFGFSLPYVQGFGGVSALSKQQFLSINGFPNNYWGWGGEDDDIYNRLAFRGMSVSRPNAVIGKCRMIRHSRDKKNEPNPQRFDRIAHTKETMLSDGLNSLTYMVLEVQRYPLYTKITVDIGTPS

SEQ ID NO: 25 - protein, 273 aa, Artificial
  Gal-T1 130-402 (Y289L,C342T)
SLTACPEESPLLVGPMLIEFNIPVDLKLIEQQNPKVKLGGRYTPMDCISPHKVAIIILFRNRQEHLKYWLYYLHPMVQRQQLDYGIYVINQAGESMFNRAKLLNVGFKEALKDYDYNCFVFSDVDLIPMNDHNTYRCFSQPRHISVAMDKFGFSLPYVQLFGGVSALSKQQFLSINGFPNNYWGWGGEDDDIYNRLAFRGMSVSRPNAVIGKTRMIRHSRDKKNEPNPQRFDRIAHTKETMLSDGLNSLTYMVLEVQRYPLYTKITVDIGTPS

SEQ ID NO: 26 - protein, 329 aa, Artificial
  Gal-T1 74-402 (Y289L)
SGELRLRGVAPPPPLQNSSKPRSRAPSNLDAYSHPGPGPGPGSNLTSAPVPSTTTRSLTACPEESPLLVGPMLIEFNIPVDLKLIEQQNPKVKLGGRYTPMDCISPHKVAIIILFRNRQEHLKYWLYYLHPMVQRQQLDYGIYVINQAGESMFNRAKLLNVGFKEALKDYDYNCFVFSDVDLIPMNDHNTYRCFSQPRHISVAMDKFGFSLPYVQLFGGVSALSKQQFLSINGFPNNYWGWGGEDDDIYNRLAFRGMSVSRPNAVIGKCRMIRHSRDKKNEPNPQRFDRIAHTKETMLSDGLNSLTYMVLEVQRYPLYTKITVDIGTPS

SEQ ID NO: 27 - protein, 329 aa, Artificial
  Gal-T1 74-402 (Y289F)
SGELRLRGVAPPPPLQNSSKPRSRAPSNLDAYSHPGPGPGPGSNLTSAPVPSTTTRSLTACPEESPLLVGPMLIEFNIPVDLKLIEQQNPKVKLGGRYTPMDCISPHKVAIIILFRNRQEHLKYWLYYLHPMVQRQQLDYGIYVINQAGESMFNRAKLLNVGFKEALKDYDYNCFVFSDVDLIPMNDHNTYRCFSQPRHISVAMDKFGFSLPYVQFFGGVSALSKQQFLSINGFPNNYWGWGGEDDDIYNRLAFRGMSVSRPNAVIGKCRMIRHSRDKKNEPNPQRFDRIAHTKETMLSDGLNSLTYMVLEVQRYPLYTKITVDIGTPS

SEQ ID NO: 28 - protein, 329 aa, Artificial
  Gal-T1 74-402 (Y289M)
SGELRLRGVAPPPPLQNSSKPRSRAPSNLDAYSHPGPGPGPGSNLTSAPVPSTTTRSLTACPEESPLLVGPMLIEFNIPVDLKLIEQQNPKVKLGGRYTPMDCISPHKVAIIILFRNRQEHLKYWLYYLHPMVQRQQLDYGIYVINQAGESMFNRAKLLNVGFKEALKDYDYNCFVFSDVDLIPMNDHNTYRCFSQPRHISVAMDKFGFSLPYVQMFGGVSALSKQQFLSINGFPNNYWGWGGEDDDIYNRLAFRGMSVSRPNAVIGKCRMIRHSRDKKNEPNPQRFDRIAHTKETMLSDGLNSLTYMVLEVQRYPLYTKITVDIGTPS

SEQ ID NO: 29 - protein, 329 aa, Artificial
  Gal-T1 74-402 (Y289V)
SGELRLRGVAPPPPLQNSSKPRSRAPSNLDAYSHPGPGPGPGSNLTSAPVPSTTTRSLTACPEESPLLVGPMLIEFNIPVDLKLIEQQNPKVKLGGRYTPMDCISPHKVAIIILFRNRQEHLKYWLYYLHPMVQRQQLDYGIYVINQAGESMFNRAKLLNVGFKEALKDYDYNCFVFSDVDLIPMNDHNTYRCFSQPRHISVAMDKFGFSLPYVQVFGGVSALSKQQFLSINGFPNNYWGWGGEDDDIYNRLAFRGMSVSRPNAVIGKCRMIRHSRDKKNEPNPQRFDRIAHTKETMLSDGLNSLTYMVLEVQRYPLYTKITVDIGTPS

SEQ ID NO: 30 - protein, 329 aa, Artificial
  Gal-T1 74-402 (Y289A)
SGELRLRGVAPPPPLQNSSKPRSRAPSNLDAYSHPGPGPGPGSNLTSAPVPSTTTRSLTACPEESPLLVGPMLIEFNIPVDLKLIEQQNPKVKLGGRYTPMDCISPHKVAIIILFRNRQEHLKYWLYYLHPMVQRQQLDYGIYVINQAGESMFNRAKLLNVGFKEALKDYDYNCFVFSDVDLIPMNDHNTYRCFSQPRHISVAMDKFGFSLPYVQAFGGVSALSKQQFLSINGFPNNYWGWGGEDDDIYNRLAFRGMSVSRPNAVIGKCRMIRHSRDKKNEPNPQRFDRIAHTKETMLSDGLNSLTYMVLEVQRYPLYTKITVDIGTPS

SEQ ID NO: 31 - protein, 329 aa, Artificial
  Gal-T1 74-402 (Y289G)
SGELRLRGVAPPPPLQNSSKPRSRAPSNLDAYSHPGPGPGPGSNLTSAPVPSTTTRSLTACPEESPLLVGPMLIEFNIPVDLKLIEQQNPKVKLGGRYTPMDCISPHKVAIIILFRNRQEHLKYWLYYLHPMVQRQQLDYGIYVINQAGESMFNRAKLLNVGFKEALKDYDYNCFVFSDVDLIPMNDHNTYRCFSQPRHISVAMDKFGFSLPYVQGFGGVSALSKQQFLSINGFPNNYWGWGGEDDDIYNRLAFRGMSVSRPNAVIGKCRMIRHSRDKKNEPNPQRFDRIAHTKETMLSDGLNSLTYMVLEVQRYPLYTKITVDIGTPS

SEQ ID NO: 32 - protein, 329 aa, Artificial
  Gal-T1 74-402 (Y289L C342T)
SGELRLRGVAPPPPLQNSSKPRSRAPSNLDAYSHPGPGPGPGSNLTSAPVPSTTTRSLTACPEESPLLVGPMLIEFNIPVDLKLIEQQNPKVKLGGRYTPMDCISPHKVAIIILFRNRQEHLKYWLYYLHPMVQRQQLDYGIYVINQAGESMFNRAKLLNVGFKEALKDYDYNCFVFSDVDLIPMNDHNTYRCFSQPRHISVAMDKFGFSLPYVQLFGGVSALSKQQFLSINGFPNNYWGWGGEDDDIYNRLAFRGMSVSRPNAVIGKTRMIRHSRDKKNEPNPQRFDRIAHTKETMLSDGLNSLTYMVLEVQRYPLYTKITVDIGTPS

SEQ ID NO: 33 - protein, 329 aa, Artificial
  Gal-T1 74-402 (Y289M C342T)
SGELRLRGVAPPPPLQNSSKPRSRAPSNLDAYSHPGPGPGPGSNLTSAPVPSTTTRSLTACPEESPLLVGPMLIEFNIPVDLKLIEQQNPKVKLGGRYTPMDCISPHKVAIIILFRNRQEHLKYWLYYLHPMVQRQQLDYGIYVINQAGESMFNRAKLLNVGFKEALKDYDYNCFVFSDVDLIPMNDHNTYRCFSQPRHISVAMDKFGFSLPYVQMFGGVSALSKQQFLSINGFPNNYWGWGGEDDDIYNRLAFRGMSVSRPNAVIGKTRMIRHSRDKKNEPNPQRFDRIAHTKETMLSDGLNSLTYMVLEVQRYPLYTKITVDIGTPS

(19) United States
(12) Patent Application Publication
(10) Pub. No.: US 2017/0009266 A1
(43) Pub. Date: Jan. 12, 2017

(54) PROCESS FOR THE ATTACHMENT OF A GALNAC MOIETY COMPRISING A (HETERO)ARYL GROUP TO A GLCNAC MOIETY, AND PRODUCT OBTAINED THEREBY

(71) Applicant: SYNAFFIX B.V., Oss (NL)

(72) Inventors: Floris Louis VAN DELFT, Nijmegen (NL); Remon VAN GEEL, Lith-Oijen (NL); Maria Antonia WIJDEVEN, Lent (NL); Ryan HEESBEEN, Nijmegen (NL)

(73) Assignee: SynAffix B.V., Oss, (NL)

(21) Appl. No.: 15/113,740

(22) PCT Filed: Jan. 26, 2015
(86) PCT No.: PCT/NL2015/050044

§ 371 Date: Jul. 22, 2016

(30) Foreign Application Priority Data
Jan. 24, 2014 (EP) 14152434.8
Jan. 24, 2014 (EP) 14152500.6

(51) Int. Cl. C12P 19/18; C07K 16/32; C12P 19/30; C07K 16/24; C12P 21/00; C07H 19/06; C07K 16/28

(52) U.S. Cl. CPC C12P 19/18; C07H 19/06; C07K 16/32; C07K 16/2863; C07K 16/241; C12P 21/005; C12P 19/305; C12Y 204/01; C07K 2317/41; C07K 2317/24; C07K 2317/21

(57) ABSTRACT

The present invention relates to a process for attaching an N-acetylgalactosamine-(hetero)arylmoiety to an N-acetylglucosaminemoiety, the process comprising the step of contacting the N-acetylgalactosamine-(hetero)arylmoiety with the N-acetylglucosaminemoiety in the presence of a mutant galactosyltransferase, wherein the N-acetylglucosaminemoiety is according to Formula (1) the N-acetylgalactosamine-(hetero)arylmoiety is according to Formula (2): In a particularly preferred embodiment of the process according to the invention, the N-acetylgalactosamine-(hetero)arylmoiety comprises a 1,3-dipole functional group, and the N-acetylglucosaminemoiety is a terminal GlcNAc moiety of a glycoprotein glycan. The invention further relates to a product obtainable by the process according to the invention, in particular to glycoproteins. Also, the invention relates to several compounds comprising an N-acetylgalactosamine-(hetero)arylmoiety.

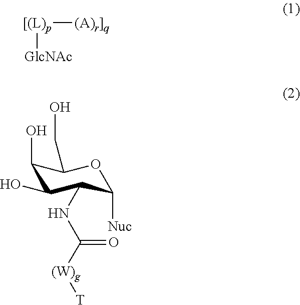

### TECHNICAL FIELD OF THE INVENTION

The invention relates to a process for the attachment of an N-acetylgalactosamine moiety comprising a (hetero)aryl group to an N-acetylglucosamine moiety, in the presence of a mutant galactosyltransferase. The N-acetylglucosamine moiety may be comprised in a glycoprotein glycan. The invention therefore also relates to glycoproteins wherein a glycan comprises a terminal N-acetylgalactosamine moiety substituted with a (hetero)aryl group.

### BACKGROUND OF THE INVENTION

Glycosylation of biomolecules including natural products, proteins and lipids mediates a wide variety of important biological processes. It is well established that the carbohydrate portions of these molecules are essential for bioactivity as there exist many cases where deglycosylated versions show little or no bioactivity compared to their glycosylated counterparts. Although the precise role of the sugar residue varies, carbohydrates have traditionally been implicated in specific interactions with biological targets as well as in drug pharmacokinetics.

Access to single glycoforms of such glycosylated compounds can be achieved by a large variety of organic chemical tools. However, such synthetic approaches are arduous, and do not realistically represent a practical approach that can normally be applied to widespread and large-scale production. The latter pertains in particular to the field of glycopeptides and glycoproteins, the synthesis of which is further enhanced by the sensitivity and lack of compatibility of the (poly)peptide fragment of the molecule with standard chemical techniques.

An alternative to organic synthesis for the preparation of homogeneous glycoforms of a particular substance involves the use of enzymatic catalysis. Thus, the treatment of a sugar (carbohydrate fragment) with a specific enzyme/substrate combination is a powerful method for highly controlled, regioselective modification under aqueous conditions. In vivo glycosylation is mediated by Leloir-type glycosyltransferase enzymes, which are among the most abundant enzymes in Nature. In vivo, glycosyltransferases have high specificity in transferring a sugar from a nucleotide donor to an acceptor substrate to form glycosidic linkages. However, for glycosyltransferase-mediated reactions that are performed in vitro, the enzymes typically tolerate a somewhat broader set of substrates and have therefore been useful catalysts in the synthesis of oligosaccharides and derivatives. The second class of enzymes that display considerable synthetic potential are the endohexosaminidases. While normally aimed to cleave the chitobiose core [GlcNAc(1-4)GlcNAc] of N-linked glycans between the two N-acetyl glucosamine residues by hydrolysis, specific mutation strategies enable the possibility to use the same enzyme to effectively synthesize glycosidic bonds instead. Whichever strategy employed, in general the use of enzymes to synthesize complex oligosaccharides offers the benefit that defined glycosidic linkages are created with high efficiency at neutral pH, and tedious protection and deprotection steps that are required in organic synthesis are avoided.

Many of the natural glycosyltransferases reside in the Golgi apparatus of a cell, where the oligosaccharide chains is synthesized by transferring a monosaccharide moiety from an activated sugar donor to an acceptor molecule, forming a glycosidic bond. Glycosyltransferases are named after the sugar moiety that is transferred and are further divided into subfamilies, based on the linkage generated between the donor and acceptor. The galactosyltransferase family, in the presence of metal ion, transfers galactose from uridine-diphosphate-α-D-galactose (UDP-Gal) to an acceptor sugar molecule (FIG. 1).

After the first glycosyltransferase, β(1,4)-galactosyltransferase (β4Gal-T) was cloned, subfamilies of inverting galactosyltransferases, β(1,4)-(β4Gal-T), β(1,3)-(β3Gal-T), and β(1,6)-(β6Gal-T), and retaining galactosyltransferases, α(1,3)-(α3-Gal-T) and α(1,4)-(α4Gal-T), have been identified. All of them use UDP-α-D-Gal as the sugar donor but generate β(1,4)-, β(1,3)-, β(1,6)-, α(1,3)- and α(1,4)-linkages, respectively (FIG. 1). Each subfamily has additional members. The β4Gal-T subfamily consists of at least seven members, Gal-T1 to Gal-T7, with a 25% to 55% sequence homology. Each subfamily member is expressed in a tissue-specific manner and shows differences in the oligosaccharide acceptor specificity. Among β4Gal-T subfamily members, β4Gal-T1 interacts with α-lactalbumin (LA), a protein expressed in the mammary gland during lactation, to form the lactose synthase (LS) complex that transfers galactose from UDP-α-D-Gal to glucose, producing the lactose secreted in milk.

The sugar donor specificity of glycosyltransferases is generally determined by a few crucial residues in the binding pocket since mutation of these residues broadens the donor specificity. Nevertheless, it has been demonstrated on several occasions that the native GalT enzyme can also employed for the galactosylation of GlcNAc acceptor substrates with derivatives of galactose, modified specifically at C-6. For example, Elling et al. in ChemBioChem 2001, 2, 884, incorporated by reference, have shown that a 6-biotinylated version of UDP-galactose (13a, FIG. 2) can be enzymatically transferred to GlcNAc-4-methylumbelliferin upon incubation with different galactosyl transferases (FIG. 3, top). Similarly, WO 2006/035057 (Novo Nordisk A/S), incorporated by reference herein, demonstrated that a range of other 6-modified UDP-galactose derivatives can be transferred to GlcNAc acceptor substrates. Finally, Pannecoucke et al. in Tetrahedron Lett. 2008, 49, 2294, incorporated by reference, have also shown that UDP-6-azidogalactose (13b, FIG. 2) can be enzymatically transferred to GlcNAc-4-methylumbelliferin upon incubation with native β4Gal-T1 (FIG. 3, top).

In bovine β4Gal-T1, the specificity toward the nucleotide sugar, UDP-Gal, is determined by a tyrosine (or phenylalanine) residue at position 289 in the binding pocket. The residue Tyr or Phe is highly conserved among family members from different species at the corresponding position. The β4Gal-T1 transfers GalNAc sugar moiety from the sugar donor UDP-GalNAc to an acceptor at only 0.1% efficiency compared to Gal transfer from UDP-Gal. This poor transfer of GalNAc from UDP-GalNAc is due to the Tyr residue in the catalytic pocket of β4Gal-T1, which restricts this transfer by forming a hydrogen bond with the N-acetyl group of GalNAc. Thus, Tyr289 acts as a molecular brake on the GalNAc moiety and restricts its transfer from UDP-GalNAc to the acceptor molecule.

Qasba et al. disclose in J. Biol. Chem. 2002, 277, 20833, incorporated by reference herein, that mutant galactosyltransferases GalT(Y289L), GalT(Y289I) and GalT(Y289N) can enzymatically attach GalNAc to a non-reducing GlcNAc sugar (β-benzyl-GlcNAc) (FIG. 3, bottom). By substituting Tyr289 for Leu, Asn or Ile, the molecular brake restriction is removed and the mutants Y289L, Y289N or Y289I were all able to transfer GalNAc to a GlcNAc acceptor, of which β4Gal-T1(Y289L)/GalNAc with nearly 100% of the efficiency of the β4Gal-T1/Gal transfer. In later years, it was demonstrated that this particular Y289L mutant was also able to transfer unnatural UDP-Gal C2 analogues. Synthetic variants of UDP-GalNAc (FIG. 3) that have been used as substrates for β4Gal-T1(Y289L) include a 2′-keto derivative of galactose (C2-keto-Gal, 15, FIG. 2) or N-azidoacetylgalactosamine (GalNAz, 16, FIG. 2). For example, WO 2007/095506 and WO 2008/029281 (Invitrogen Corporation), both incorporated by reference herein, disclose that the combination of GalT(Y289L) mutant with the C2-substituted azidoacetamido moiety 2-GalNAz-UDP leads to the incorporation of GalNAz at a terminal non-reducing GlcNAc of a glycan (FIG. 3, bottom).

Glycoproteins can be site-specifically conjugated by application of the β4Gal-T1(Y289L) mutant in combination with an unnatural sugar. For example, enzymatic transfer of an unnatural substrate to the non-reducing end of the glycan of the glycoprotein installs a chemical handle suitable for subsequent site-specific conjugation with biologically important molecules having a corresponding orthogonal chemical group. For example, it has been described by Hsieh-Wilson and coworkers that β4Gal-T1(Y289L) can be applied for in vitro detection of O-GlcNAc residues on proteins (FIG. 4, left), for selectively biotinylation of proteins with posttranslational O-GlcNAc modifications and then identify them using a horseradish peroxidase-based chemiluminescence reporter system. In more recent work, it has been shown that the biantennary N-glycans of a therapeutic IgG molecule can be used for conjugation with bioactive molecules such as biotin or fluorescent moieties to both arms of the biantenary N-glycans, thus producing the native IgG molecule with four biotin molecules site-specifically (FIG. 5, middle). In 2008, Clark, et al. used the azide-bearing UDP-N-acetylgalactosamine analog UDP-GalNAz and an alkyne-modified fluorescent reporter to create a system for the detection, proteomic analysis, and cellular imaging of O-GlcNAc-modified proteins using canonical Cu-catalyzed azide-alkyne (3+2) cycloaddition click chemistry.

Qasba et al. disclose in Bioconjugate Chem. 2009, 20, 1228, incorporated by reference herein, that β-galactosidase-treated monoclonal antibodies (e.g. Rituxan, Remicade, Herceptin) having a G0 glycoform (obtained by treatment of the crude mAbs with galactosidase) are fully re-galactosylated to the G2 glycoform after transfer of an oligosaccharide comprising a galactose moiety comprising an azide group to the terminal GlcNAc residues of the glycan, leading to tetraazido-substituted antibodies, i.e. two GalNAz moieties per heavy chain. The conjugation of said tetraazido-substituted antibodies to a molecule of interest, for example by Staudinger ligation or cycloaddition with an alkyne, is not disclosed. The transfer of a galactose moiety comprising a C2-substituted keto group (C2-keto-Gal) to the terminal GlcNAc residues of a G0 glycoform glycan, as well as the linking of C2-keto-Gal to aminooxy biotin, is also disclosed.

Most recently, the click approach based on enzymatic introduction of GalNAz was further extended to a copper-less version for the site-selective radiolabeling of antibodies on the heavy chain glycans.

Efforts from our own laboratory along the same line have resulted in the assembly of antibody conjugates of excellent homogeneity by endoglycosaminidase (Endo S) trimming of the glycan at N297, prior to GalT(Y289L) transfer of GalNAz to the resulting core GlcNAc moiety. Alternatively, mammalian expression of a monoclonal antibody in CHO in the presence of the mannosidase inhibitor swainsonine also generated a mAb featuring a single GlcNAc moiety suitable for GalT transfer (FIG. 4, right) and subsequent conjugation, thereby generation antibody conjugates of similar homogeneity but with a longer glycan spacer between protein and functional group. In particular, the above approaches were applied to conjugate a highly potent toxin to a monoclonal antibody, thereby generating an antibody-drug conjugate with high homogeneity (drug-antibody ratio is 2.0) and stability.

In summary, GalT(Y289L) has been found suitable for transfer of unnatural variants of GalNAc, either by substitution of the amide nitrogen by a methylene group or by appending of the (relatively small) azide functionality. Transfer of other GalNAc variants under the action of a GalT mutant have not been disclosed to date.

### SUMMARY OF THE INVENTION

The present invention relates to a process for attaching an N-acetylgalactosamine-(hetero)aryl moiety to an N-acetylglucosamine moiety, the process comprising the step of contacting the N-acetylgalactosamine-(hetero)aryl moiety with the N-acetylglucosamine moiety in the presence of a mutant galactosyltransferase;
wherein the N-acetylglucosamine moiety is according to Formula (1):

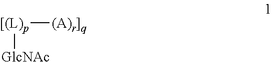

wherein:
p is 0 or 1;
q is 0 or 1;
r is 1, 2, 3 or 4;
with the proviso that when q is 1 and p is 0, then r is 1;
L is a linker;
A is independently selected from the group consisting of D, E or Q, wherein D, E and Q are as defined below;
D is a molecule of interest, preferably selected from the group consisting of a reporter molecule, a diagnostic compound, an active substance, an enzyme, an amino acid, a (non-catalytic) protein, a peptide, a polypeptide, an oligonucleotide, a monosaccharide, an oligosaccharide, a polysaccharide, a glycan, a (poly)ethylene glycol diamine, a polyethylene glycol chain, a polyethylene oxide chain, a polypropylene glycol chain, a polypropylene oxide chain and a 1,x-diaminoalkane (wherein x is the number of carbon atoms in the alkane);
E is a solid surface, preferably selected from the group consisting of functional surfaces, nanomaterials, carbon nanotubes, fullerenes, virus capsids, metal surfaces, metal alloy surfaces and polymer surfaces; and
Q is a functional group, preferably selected from the group consisting of hydrogen, halogen, R³, —CH═C(R³)₂, —C≡CR³, —[C(R³)₂C(R³)₂O]_{q}—R³ wherein q is in the range of 1 to 200, —CN, —N₃, —NCX, —XCN, —XR³, —N(R³)₂, —⁺N(R³)₃, —C(X)N(R³)₂, —C(R³)₂XR³, —C(X)R³, —C(X)XR³, —S(O)R³, —S(O)₂R³, —S(O)OR³, —S(O)₂OR³, —S(O)N(R³)₂, —S(O)₂N(R³)₂, —OS(O)₂R³, —OS(O)OR³, —OS(O)₂OR³, —P(O)(R³)(OR³), —P(O)(OR³)₂, —OP(O)(OR³)₂, —Si(R³)₃, —XC(X)R³, —XC(X)XR³, —XC(X)N(R³)₂, —N(R³)C(X)R³, —N(R³)C(X)XR³ and —N(R³)C(X)N(R³)₂, wherein X is oxygen or sulphur and wherein R³ is independently selected from the group consisting of hydrogen, halogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups optionally substituted and optionally interrupted by one or more heteroatoms selected from O and N;
and wherein the N-acetylgalactosamine-(hetero)aryl moiety is according to Formula (2):

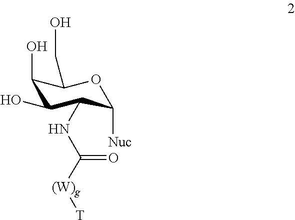

wherein:
g is 0 or 1;
T is a (hetero)aryl group, wherein the (hetero)aryl group is optionally substituted;
Nuc is a nucleotide; and
W is selected from the group consisting of C₁-C₂₄ alkylene groups, C₂-C₂₄ alkenylene groups, C₃-C₂₄ cycloalkylene groups, C₂-C₂₄ (hetero)arylene groups, C₃-C₂₄ alkyl(hetero)arylene groups and C₃-C₂₄ (hetero)arylalkylene groups, wherein the alkylene groups, alkenylene groups, cycloalkylene groups, (hetero)arylene groups, alkyl(hetero)arylene groups and (hetero)arylalkylene groups are optionally substituted, and wherein the alkylene groups, alkenylene groups, cycloalkylene groups, (hetero)arylene groups, alkyl(hetero)arylene groups and (hetero)arylalkylene groups are optionally interrupted by one or more heteroatoms selected from the group consisting of O, S and N.

The invention also relates to a glycoprotein according to Formula (8) or (9):

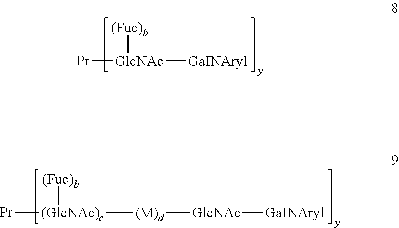

wherein:
y is 1-20;
b is 0 or 1;
c is 0 or 1;
d is 0 or 1;
Pr is a glycoprotein; and
M is a monosaccharide, or a linear or branched oligosaccharide comprising 2 to 20 saccharide moieties; and
wherein GalNAryl is according to Formula (6):

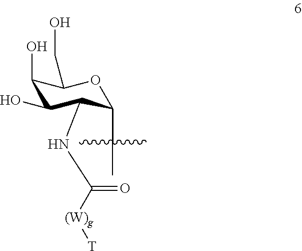

wherein:
W, T and g are as defined above; and
T is optionally substituted.

The invention further relates to a compound according to formula (3b):

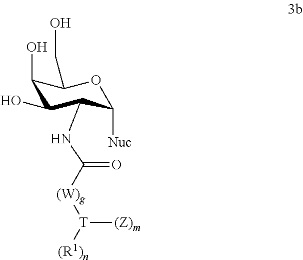

wherein:
Nuc, W, T, Z and R¹ are as defined above;
g is 0;
m is 0, 1, 2, 3, 4, 5, 6, 7 or 8; and
n is0, 1, 2, 3, 4, 5, 6, 7 or 8.

In addition, the invention relates to a compound according to Formula (23b):

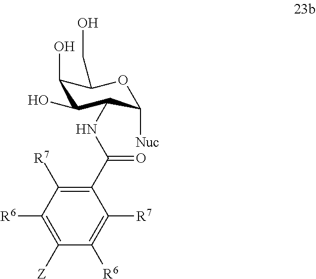

wherein:
Nuc is a nucleotide;
Z is a functional group;
R⁶ is independently selected from the group consisting of hydrogen, F, Cl, Br and I; and
R⁷ is independently selected from the group consisting of hydrogen, F, Cl, Br and I.

### DESCRIPTION OF THE FIGURES

FIG. 1 shows a schematic depiction of the galactosylation of a GlcNAc substrate upon the action of a galactosyltransferase in the presence of UDP-Gal.

In FIG. 2 the structures of different UDP-sugars is represented, modified at C-2′ or C-6′.

FIG. 3 displays the enzymatic transfer of non-natural UDP-sugars onto a GlcNAc derivative. Top figure shows how native GalT is able to transfer, apart from UDP-Gal, also some 6′-modified UDP-galactose derivatives. Bottom figure shows that specific GalT mutants are able to transfer UDP-GalNAc as well as some synthetic variants thereof unto the GlcNAc substrate. The latter may vary from small molecule to glycolipid to glycoprotein.

In FIG. 4, a schematic representation of different glycoproteins is provided, all of which harbor an N-terminal GlcNAc. N-glycoprotein on the right is the result of expression of an N-glycoprotein in CHO in the presence of swainsonine.

In FIG. 5 the synthesis method of UDP-GalNAryl compounds according to Formula (21), (21b), (22), (23), (23b) and (24) is schematically shown.

FIG. 6 shows the schematic scheme for the transfer of furan-modified UDP-GalNAc substrate (22) onto GlcNAc-4-methylumbelliferin upon subjecting to GalT(Y289L).

FIG. 7 shows the schematic scheme for the transfer of either of the modified UDP-GalNAryl substrates (21)-(24) onto the core N-GlcNAc of antibody upon subjecting antibody consecutively to trimming with Endo S, then GalT(Y289L), leading to modified antibodies (28)-(31), respectively.

In FIG. 8, the mass spectrometric analysis is given of trastuzumab heavy chain after consecutive Endo S trimming (top) and subjection to GalT(Y289L) in the presence of UDP-F₂-GalNBAz (23).

In FIG. 9, the mass spectrometric analysis is given of trastuzumab heavy chain after consecutive Endo S trimming (top) and after subjection to GalT(Y289L) in the presence of UDP-GalNfuran (22).

FIG. 10 shows the SDS-PAGE of the heavy chain of trastuzumab derivatives N-azidoacetyl-D-galactosamine (Trast-(GalNAz)₂, top gel) or (Trast-(F₂GalNBAz)₂, lower gel) (as depicted in FIG. 7 obtained by sequential trimming of trastuzumab with Endo S, then GalT(Y289L)-mediated enzymatic transfer from UDP-GalNAz or UDP-GalNBAz (23), respectively), before conjugation to BCN-PEG₂₀₀₀ (lower band in gel) and after conjugation to BCN-PEG₂₀₀₀ (upper band in gel).

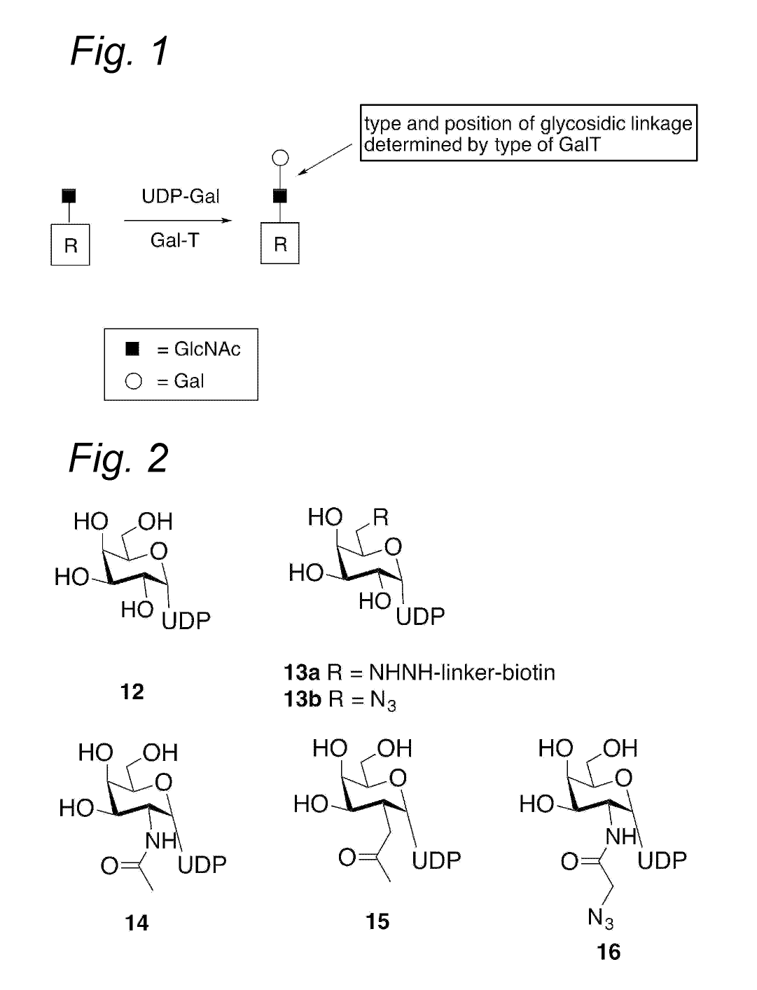

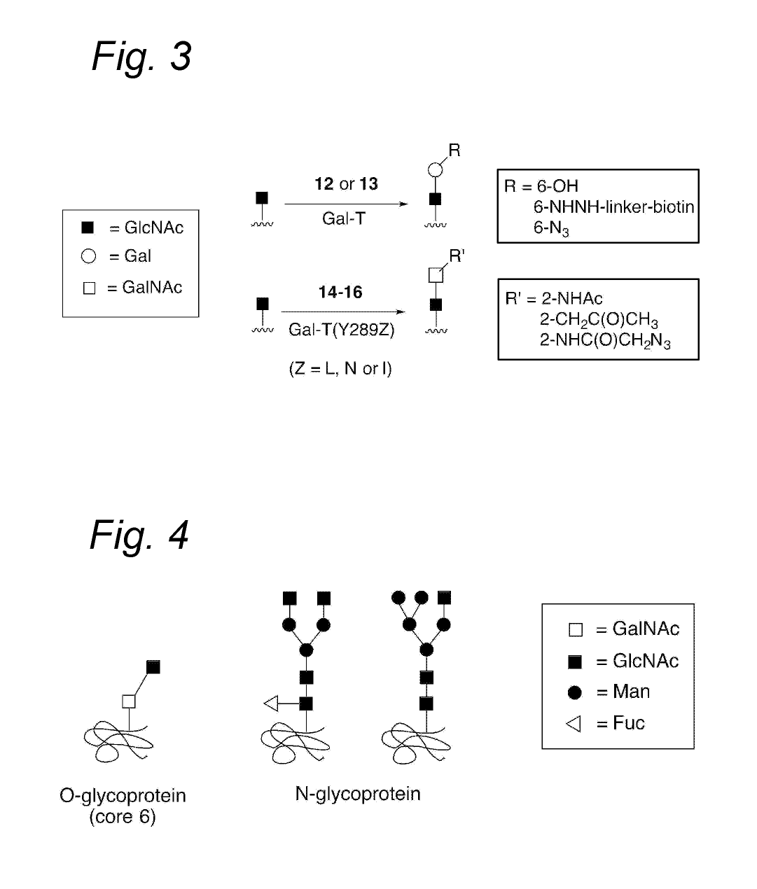

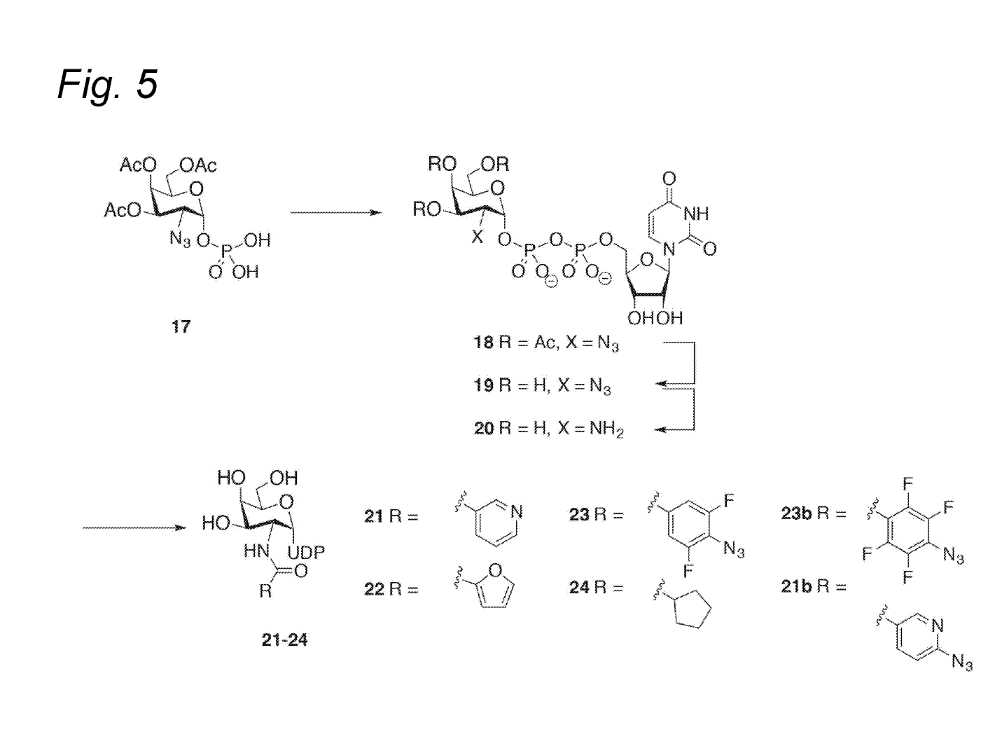

### DETAILED DESCRIPTION OF THE INVENTION

### Definitions

The verb “to comprise” as is used in this description and in the claims and its conjugations is used in its non-limiting sense to mean that items following the word are included, but items not specifically mentioned are not excluded.

In addition, reference to an element by the indefinite article “a” or “an” does not exclude the possibility that more than one of the element is present, unless the context clearly requires that there is one and only one of the elements. The indefinite article “a” or “an” thus usually means “at least one”.

The compounds disclosed in this description and in the claims may comprise one or more asymmetric centres, and different diastereomers and/or enantiomers may exist of the compounds. The description of any compound in this description and in the claims is meant to include both the individual enantiomers, as well as any mixture, racemic or otherwise, of the enantiomers, unless stated otherwise. When the structure of a compound is depicted as a specific enantiomer, it is to be understood that the invention of the present application is not limited to that specific enantiomer.

The compounds may occur in different tautomeric forms. The compounds according to the invention are meant to include all tautomeric forms, unless stated otherwise. When the structure of a compound is depicted as a specific tautomer, it is to be understood that the invention of the present application is not limited to that specific tautomer.

Unsubstituted alkyl groups have the general formula CₙH₂ₙ₊₁ and may be linear or branched. Optionally, the alkyl groups are substituted by one or more substituents further specified in this document. Examples of alkyl groups include methyl, ethyl, propyl, 2-propyl, t-butyl, 1-hexyl, 1-dodecyl, etc.

Unsubstituted cycloalkyl groups comprise at least three carbon atoms and have the general formula CₙH₂ₙ₋₁. Optionally, the cycloalkyl groups are substituted by one or more substituents further specified in this document. Examples of cycloalkyl groups include cyclopropyl, cyclobutyl, cyclopentyl, cyclohexyl, etc.

An aryl group comprises six to twelve carbon atoms and may include monocyclic and bicyclic structures. Optionally, the aryl group may be substituted by one or more substituents further specified in this document. Examples of aryl groups are phenyl and naphthyl.

Arylalkyl groups and alkylaryl groups comprise at least seven carbon atoms and may include monocyclic and bicyclic structures. Optionally, the arylalkyl groups and alkylaryl may be substituted by one or more substituents further specified in this document. An arylalkyl group is for example benzyl. An alkylaryl group is for example 4-t-butylphenyl.

Heteroaryl groups comprise at least two carbon atoms (i.e. at least C₂) and one or 0more heteroatoms N, O, P or S. A heteroaryl group may have a monocyclic or a bicyclic structure. Optionally, the heteroaryl group may be substituted by one or more substituents further specified in this document. Examples of suitable heteroaryl groups include pyridinyl, quinolinyl, pyrimidinyl, pyrazinyl, pyrazolyl, imidazolyl, thiazolyl, pyrrolyl, furanyl, triazolyl, benzofuranyl, indolyl, purinyl, benzoxazolyl, thienyl, phospholyl and oxazolyl.

Heteroarylalkyl groups and alkylheteroaryl groups comprise at least three carbon atoms (i.e. at least C₃) and may include monocyclic and bicyclic structures. Optionally, the heteroaryl groups may be substituted by one or more substituents further specified in this document.

Where an aryl group is denoted as a (hetero)aryl group, the notation is meant to include an aryl group and a heteroaryl group. Similarly, an alkyl(hetero)aryl group is meant to include an alkylaryl group and a alkylheteroaryl group, and (hetero)arylalkyl is meant to include an arylalkyl group and a heteroarylalkyl group. A C₂-C₂₄ (hetero)aryl group is thus to be interpreted as including a C₂-C₂₄ heteroaryl group and a C₆-C₂₄ aryl group. Similary, a C₃-C₂₄ alkyl(hetero)aryl group is meant to include a C₇-C₂₄ alkylaryl group and a C₃-C₂₄ alkylheteroaryl group, and a C₃-C₂₄ (hetero)arylalkyl is meant to include a C₇-C₂₄ arylalkyl group and a C₃-C₂₄ heteroarylalkyl group.

Unless stated otherwise alkyl groups, alkenyl groups, alkenes, alkynes, (hetero)aryl groups, (hetero)arylalkyl groups, alkyl(hetero)aryl groups, alkylene groups, alkenylene groups, cycloalkylene groups, (hetero)arylene groups, alkyl(hetero)arylene groups, (hetero)arylalkylene groups, alkenyl groups, alkynyl groups, cycloalkyl groups, alkoxy groups, alkenyloxy groups, (hetero)aryloxy groups, alkynyloxy groups and cycloalkyloxy groups may be substituted with one or more substituents independently selected from the group consisting of C₁-C₁₂ alkyl groups, C₂-C₁₂ alkenyl groups, C₂-C₁₂ alkynyl groups, C₃-C₁₂ cycloalkyl groups, C₅-C₁₂ cycloalkenyl groups, C₈-C₁₂ cycloalkynyl groups, C₁-C₁₂ alkoxy groups, C₂-C₁₂ alkenyloxy groups, C₂-C₁₂ alkynyloxy groups, C₃-C₁₂ cycloalkyloxy groups, halogens, amino groups, oxo and silyl groups, wherein the silyl groups can be represented by the formula (R²)₃Si—, wherein R² is independently selected from the group consisting of C₁-C₁₂ alkyl groups, C₂-C₁₂ alkenyl groups, C₂-C₁₂ alkynyl groups, C₃-C₁₂ cycloalkyl groups, C₁-C₁₂ alkoxy groups, C₂-C₁₂ alkenyloxy groups, C₂-C₁₂ alkynyloxy groups and C₃-C₁₂ cycloalkyloxy groups, wherein the alkyl groups, alkenyl groups, alkynyl groups, cycloalkyl groups, alkoxy groups, alkenyloxy groups, alkynyloxy groups and cycloalkyloxy groups are optionally substituted, the alkyl groups, the alkoxy groups, the cycloalkyl groups and the cycloalkoxy groups being optionally interrupted by one of more hetero-atoms selected from the group consisting of O, N and S.

An alkynyl group comprises a carbon-carbon triple bond. An unsubstituted alkynyl group comprising one triple bond has the general formula CₙH₂ₙ₋₃. A terminal alkynyl is an alkynyl group wherein the triple bond is located at a terminal position of a carbon chain. Optionally, the alkynyl group is substituted by one more substituents further specified in this document, and/or interrupted by heteroatoms selected from the group of oxygen, nitrogen and sulphur. Examples of alkynyl groups include ethynyl, propynyl, butynyl, octynyl, etc.

A cycloalkynyl group is a cyclic alkynyl group. An unsubstituted cycloalkynyl group comprising one triple bond has the general formula CₙH₂ₙ₋₅. Optionally, a cycloalkynyl group is substituted by one or more substituents further specified in this document. An example of a cycloalkynyl group is cyclooctynyl.

A heterocycloalkynyl group is a cycloalkynyl group interrupted by heteroatoms selected from the group of oxygen, nitrogen and sulphur. Optionally, a heterocycloalkynyl group is substituted by one or more substituents further specified in this document. An example of a heterocycloalkynyl group is azacyclooctynyl.

A (hetero)aryl group comprises an aryl group and a heteroaryl group. An alkyl(hetero)aryl group comprises an alkylaryl group and an alkylheteroaryl group. A (hetero)arylalkyl group comprises a arylalkyl group and a heteroarylalkyl groups. A (hetero)alkynyl group comprises an alkynyl group and a heteroalkynyl group. A (hetero)cycloalkynyl group comprises an cycloalkynyl group and a heterocycloalkynyl group.

A (hetero)cycloalkyne compound is herein defined as a compound comprising a (hetero)cycloalkynyl group.

Several of the compounds disclosed in this description and in the claims may be described as fused (hetero)cycloalkyne compounds, i.e. (hetero)cycloalkyne compounds wherein a second ring structure is fused, i.e. annulated, to the (hetero)cycloalkynyl group. For example in a fused (hetero)cyclooctyne compound, a cycloalkyl (e.g. a cyclopropyl) or an arene (e.g. benzene) may be annulated to the (hetero)cyclooctynyl group. The triple bond of the (hetero)cyclooctynyl group in a fused (hetero)cyclooctyne compound may be located on either one of the three possible locations, i.e. on the 2, 3 or 4 position of the cyclooctyne moiety (numbering according to “IUPAC Nomenclature of Organic Chemistry”, Rule A31.2). The description of any fused (hetero)cyclooctyne compound in this description and in the claims is meant to include all three individual regioisomers of the cyclooctyne moiety.

The general term “sugar” is herein used to indicate a monosaccharide, for example glucose (Glc), galactose (Gal), mannose (Man) and fucose (Fuc). The term “sugar derivative” is herein used to indicate a derivative of a monosaccharide sugar, i.e. a monosaccharide sugar comprising substituents and/or functional groups. Examples of a sugar derivative include amino sugars and sugar acids, e.g. glucosamine (GlcNH₂), galactosamine (GalNH₂) N-acetylglucosamine (GlcNAc), N-acetylgalactosamine (GalNAc), sialic acid (Sia) which is also referred to as N-acetylneuraminic acid (NeuNAc), and N-acetylmuramic acid (MurNAc), glucuronic acid (GlcA) and iduronic acid (IdoA). Examples of a sugar derivative also include compounds herein denoted Su(A)ₓ, wherein Su is a sugar or a sugar derivative, and wherein Su comprises x functional groups A.

The term “nucleotide” is herein used in its normal scientific meaning. The term “nucleotide” refers to a molecule that is composed of a nucleobase, a five-carbon sugar (either ribose or 2-deoxyribose), and one, two or three phosphate groups. Without the phosphate group, the nucleobase and sugar compose a nucleoside. A nucleotide can thus also be called a nucleoside monophosphate, a nucleoside diphosphate or a nucleoside triphosphate. The nucleobase may be adenine, guanine, cytosine, uracil or thymine. Examples of a nucleotide include uridine diphosphate (UDP), guanosine diphosphate (GDP), thymidine diphosphate (TDP), cytidine diphosphate (CDP) and cytidine monophosphate (CMP).

The term “protein” is herein used in its normal scientific meaning. Herein, polypeptides comprising about 10 or more amino acids are considered proteins. A protein may comprise natural, but also unnatural amino acids.

The term “glycoprotein” is herein used in its normal scientific meaning and refers to a protein comprising one or more monosaccharide or oligosaccharide chains (“glycans”) covalently bonded to the protein. A glycan may be attached to a hydroxyl group on the protein (O-linked-glycan), e.g. to the hydroxyl group of serine, threonine, tyrosine, hydroxylysine or hydroxyproline, or to an amide function on the protein (N-glycoprotein), e.g. asparagine or arginine, or to a carbon on the protein (C-glycoprotein), e.g. tryptophan. A glycoprotein may comprise more than one glycan, may comprise a combination of one or more monosaccharide and one or more oligosaccharide glycans, and may comprise a combination of N-linked, O-linked and C-linked glycans. It is estimated that more than 50% of all proteins have some form of glycosylation and therefore qualify as glycoprotein. Examples of glycoproteins include PSMA (prostate-specific membrane antigen), CAL (candida antartica lipase), gp41, gp120, EPO (erythropoietin), antifreeze protein and antibodies.

The term “glycan” is herein used in its normal scientific meaning and refers to a monosaccharide or oligosaccharide chain that is linked to a protein. The term glycan thus refers to the carbohydrate-part of a glycoprotein. The glycan is attached to a protein via the C-1 carbon of one sugar, which may be without further substitution (monosaccharide) or may be further substituted at one or more of its hydroxyl groups (oligosaccharide). A naturally occurring glycan typically comprises 1 to about 10 saccharide moieties. However, when a longer saccharide chain is linked to a protein, said saccharide chain is herein also considered a glycan.

A glycan of a glycoprotein may be a monosaccharide. Typically, a monosaccharide glycan of a glycoprotein consists of a single N-acetylglucosamine (GlcNAc), glucose (Glc), mannose (Man) or fucose (Fuc) covalently attached to the protein.

A glycan may also be an oligosaccharide. An oligosaccharide chain of a glycoprotein may be linear or branched. In an oligosaccharide, the sugar that is directly attached to the protein is called the core sugar. In an oligosaccharide, a sugar that is not directly attached to the protein and is attached to at least two other sugars is called an internal sugar. In an oligosaccharide, a sugar that is not directly attached to the protein but to a single other sugar, i.e. carrying no further sugar substitutents at one or more of its other hydroxyl groups, is called the terminal sugar. For the avoidance of doubt, there may exist multiple terminal sugars in an oligosaccharide of a glycoprotein, but only one core sugar.

A glycan may be an O-linked glycan, an N-linked glycan or a C-linked glycan. In an O-linked glycan a monosaccharide or oligosaccharide glycan is bonded to an O-atom in an amino acid of the protein, typically via a hydroxyl group of serine (Ser) or threonine (Thr). In an N-linked glycan a monosaccharide or oligosaccharide glycan is bonded to the protein via an N-atom in an amino acid of the protein, typically via an amide nitrogen in the side chain of asparagine (Asn) or arginine (Arg). In a C-linked glycan a monosaccharide or oligosaccharide glycan is bonded to a C-atom in an amino acid of the protein, typically to a C-atom of tryptophan (Trp).

The end of an oligosaccharide that is directly attached to the protein is called the reducing end of a glycan. The other end of the oligosaccharide is called the non-reducing end of a glycan.

For O-linked glycans, a wide diversity of chains exist. Naturally occurring O-linked glycans typically feature a serine or threonine-linked α-O-GalNAc moiety, further substituted with galactose, sialic acid and/or fucose. The hydroxylated amino acid that carries the glycan substitution may be part of any amino acid sequence in the protein.

For N-linked glycans, a wide diversity of chains exist. Naturally occurring N-linked glycans typically feature an asparagine-linked β-N-GlcNAc moiety, in turn further substituted at its 4—OH with β-GlcNAc, in turn further substituted at its 4—OH with β-Man, in turn further substituted at its 3—OH and 6—OH with α-Man, leading to the glycan pentasaccharide Man₃GlcNAc₂. The core GlcNAc moiety may be further substituted at its 6—OH by α-Fuc. The pentasaccharide Man₃GlcNAc₂ is the common oligosaccharide scaffold of nearly all N-linked glycoproteins and may carry a wide variety of other substituents, including but not limited to Man, GlcNAc, Gal and sialic acid. The asparagine that is substituted with the glycan on its side-chain is typically part of the sequence Asn-X-Ser/Thr, with X being any amino acid but proline and Ser/Thr being either serine or threonine.

The term “antibody” is herein used in its normal scientific meaning. An antibody is a protein generated by the immune system that is capable of recognizing and binding to a specific antigen. An antibody is an example of a glycoprotein. The term antibody herein is used in its broadest sense and specifically includes monoclonal antibodies, polyclonal antibodies, dimers, multimers, multispecific antibodies (e.g. bispecific antibodies), antibody fragments, and double and single chain antibodies. The term “antibody” is herein also meant to include human antibodies, humanized antibodies, chimeric antibodies and antibodies specifically binding cancer antigen. The term “antibody” is meant to include whole antibodies, but also fragments of an antibody, for example an antibody Fab fragment, F(ab′)₂, Fv fragment or Fc fragment from a cleaved antibody, a scFv-Fc fragment, a minibody, a diabody or a scFv. Furthermore, the term includes genetically engineered antibodies and derivatives of an antibody. Antibodies, fragments of antibodies and genetically engineered antibodies may be obtained by methods that are known in the art. Suitable marketed antibodies include, amongst others, abciximab, rituximab, basiliximab, palivizumab, infliximab, trastuzumab, alemtuzumab, adalimumab, tositumomab-I131, cetuximab, ibrituximab tiuxetan, omalizumab, bevacizumab, natalizumab, ranibizumab, panitumumab, eculizumab, certolizumab pegol, golimumab, canakinumab, catumaxomab, ustekinumab, tocilizumab, ofatumumab, denosumab, belimumab, ipilimumab and brentuximab.

### Process for Attaching an N-acetylgalactosamine-(hetero)aryl Moiety and an N-acetylglucosamine Moiety

The invention relates to a process for the enzymatic attaching of an N-acetylgalactosamine moiety comprising a (hetero)aryl group to an N-acetylglucosamine moiety, in the presence of a mutant galactosyltransferase.

The present invention relates to a process for attaching an N-acetylgalactosamine-(hetero)aryl moiety to an N-acetylglucosamine moiety, the process comprising the step of contacting the N-acetylgalactosamine-(hetero)aryl moiety with the N-acetylglucosamine moiety in the presence of a mutant galactosyltransferase; wherein the N-acetylglucosamine moiety is according to Formula (1):

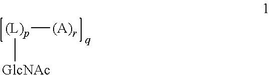

wherein:
p is 0 or 1;
q is 0 or 1;
r is 1, 2, 3 or 4;
with the proviso that when q is 1 and p is 0, then r is 1;
L is a linker;
A is independently selected from the group consisting of D, E or Q, wherein D, E and Q are as defined below;
D is a molecule of interest;
E is a solid surface; and
Q is a functional group;
   and wherein the N-acetylgalactosamine-(hetero)aryl moiety is according to Formula (2):

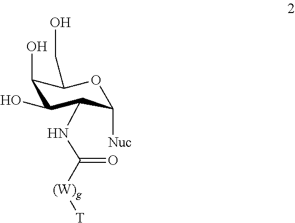

wherein:
g is 0 or 1;
T is a (hetero)aryl group, wherein the (hetero)aryl group is optionally substituted;
Nuc is a nucleotide; and
W is selected from the group consisting of C₁-C₂₄ alkylene groups, C₂-C₂₄ alkenylene groups, C₃-C₂₄ cycloalkylene groups, C₂-C₂₄ (hetero)arylene groups, C₃-C₂₄ alkyl(hetero)arylene groups and C₃-C₂₄ (hetero)arylalkylene groups, wherein the alkylene groups, alkenylene groups, cycloalkylene groups, (hetero)arylene groups, alkyl(hetero)arylene groups and (hetero)arylalkylene groups are optionally substituted, and wherein the alkylene groups, alkenylene groups, cycloalkylene groups, (hetero)arylene groups, alkyl(hetero)arylene groups and (hetero)arylalkylene groups are optionally interrupted by one or more heteroatoms selected from the group consisting of O, S and N.

The N-acetylglucosamine moiety (1) and preferred embodiments thereof, and the N-acetylgalactosamine-(hetero)aryl moiety (2) and preferred embodiments thereof, are described in more detail below.

N-acetylglucosamine is herein also referred to as GlcNAc, and N-acetylgalactosamine is herein also referred to as GalNAc. The terms GlcNAc and GalNAc are well known in the art, and are herein used in their normal scientific meaning.

The N-acetylglucosamine moiety according to Formula (1) is herein also referred to as (A-L)-GlcNAc. The N-acetylgalactosamine-(hetero)aryl moiety according to Formula (2) is herein also referred to as Nuc-GalNAryl. GalNAryl is herein defined as an N-acetylgalactosamine moiety comprising an aryl group or a heteroaryl group. The aryl group or heteroaryl group of GalNAryl is optionally substituted.

Said N-acectylgalactosamine moiety comprising an aryl group or a heteroaryl group, herein also referred to as GalNAryl, is according to Formula (6):

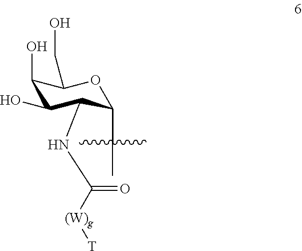

wherein:
W, T and g are as defined above; and
T is optionally substituted.

When GalNAryl (6) is bonded at C1 to e.g. a nucleotide, as described above for (2), said GalNAryl is also referred to as Nuc-GalNAryl. When GalNAryl (6) is bonded at C1 to e.g. a GlcNAc moiety, as described below for (5), said GalNAryl is also referred to as GlcNAc-GalNAryl.

In the process according to the invention, GalNAryl of Nuc-GalNAryl (2) is connected to GlcNAc of (A-L)-GlcNAc (1), in order to obtain a compound according to Formula (5):

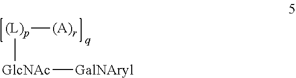

wherein:
L, A, p, r and q are as defined above; and
GalNAryl is according to Formula (6) as defined above.

In other words, the present invention relates to a process for attaching GalNAryl of an N-acetylgalactosamine-(hetero)aryl moiety to GlcNAc of an N-acetylglucosamine moiety, the process comprising the step of contacting the N-acetylgalactosamine-(hetero)aryl moiety with the N-acetylglucosamine moiety in the presence of a mutant galactosyltransferase, wherein the N-acetylglucosamine moiety is according to Formula (1) and wherein the N-acetylgalactosamine-(hetero)aryl moiety is according to Formula (2), in order to obtain a product according to Formula (5), wherein the compounds according to Formula (1), (2) and (5) are as defined above.

In the process according to the invention, GalNAryl of Nuc-GalNAryl is bonded via C1 to GlcNAc of (A-L)-GlcNAc via an O-glycosidic bond. The type of O-glycosidic bond that is formed between the GalNAryl of Nuc-GalNAryl and the GlcNAc of (A-L)-GlcNAc depends on the type of mutant galactosyltransferase that is used in the process according to the invention. The GalNAryl of Nuc-GalNAryl may for example be bonded via C1 to C4 of the GlcNAc via a β(1,4)-glycosidic bond, or to C3 of said GlcNAc via an α(1,3)-glycosidic bond. When the process is performed in the presence of a mutant β(1,4)-galactosyltransferase then binding occurs via C1 of GalNAryl and C4 of GlcNAc via a β(1,4)-glycosidic bond. When the process is performed in the presence of a mutant α(1,3)-galactosyltransferase then binding occurs via C1 of GalNAryl and C3 of GlcNAc via an α(1,3)-glycosidic bond. C1 of GalNAryl refers to C1 of the galactose moiety in GalNAryl, i.e. to the C-atom that nucleotide Nuc is bonded to in Nuc-GalNAryl (2) as defined above.

### Mutant Galactosyltransferase

The process according to the invention is performed in the presence of a mutant galactosyltransferase. Galactosyltransferases and mutant galactosyltransferases are well known in the art.

A mutant galactosyltransferase is herein defined as a galactosyltransferase having an amino acid sequence that is different from the sequence of its counterpart wild-type galactosyltransferase. The mutation may e.g. comprise a single amino acid change (a point mutation), but also a multiple amino acid change (e.g. of 2 to 10, preferably of 2 to 6, more preferably of 2, 3 or 4, even more preferably of 2 amino acids), or a deletion or insertion of one or more (e.g. of 1 to 10, preferably of 1 to 6, more preferably of 1, 2, 3 or 4, even more preferably of 1 or 2) amino acids.

The term “catalytic domain” herein refers to an amino acid segment that folds into a domain that is able to catalyze the linkage of the specific GalNAryl in Nuc-GalNAryl to the GlcNAc in (A-L)-GlcNAc in a specific process according to the invention. The term “mutant catalytic domain” refers to a catalytic domain having an amino acid sequence that is different from the sequence of the catalytic domain of its wild-type counterpart. The mutation may e.g. comprise a single amino acid change (a point mutation), but also a multiple amino acid change (e.g. 2 to 10, preferably 2 to 6, more preferably 2, 3 or 4, even more preferably 2 amino acids), or a deletion or insertion one or more (e.g. 1 to 10, preferably 1 to 6, more preferably 1, 2, 3 or 4, even more preferably 1 or 2) amino acids. Preferably, the mutation comprises a single amino acid change or a multiple amino acid change, i.e. preferably the mutation comprises 1 to 10, preferably 1 to 6, more preferably 1, 2, 3 or 4, even more preferably 1 or 2 amino acid changes. The mutant catalytic domain may be included within a full length galactosyltransferase, but also in recombinant molecules containing the mutant catalytic domain, e.g. a polypeptide fragment or a recombinant polypeptide, optionally linked to additional amino acids.

The term “mutant galactosyltransferase” herein refers to a full-length galactosyltransferase or a fragment thereof, having an amino acid sequence that is different from its counterpart wild-type, but also to recombinant molecules comprising the mutant catalytic domain.

Mutant GalT catalytic domains are for example disclosed in WO 2004/063344 (National Institutes of Health), incorporated by reference herein. WO 2004/063344 discloses Tyr-289 mutants of GalT, which are referred to as Y289L, Y289N and Y289I.

Mutant GalT domains that catalyze the formation of an N-acetylgalactosamine-β(1,4)-N-acetylglucosamine bond are disclosed in WO 2004/063344 (incorporated by reference herein). As was described above, the disclosed mutant GalT domains may be included within full-length GalT enzymes, or in recombinant molecules containing the catalytic domains, as is e.g. disclosed in WO 2004/063344, incorporated by reference herein.

Another mutant GalT domain is for example Y284L, disclosed by Bojarova et al., Glycobiology 2009, 19, 509, incorporated by reference herein. The mutation in position 284 concerns a tyrosine residue.

Another mutant GalT domain is for example R228K, disclosed by Qasba et al., Glycobiology 2002, 12, 691, incorporated by reference herein, wherein Arg228 is replaced by lysine.

In a preferred embodiment of the process according to the invention, the mutant galactosyltransferase is selected from the group consisting of mutant β(1,4)-galactosyltransferases and mutant β(1,3)-N-galactosyltransferases.

In a further preferred embodiment, the mutant β(1,4)-galactosyltransferase is a mutant β(1,4)-galactosyltransferase I. β(1,4)-Galactosyltransferase I is herein also referred to as β(1,4)-GalT or GalT. Even more preferably, the mutant β(1,4)-galactosyltransferase is a mutant bovine or human β(1,4)-galactosyltransferase I.

In a further preferred embodiment, the mutant galactosyltransferase is preferably selected from the group consisting of bovine or human β(1,4)-Gal-T1 mutants GalT Y289L, GalT Y289N, GalT Y289I, Y284L and R228K, more preferably from the group consisting of GalT Y289L, GalT Y289N and GalT Y289I. GalT Y289L, GalT Y289N and GalT Y289I are described in more detail in WO 2004/063344, in Qasba et al., Prot. Expr. Pur. 2003, 30, 219 and in Qasba et al., J. Biol. Chem. 2002, 277, 20833 (all incorporated by reference).

In another preferred embodiment, the mutant galactosyltransferase is a bovine or human β(1,4)-galactosyltransferase T1 mutant. In a further preferred embodiment the bovine or human β(1,4)-galactosyltransferase T1 mutant is selected from the group consisting of GalT Y289F, GalT Y289M, GalT Y289V, GalT Y289G, GalT Y289I and GalT Y289A, more preferably from the group consisting of GalT Y289F and GalT Y289M.

GalT Y289F, GalT Y289M, GalT Y289V, GalT Y289G, GalT Y289I and GalT Y289A may be provided via site-directed mutagenesis processes, in a similar manner as disclosed in WO 2004/063344, in Qasba et al., Prot. Expr. Pur. 2003, 30, 219 and in Qasba et al., J. Biol. Chem. 2002, 277, 20833 (all incorporated by reference) for Y289L, Y289N and Y289I. In GalT Y289L the tyrosine amino acid (Y) at position 289 is replaced by a leucine (L) amino acid, in GalT Y289N said tyrosine is replaced by an asparagine (N) amino acid, and in Y289I said tyrosine is replaced by an isoleucine (I) amino acid. In GalT Y289F the tyrosine amino acid (Y) at position 289 is replaced by a phenyl alanine (F) amino acid, in GalT Y289M said tyrosine is replaced by a methionine (M) amino acid, in GalT Y289V by a valine (V) amino acid, in GalT Y289G by a glycine (G) amino acid, in GalT Y289I by an isoleucine (I) amino acid and in Y289A by an alanine (A) amino acid.

In a preferred embodiment of the process according to the invention, the mutant galactosyltransferase is selected from the group consisting of mutant bovine or human β(1,4)-Gal-T1 GalT Y289L, GalT Y289N, GalT Y289F, GalT Y289M, GalT Y289V, GalT Y289G, GalT Y289I and GalT Y289A.

In another embodiment of the process according to the invention, the mutant galactosyltransferase is a mutant α(1,3)-N-galactosyltransferase, also referred to as a3Gal-T. Preferably, the α(1,3)-N-galactosyltransferase is an α(1,3)-N-acetylgalactosaminyltransferase, also referred to as a3GalNAc-T, as disclosed in WO 2009/025646, incorporated by reference herein. Mutation of a3Gal-T can broaden donor specificity of the enzyme, and make it an a3GalNAc-T. Polypeptide fragments and catalytic domains of α(1,3)-N-acetylgalactosaminyltransferases are disclosed in WO 2009/025646, incorporated by reference herein.

Preferably, the mutant galactosyltransferase comprises a single amino acid change (a point mutation), or a multiple amino acid change (e.g. of 2 to 10, preferably of 2 to 6, more preferably of 2, 3 or 4, even more preferably of 2 or 3, and yet even more preferably of 2 amino acids).

As described above, when the mutant galactosyltransferase is a bovine or human β(1,4)-galactosyltransferase T1 mutant, it is preferred that the tyrosine amino acid (Y) at position 289 is replaced by a phenyl alanine (F), a methionine (M) amino acid, a valine (V) amino acid, a glycine (G) amino acid, an alanine (A) amino acid, a leucine (L) amino acid, an asparagine (N) amino acid, or an isoleucine (I) amino acid.

In another preferred embodiment, when the mutant galactosyltransferase is a bovine or human β(1,4)-galactosyltransferase T1 mutant, said mutant galactosyltransferase comprises a multiple amino acid change (e.g. of 2 to 10, preferably of 2 to 6, more preferably of 2, 3 or 4, and even more preferably of 2 amino acids). In this embodiment it is further preferred that the tyrosine amino acid at position 289 is replaced (preferably by a phenyl alanine (F), a methionine (M) amino acid, a valine (V) amino acid, a glycine (G) amino acid, an alanine (A) amino acid, a leucine (L) amino acid, an asparagine (N) amino acid or an isoleucine (I) amino acid), and that one or more other amino acids are changed. The one or more additional amino acid changes comprise preferably at least replacement of the cysteine (C) amino acid at position 342, preferably by a threonine (T) amino acid. In other words, in this embodiment it is preferred that the tyrosine amino acid at position 289 is replaced (preferably by a phenyl alanine (F), a methionine (M) amino acid, a valine (V) amino acid, a glycine (G) amino acid, an alanine (A) amino acid, a leucine (L) amino acid, an asparagine (N) amino acid or an isoleucine (I) amino acid) and that the cysteine (C) amino acid at position 342 is replaced, preferably by a threonine (T) amino acid.

In a particularly preferred embodiment, when the mutant galactosyltransferase is a bovine or human β(1,4)-galactosyltransferase T1 mutant, the cysteine (C) amino acid at position 342 is replaced by a threonine (T) amino acid, and the tyrosine (Y) amino acid at position 289 is replaced by a phenyl alanine (F), a methionine (M) amino acid, a valine (V) amino acid, a glycine (G) amino acid, an alanine (A) amino acid, a leucine (L) amino acid, an asparagine (N) amino acid or an isoleucine (I) amino acid).

Therefore, in a particularly preferred embodiment of the process according to the invention, the mutant galactosyltransferase is selected from the group consisting of mutant bovine or human β(1,4)-Gal-T1 GalT Y289L C342T, GalT Y289N C342T, Y289F C342T, GalT Y289M C342T, GalT Y289V C342T, GalT Y289G C342T, GalT Y289I C342T and GalT Y289A C342T.

These mutant galactosyltransferases comprising two amino acid changes may be provided via site-directed mutagenesis processes, in a similar manner as disclosed in WO 2004/063344, in Qasba et al., Prot. Expr. Pur. 2003, 30, 219 and in Qasba et al., J. Biol. Chem. 2002, 277, 20833 (all incorporated by reference).

In a further preferred embodiment, the mutant galactosyltransferase is selected from the group consisting of mutant bovine or human β(1,4)-Gal-T1 GalT Y289L C342T, GalT Y289N C342T, GalT Y289I C342T, GalT Y289M C342T and GalT Y289F C342M. In a further preferred embodiment, the mutant galactosyltransferase is selected from the group consisting of mutant bovine or human β(1,4)-Gal-T1 GalT Y289L C342T, GalT Y289N C342T and GalT Y289I C342T.

In another further preferred embodiment, the mutant galactosyltransferase is selected from the group consisting of mutant bovine or human β(1,4)-Gal-T1 GalT Y289F C342T, GalT Y289M C342T, GalT Y289V C342T, GalT Y289G C342T, GalT Y289I C342T and GalT Y289A C342T, more preferably from the group consisting of Y289M C342T and GalT Y289F C342T.

Preferably, the galactosyltransferase used in a process of the invention is a mutant as defined herein of bovine GalT as defined by SEQ ID NO: 17.

Further preferred is a galactosyltransferase that is a fragment of the full length bovine or human galactosyltransferase or mutant thereof as defined herein, more preferably a fragment of bovine GalT as defined by SEQ ID NO: 17.

In a preferred embodiment, said fragment is a polypeptide consisting of a constitutive amino acid sequence of bovine or human galactosyltransferase as defined herein, preferably bovine galactosyltransferase as defined herein, delimited by the amino acids on position 130 and 402 which is indicated herein as GalT 130-402. Preferably, said fragment is a polypeptide consisting of a constitutive amino acid sequence of any one of SEQ ID NO: 17-24, i.e. any one of SEQ ID NO: 17, SEQ ID NO: 18, SEQ ID NO: 19, SEQ ID NO: 20, SEQ ID NO: 21, SEQ ID NO: 22, SEQ ID NO: 23 and SEQ ID NO: 24, delimited by the amino acids on position 130 and 402 of each of said sequence. Preferably, said fragment has an amino acid sequence of SEQ ID NO: 20 or SEQ ID NO: 25. Preferably, the fragment of the present embodiment is expressed using Escherichia coli (E. coli) as a host cell.

In a preferred embodiment, said fragment is a polypeptide consisting of a constitutive amino acid sequence of bovine or human galactosyltransferase as defined herein, preferably bovine galactosyltransferase as defined herein, delimited by the amino acids on position 74 and 402, indicated herein as GalT 74-402. Preferably, said fragment is a polypeptide consisting of a constitutive amino acid sequence of any one of SEQ ID NO: 17-24, i.e. any one of SEQ ID NO: 17, SEQ ID NO: 18, SEQ ID NO: 19, SEQ ID NO: 20, SEQ ID NO: 21, SEQ ID NO: 22, SEQ ID NO: 23 and SEQ ID NO: 24, delimited by the amino acids on position 74 and 402 of each of said sequence. Preferably, said fragment has an amino acid sequence of any one of SEQ ID NO: 26-33, i.e. any one of SEQ ID NO: 26, SEQ ID NO: 27, SEQ ID NO: 28, SEQ ID NO: 29, SEQ ID NO: 30, SEQ ID NO: 31, SEQ ID NO: 32 and SEQ ID NO: 33. Most preferably, said fragment has an amino acid sequence of SEQ ID NO: 32 or SEQ ID NO: 33. Preferably, the fragment of the present embodiment is expressed using CHO as a host cell.

The process according to the invention is preferably performed in a suitable buffer solution, such as for example phosphate, buffered saline (e.g. phosphate-buffered saline, tris-buffered saline), citrate, HEPES, tris and glycine. Suitable buffers are known in the art. Preferably, the buffer solution is phosphate-buffered saline (PBS) or tris buffer.

The process is preferably performed at a temperature in the range of about 4 to about 50° C., more preferably in the range of about 10 to about 45° C., even more preferably in the range of about 20 to about 40° C., and most preferably in the range of about 30 to about 37° C.

The process is preferably performed a pH in the range of about 5 to about 9, preferably in the range of about 5.5 to about 8.5, more preferably in the range of about 6 to about 8. Most preferably, the process is performed at a pH in the range of about 7 to about 8.

### N-acetylgalactosamine-(hetero)aryl Moiety

As described above, in the process according to the invention, the N-acetylgalactosamine-(hetero)aryl moiety, also referred to as Nuc-GalNAryl, is according to Formula (2):

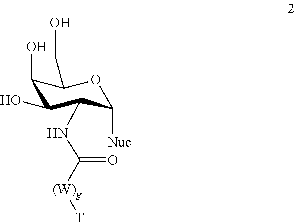

wherein W, T (optionally substituted), Nuc and g are as defined above.

As was described above, the N-acetylgalactosamine-(hetero)aryl moiety according to Formula (2) is herein also referred to as Nuc-GalNAryl. The term GalNAryl herein refers to a moiety according to Formula (6):

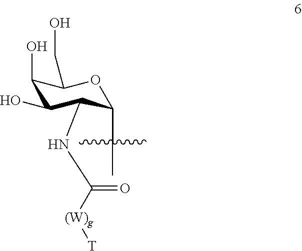

wherein W, T (optionally substituted) and g are as defined above.

The term “Nuc” herein refers to a nucleotide. Nucleotides are well known in the art, and the term “nucleotide” is herein used in its normal scientific meaning. In the process according to the invention, Nuc is preferably selected from the group consisting of a nucleoside monophosphate and a nucleoside diphosphate, more preferably from the group consisting of uridine diphosphate (UDP), guanosine diphosphate (GDP), thymidine diphosphate (TDP), cytidine diphosphate (CDP) and cytidine monophosphate (CMP), more preferably from the group consisting of uridine diphosphate (UDP), guanosine diphosphate (GDP), cytidine diphosphate and (CDP). Most preferably, Nuc is UDP.

Throughout this description, the claims and the drawings, when the nucleotide is UDP, i.e. when -Nuc is -UDP, the nucleotide has the structure shown below.

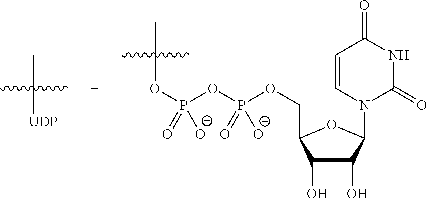

When the nucleotide Nuc is e.g. UDP, then the corresponding Nuc-GalNAryl (2) as defined above is referred to as UDP-GalNAryl. In analogy, when Nuc is e.g. CDP, then the corresponding Nuc-GalNAryl (2) as defined above is also referred to as CDP-GalNAryl. In the process according to the invention, Nuc-GalNAryl is thus preferably selected from the group consisting of UDP-GalNAryl, GDP-GalNAryl, TDP-GalNAryl, CDP-GalNAryl and CMP-GalNAryl, more preferably from the group consisting of UDP-GalNAryl, GDP-GalNAryl and CDP-GalNAryl. Most preferably, Nuc-GalNAryl is UDP-GalNAryl.

Moiety W in (5) is optionally present (g is 0 or 1), and consequently (hetero)aryl group T is either bonded directly to the to the C-atom of the C(O) group (g is 0), or connected to said C-atom via moiety W (g is 1). In a preferred embodiment, g is 0, i.e. W is absent. In another preferred embodiment, g is 1.

W is selected from the group consisting of C₁-C₂₄ alkylene groups, C₂-C₂₄ alkenylene groups, C₃-C₂₄ cycloalkylene groups, C₂-C₂₄ (hetero)arylene groups, C₃-C₂₄ alkyl(hetero)arylene groups and C₃-C₂₄ (hetero)arylalkylene groups, wherein the alkylene groups, alkenylene groups, cycloalkylene groups, (hetero)arylene groups, alkyl(hetero)arylene groups and (hetero)arylalkylene groups are optionally substituted, and wherein the alkylene groups, alkenylene groups, cycloalkylene groups, (hetero)arylene groups, alkyl(hetero)arylene groups and (hetero)arylalkylene groups are optionally interrupted by one or more heteroatoms selected from the group consisting of O, S and N.

Preferably, W is selected from the group consisting of C₁-C₁₂ alkylene groups, C₂-C₁₂ alkenylene groups, C₃-C₁₂ cycloalkylene groups, C₂-C₁₂ (hetero)arylene groups, C₃-C₁₂ alkyl(hetero)arylene groups and C₃-C₁₂ (hetero)arylalkylene groups, wherein the alkylene groups, alkenylene groups, cycloalkylene groups, (hetero)arylene groups, alkyl(hetero)arylene groups and (hetero)arylalkylene groups are optionally substituted, and wherein the alkylene groups, alkenylene groups, cycloalkylene groups, (hetero)arylene groups, alkyl(hetero)arylene groups and (hetero)arylalkylene groups are optionally interrupted by one or more heteroatoms selected from the group consisting of O, S and N.

More preferably, W is selected from the group consisting of C₁-C₆ alkylene groups, C₂-C₆ alkenylene groups, C₃-C₆ cycloalkylene groups, C₂-C₈ (hetero)arylene groups, C₃-C₆ alkyl(hetero)arylene groups and C₃-C₆ (hetero)arylalkylene groups, wherein the alkylene groups, alkenylene groups, cycloalkylene groups, (hetero)arylene groups, alkyl(hetero)arylene groups and (hetero)arylalkylene groups are optionally substituted, and wherein the alkylene groups, alkenylene groups, cycloalkylene groups, (hetero)arylene groups, alkyl (hetero)aryl ene groups and (hetero)arylalkylene groups are optionally interrupted by one or more heteroatoms selected from the group consisting of O, S and N.

More preferably, W is selected from the group consisting of C₁-C₆ alkylene groups and C₂-C₈ (hetero)arylene groups, preferably C₁-C₆ alkylene groups.

Even more preferably, W is selected from the group consisting of methylene, ethylene, propylene, butylene (preferably n-butylene), pentylene (preferably n-pentylene) and hexylene (preferably n-hexylene). Yet even more preferably W is methylene, ethylene or propylene, preferably methylene or ethylene and most preferably W is methylene.

T is a (hetero)aryl group, wherein the (hetero)aryl group is optionally substituted. The term “(hetero)aryl group” herein refers to aryl groups as well as to heteroaryl groups. The term “(hetero)aryl group” herein refers to monocyclic (hetero)aryl groups as well as to bicyclic (hetero)aryl groups. The (hetero)aryl group in the N-acetylgalactosamine-(hetero)aryl moiety according to Formula (2) may be any aryl group or any heteroaryl group.

In a preferred embodiment of the process according to the invention, the (hetero)aryl group in Nuc-GalNAryl according to Formula (2) is selected from the group consisting of phenyl groups, naphthyl groups, anthracyl groups, pyrrolyl groups, pyrrolium groups, furanyl groups, thiophenyl groups (i.e. thiofuranyl groups), pyrazolyl groups, imidazolyl groups, isoxazolyl groups, oxazolyl groups, oxazolium groups, isothiazolyl groups, thiazolyl groups, 1,2,3-triazolyl groups, 1,3,4-triazolyl groups, diazolyl groups, 1-oxa-2,3-diazolyl groups, 1-oxa-2,4-diazolyl groups, 1-oxa-2,5-diazolyl groups, 1-oxa-3,4-diazolyl groups, 1-thia-2,3-diazolyl groups, 1-thia-2,4-diazolyl groups, 1-thia-2,5-diazolyl groups, 1-thia-3,4-diazolyl groups, tetrazolyl groups pyridinyl groups, pyridazinyl groups, pyrimidinyl groups, pyrazinyl groups, pyradizinyl groups, pyridiniumyl groups, pyrimidinium groups, benzofuranyl groups, benzothiophenyl groups, benzimidazolyl groups, indazolyl groups, benzotriazolyl groups, pyrrolo[2,3-b]pyridinyl groups, pyrrolo[2,3-c]pyridinyl groups, pyrrolo[3,2-c]pyridinyl groups, pyrrolo[3,2-b]pyridinyl groups, imidazo[4,5-b]pyridinyl groups, imidazo[4,5-c]pyridinyl groups, pyrazolo[4,3-d]pyridinyl groups, pyrazolo[4,3-c]pyridinyl groups, pyrazolo[3,4-c]pyridinyl groups, pyrazolo[3,4-b]pyridinyl groups, isoindolyl groups, indazolyl groups, purinyl groups, indolininyl groups, imidazo[1,2-a]pyridinyl groups, imidazo[1,5-a]pyridinyl groups, pyrazolo[1,5-a]pyridinyl groups, pyrrolo[1,2-b]pyridazinyl groups, imidazo[1,2-c]pyrimidinyl groups, quinolinyl groups, isoquinolinyl groups, cinnolinyl groups, quinazolinyl groups, quinoxalinyl groups, phthalazinyl groups, 1,6-naphthyridinyl groups, 1,7-naphthyridinyl groups, 1,8-naphthyridinyl groups, 1,5-naphthyridinyl groups, 2,6-naphthyridinyl groups, 2,7-naphthyridinyl groups, pyrido[3,2-d]pyrimidinyl groups, pyrido[4,3-d]pyridmidinyl groups, pyrdio[3,4-d]pyrimidinyl groups, pyrido[2,3-d]pyrimidinyl groups, pyrido[2,3-b]pyrazinyl groups, pyrido[3,4-b]pyrazinyl groups, pyrimido[5,4-pyrimidinyl groups, pyrazino[2,3-b]pyrazinyl groups and pyrimido[4,5-d]pyrimidinyl groups.

In a further preferred embodiment, the (hetero)aryl group is selected from the group consisting of phenyl groups, pyridinyl groups, pyridiniumyl groups, pyrimidinyl groups, pyrimidinium groups, pyrazinyl groups, pyradizinyl groups, pyrrolyl groups, pyrrolium groups, furanyl groups, thiophenyl groups (i.e. thiofuranyl groups), diazolyl groups, quinolinyl groups, imidazolyl groups, oxazolyl groups and oxazolium groups.

As described above, the aryl group or the heteroaryl group in Nuc-GalNAryl according to Formula (2) is optionally substituted.

In a preferred embodiment, the (hetero)aryl group in Nuc-GalNAryl is unsubstituted.

In another preferred embodiment, the (hetero)aryl group in Nuc-GalNAryl comprises one or more substituents. The (hetero)aryl group may be substituted with any substituent. Suitable substituents include for example all kinds of functional groups, all kinds of hydrocarbon groups (e.g. alkyl, aryl), alkoxy groups, aryloxy groups, alkylamino groups and arylamino groups.

Functional groups are well known in the art. When the (hetero)aryl group is substituted with a functional group, the functional group may for example be a 1,3-dipole functional group (as defined in more detail below), halogen (F, Cl, Br, I), —CH═C(R³)₂, —C≡CR³, —[C(R³)₂C(R³)₂O]_{q}—R³ wherein q is in the range of 1 to 200, —CN, —N₃, —NCX, —XCN, —XR³, —N(R³)₂, —⁺N(R³)₃, —C(X)N(R³)₂, —C(R³)₂XR³, —C(X)R³, —C(X)XR³, —S(O)R³, —S(O)₂R³, —S(O)OR³, —S(O)₂OR³, —S(O)N(R³)₂, —S(O)₂N(R³)₂, —OS(O)R³, —OS(O)₂R³, —OS(O)OR³, —OS(O)₂OR³, —P(O)(R³)(OR³), —P(O)(OR³)₂, —OP(O)(OR³)₂, —Si(R³)₃, —XC(X)R³, —XC(X)XR³, —XC(X)N(R³)₂, —N(R³)C(X)R³, —N(R³)C(X)XR³, N(R³)C(X)N(R³)₂ and silyl groups wherein the silyl groups can be represented by the formula (R²)₃Si—, wherein R² is independently selected from the group consisting of C₁-C₁₂ alkyl groups, C₂-C₁₂ (hetero)aryl groups, C₃-C₁₂ alkyl(hetero)aryl groups, ₃-C₂₄ (hetero)arylalkyl groups, C₂-C₁₂ alkenyl groups, C₂-C₁₂ alkynyl groups, C₃-C₁₂ cycloalkyl groups, C₁-C₁₂ alkoxy groups, C₂-C₁₂ alkenyloxy groups, C₂-C₁₂ (hetero)aryloxy groups, C₂-C₁₂ alkynyloxy groups and C₃-C₁₂ cycloalkyloxy groups, wherein X is oxygen or sulphur, and wherein R³ is independently selected from the group consisting of hydrogen, halogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, wherein the alkyl groups, cycloalkyl groups, (hetero)aryl groups, alkyl(hetero)aryl groups and (hetero)arylalkyl groups, the alkyl groups, cycloalkyl groups, (hetero)aryl groups, alkyl(hetero)aryl groups, (hetero)arylalkyl groups, alkenyl groups, alkynyl groups, alkoxy groups, alkenyloxy groups, (hetero)aryloxy groups alkynyloxy groups and cycloalkyloxy groups are optionally substituted and optionally interrupted by one or more heteroatoms selected from O, S and N.

When the (hetero)aryl group is substituted with a hydrocarbon substituent, the hydrocarbon substituent may for example be a C₁-C₂₄ alkyl group, a C₃-C₂₄ cycloalkyl group, a C₂-C₂₄ (hetero)aryl group, a C₃-C₂₄ alkyl(hetero)aryl group, a C₃-C₂₄ (hetero)arylalkyl group, a C₁-C₁₂ alkoxy group, a C₃-C₁₂ cycloalkyloxy group, wherein the alkyl group, cycloalkyl group, (hetero)aryl group, alkyl(hetero)aryl group, and (hetero)arylalkyl group, alkoxy group and cycloalkyloxy group is optionally substituted, and wherein the alkyl group, cycloalkyl group, alkyl(hetero)aryl group and (hetero)arylalkyl group are optionally interrupted by one or more heteroatoms selected from the group consisting of O, S and N.

In a preferred embodiment of the process according to the invention, the N-acetylgalactosamine-(hetero)aryl moiety is according to Formula (3a):

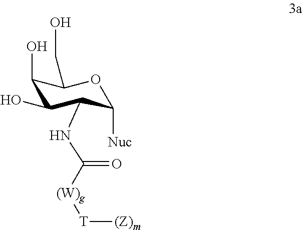

wherein g, T, Nuc and W are as defined above for (2);
m is 0-8; and
Z is a functional group.

The N-acetylgalactosamine-(hetero)aryl moiety, also referred to as Nuc-GalNAryl, according to Formula (3a) comprises 0 to 8 functional groups Z (m is 0-8). In a preferred embodiment of the process according to the invention, m is 0.

In another preferred embodiment of the process according to the invention, m is 1 to 8. In this embodiment, m is preferably 1, 2, 3 or 4, more preferably 1 or 2 and most preferably m is 1.

When m is 2 or more, i.e. when more than 1 functional group Z is present on the (hetero)aryl group T, the functional groups Z are independently selected. In other words, (hetero)aryl group T may be substituted with more than one type of functional group. For example, the (hetero)aryl group may be substituted with a 1,3-dipole functional group, and one or more halogens.

Z is preferably independently selected from the group consisting of a 1,3-dipole functional group, halogen (F, Cl, Br, I), R³, —CH═C(R³)₂, —[C(R³)₂C(R³)₂O]_{q}—R³ wherein q is in the range of 1 to 200, —CN, —NC, NO₂, —NCX, —XCN, —XR³, —N(R³)₂, —⁺N(R³)₃, —C(X)N(R³)₂, —C(R³)₂XR³, —C(X)R³, —C(X)XR³, —S(O)R³, —S(O)₂R³, —S(O)OR³, —S(O)₂OR³, —S(O)N(R³)₂, —S(O)₂N(R³)₂, —OS(O)R³, —OS(O)₂R³, —OS(O)OR³, —OS(O)₂OR³, —P(O)(R³)(OR³), —P(O)(OR³)₂, —OP(O)(OR³)₂, —Si(R³)₃, —XC(X)R³, —XC(X)XR³, —XC(X)N(R³)₂, —N(R³)C(X)R³, —N(R³)C(X)XR³ and —N(R³)C(X)N(R³)₂, wherein X is oxygen or sulphur and wherein R³ is independently selected from the group consisting of hydrogen, halogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups optionally substituted and optionally interrupted by one or more heteroatoms selected from O and N. In this embodiment, it is further preferred that X is O.

Preferably R³ is independently selected from the group consisting of hydrogen, halogen and C₁-C₆ alkyl groups, more preferably from the group consisting of hydrogen, halogen and C₁-C₄ alkyl groups. Most preferably, R³ is independently selected from the group consisting of hydrogen, methyl, ethyl, propyl, i-propyl, butyl and t-butyl. X is preferably oxygen.

More preferably, Z is independently selected from the group consisting of a 1,3-dipole functional group, halogen (F, Cl, Br, I), —CN, —NCX, —XCN, —XR³, —N(R³)₂, —⁺N(R³)₃, —C(X)N(R³)₂, —C(R³)₂XR³, —C(X)R³, —C(X)XR³, —XC(X)R³, —XC(X)XR³, —XC(X)N(R³)₂, —N(R³)C(X)R³, —N(R³)C(X)XR³ and —N(R³)C(X)N(R³)₂, wherein X and R³, and preferred embodiments of X and R³, are as defined above.

Most preferably, Z is selected from the group consisting of a 1,3-dipole functional group, halogen (F, Cl, Br, I), —OR³, —SR³, —N(R³)₂, —⁺N(R³)₃, —C(O)N(R³)₂, —C(O)OR³, —OC(O)R³, —OC(O)OR³, —OC(O)N(R³)₂, —N(R³)C(O)R³, —N(R³)C(O)OR³ and —N(R³)C(O)N(R³)₂, wherein X and R³, and preferred embodiments of X and R³, are as defined above.

When Z is halogen, i.e. Z is F, Cl, Br or I, it is preferred that Z is F, Cl or Br, and preferably F or Cl, and most preferably F.

Optionally, functional group Z is masked or protected.

The term “1,3-dipole functional group” herein refers to a group comprising a three-atom π-electron system containing four electrons delocalized over the three atoms. 1,3-Dipole functional groups are well known in the art.

When Z is a 1,3-dipole functional group, Z is preferably selected from the group consisting of a nitrone group, an azide group, a diazo group, a nitrile oxide group, a nitronate group, a nitrile imine group, a sydnone group, a sulfon hydrazide group, a pyridine oxide group, a oxadiazole 1-oxide group, a dipole group resulting from deprotonation of an alkylated pyridinium compound, a [1,2,3]triazol-8-ium-1-ide group, a 1,2,3-oxadiazol-3-ium-5-olate group and a (hetero)aryl 5-oxopyrazolidin-2-ium-1-ide group.

When Z is a 1,3-dipole functional group, Z is more preferably selected from the group consisting of a nitrone, an azide group, a diazo group, a nitrile oxide group, a nitronate group, a nitrile imine group, a sydnone group, a sulfon hydrazide group, a pyridine oxide group and a oxadiazole 1-oxide group.

When Z is a 1,3-dipole functional group, more preferably Z is selected from the group consisting of a nitrone group, an azide group, a diazo group and a nitrile oxide group, and even more preferably from the group consisting of a nitrone group, an azide group and a nitrile oxide group. When Z is a 1,3-dipole functional group, most preferably Z is an azide group.

The (hetero)aryl group may further comprise additional substituents. These optional additional substituents are preferably independently selected from the group consisting of C₁-C₁₂ alkyl groups, C₂-C₁₂ (hetero)aryl groups, C₂-C₁₂ alkenyl groups, C₂-C₁₂ alkynyl groups, C₃-C₁₂ cycloalkyl groups, C₅-C₁₂ cycloalkenyl groups, C₈-C₁₂ cycloalkynyl groups, C₁-C₁₂ alkoxy groups, C₂-C₁₂ alkenyloxy groups, C₂-C₁₂ (hetero)aryloxy groups, C₂-C₁₂ alkynyloxy groups, C₃-C₁₂ cycloalkyloxy groups, amino groups and silyl groups, wherein the silyl groups can be represented by the formula (R²)₃Si—, wherein R² is independently selected from the group consisting of C₁-C₁₂ alkyl groups, C₂-C₁₂ (hetero)aryl groups, C₂-C₁₂ alkenyl groups, C₂-C₁₂ alkynyl groups, C₃-C₁₂ cycloalkyl groups, C₁-C₁₂ alkoxy groups, C₂-C₁₂ alkenyloxy groups, C₂-C₁₂ (hetero)aryloxy groups, C₂-C₁₂ alkynyloxy groups and C₃-C₁₂ cycloalkyloxy groups, wherein the alkyl groups, (hetero)aryl groups, alkenyl groups, alkynyl groups, cycloalkyl groups, alkoxy groups, alkenyloxy groups, (hetero)aryloxy groups, alkynyloxy groups and cycloalkyloxy groups are optionally substituted, the alkyl groups, the alkoxy groups, the cycloalkyl groups and the cycloalkoxy groups being optionally interrupted by one of more hetero-atoms selected from the group consisting of O, N and S.

In a preferred embodiment of the process according to the invention, the N-acetylgalactosamine-(hetero)aryl moiety is according to Formula (3b):

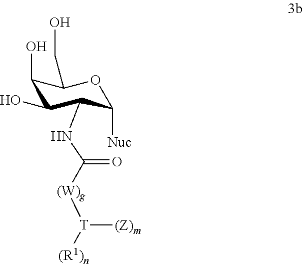

wherein g, T, Nuc and W are as defined above;
n is 0-8;
m is 0-8;
Z is independently selected from the group consisting of functional groups; and
R¹ is independently selected from the group consisting of C₁-C₂₄ alkyl groups, C₂-C₂₄ (hetero)aryl groups, C₃-C₂₄ alkyl(hetero)aryl groups, C₃-C₂₄ (hetero)arylalkyl groups, C₂-C₂₄ alkenyl groups, C₂-C₂₄ alkynyl groups, C₃-C₂₄ cycloalkyl groups, C₅-C₂₄ cycloalkenyl groups, C₈-C₂₄ cycloalkynyl groups, C₁-C₂₄ alkoxy groups, C₂-C₂₄ alkenyloxy groups, C₂-C₂₄ (hetero)aryloxy groups, C₃-C₂₄ alkyl(hetero)aryl groups, C₃-C₂₄ (hetero)arylalkyl groups, C₂-C₂₄ alkynyloxy groups and C₃-C₂₄ cycloalkyloxy groups , wherein the alkyl groups, (hetero)aryl groups, alkyl(hetero)aryl groups, (hetero)arylalkyl groups, alkenyl groups, alkynyl groups, cycloalkyl groups, alkoxy groups, alkenyloxy groups, (hetero)aryloxy groups, alkynyloxy groups and cycloalkyloxy groups are optionally substituted, the alkyl groups, the alkoxy groups, the cycloalkyl groups and the cycloalkoxy groups being optionally interrupted by one of more hetero-atoms selected from the group consisting of O, N and S.

The Nuc-GalNAryl according to Formula (3b) comprises 0 to 8 substituents R¹ (n is 0 to 8). In a preferred embodiment, n is 0. In another preferred embodiment, n is 1, 2, 3 or 4, more preferably n is 1 or 2, and most preferably n is 1. In another preferred embodiment, n is 1, 2, 3, 4, 5, 6, 7 or 8, preferably 1, 2, 3, 4 or 5, more preferably 1, 2, 3 or 4, even more preferably 1, 2 or 3, even more preferably 1 or 2 and most preferably n is 1.

Preferably, R¹ is independently selected from the group consisting of C₁-C₁₂ alkyl groups, C₂-C₁₂ (hetero)aryl groups, C₃-C₁₂ alkyl(hetero)aryl groups, C₃-C₁₂ (hetero)arylalkyl groups, C₂-C₁₂ alkenyl groups, C₂-C₁₂ alkynyl groups, C₃-C₁₂ cycloalkyl groups, C₅-C₁₂ cycloalkenyl groups, C₈-C₁₂ cycloalkynyl groups, C₁-C₁₂ alkoxy groups, C₂-C₁₂ alkenyloxy groups, C₂-C₁₂ (hetero)aryloxy groups, C₃-C₁₂ alkyl(hetero)aryl groups, C₃-C₁₂ (hetero)arylalkyl groups, C₂-C₁₂ alkynyloxy groups, C₃-C₁₂ cycloalkyloxy groups, wherein the alkyl groups, (hetero)aryl groups, alkyl(hetero)aryl groups, (hetero)arylalkyl groups, alkenyl groups, alkynyl groups, cycloalkyl groups, alkoxy groups, alkenyloxy groups, (hetero)aryloxy groups, alkynyloxy groups and cycloalkyloxy groups are optionally substituted, the alkyl groups, the alkoxy groups, the cycloalkyl groups and the cycloalkoxy groups being optionally interrupted by one of more hetero-atoms selected from the group consisting of O, N and S.

More preferably, R¹ is independently selected from the group consisting of C₁-C₁₂ alkyl groups, C₃-C₁₂ cycloalkyl groups, C₂-C₁₂ (hetero)aryl groups, C₃-C₁₂ alkyl(hetero)aryl groups and C₃-C₁₂ (hetero)arylalkyl groups, wherein the alkyl groups, cycloalkyl groups, (hetero)aryl groups, alkyl(hetero)aryl groups and (hetero)arylalkyl groups are optionally substituted, wherein the alkyl groups, cycloalkyl groups, alkyl(hetero)aryl groups and (hetero)arylalkyl groups are optionally interrupted by one or more heteroatoms selected from the group consisting of O, S and N.

Even more preferably, R¹ is independently selected from the group consisting of C₁-C₆ alkyl groups, C₃-C₆ cycloalkyl groups, C₂-C₆ (hetero)aryl groups, C₃-C₆ alkyl(hetero)aryl groups and C₃-C₆ (hetero)arylalkyl groups, wherein the alkyl groups, cycloalkyl groups, (hetero)aryl groups, alkyl(hetero)aryl groups and (hetero)arylalkyl groups are optionally substituted, wherein the alkyl groups, cycloalkyl groups, alkyl(hetero)aryl groups and (hetero)arylalkyl groups are optionally interrupted by one or more heteroatoms selected from the group consisting of O, S and N. Even more preferably, R¹ is independently selected from the group consisting of C₁-C₆ alkyl groups, yet even more preferably R¹ is methyl, ethyl, n-propyl, i-propyl, n-butyl or t-butyl. Most preferably R¹ is methyl, ethyl or i-propyl.

In a preferred embodiment of the process according to the invention, in the Nuc-GalNAryl according to Formula (3b) m is 0, 1, 2, 3, 4, 5, 6, 7 or 8, preferably 0, 1, 2, 3, 4 or 5, more preferably 0, 1, 2, 3 or 4, even more preferably 0, 1, 2 or 3, even more preferably 0, 1 or 2 and most preferably m is 0 or 1.

In a preferred embodiment of (3b), n is 0, 1, 2, 3, 4, 5, 6, 7 or 8, preferably 0, 1, 2, 3, 4 or 5, more preferably 0, 1, 2, 3 or 4, even more preferably 0, 1, 2 or 3, even more preferably 0, 1 or 2 and most preferably n is 0 or 1.

In a preferred embodiment of the process according to the invention, in the Nuc-GalNAryl according to Formula (3b) m is 1, 2, 3, 4, 5, 6, 7 or 8, preferably 1, 2, 3, 4 or 5, more preferably 1, 2, 3 or 4, even more preferably 1, 2 or 3, even more preferably 1 or 2 and most preferably m is 1.

In a preferred embodiment of (3b), n is 1, 2, 3, 4, 5, 6, 7 or 8, preferably 1, 2, 3, 4 or 5, more preferably 1, 2, 3 or 4, even more preferably 1, 2 or 3, even more preferably 1 or 2 and most preferably n is 1.

In a preferred embodiment of the process according to the invention, in the Nuc-GalNAryl according to Formula (3b) m is 0 and n is 0. In other words, in this preferred embodiment no substituents are present on (hetero)aryl group T.

However, when (hetero)aryl group T in (3b) is phenyl and g is 0 (i.e. W is absent), it is preferred that m is 0, 1, 2, 3, 4, 5, 6, 7 or 8 (preferably 0, 1, 2, 3, 4 or 5), and n is 0, 1, 2, 3, 4, 5, 6, 7 or 8 (preferably 0, 1, 2, 3, 4 or 5), with the proviso that m and n are not both 0. Also when (hetero)aryl group T in (3b) is phenyl and g is 1 (i.e. W is present), it is preferred that m is 0, 1, 2, 3, 4, 5, 6, 7 or 8 (preferably 0, 1, 2, 3, 4 or 5), and n is 0, 1, 2, 3, 4, 5, 6, 7 or 8 (preferably 0, 1, 2, 3, 4 or 5), with the proviso that m and n are not both 0.

In another preferred embodiment of the process according to the invention, in the Nuc-GalNAryl according to Formula (3b) m is 1 to 8, preferably 1, 2, 3, 4 or 5, and n is 0. In this embodiment is further preferred that m is 1, 2, 3 or 4 and n is 0, more preferably m is 1, 2 or 3 and n is 0, yet more preferably m is 1 or 2 and n is 0, and most preferably m is 1 and n is 0.

In another preferred embodiment, m is 0 and n is 1, 2, 3, 4 or 5, preferably m is 0 and n is 1, 2, 3 or 4. More preferably, m is 0 and n is 1, 2 or 3. Even more preferably m is 0 and n is 1 or 2, and most preferably m is 0 and n is 1.

In Nuc-GalNAryl according to Formula (3a) and (3b) it is preferred that Nuc is UDP. The (hetero)aryl group in Nuc-GalNAryl according to Formula (3a) and (3b) may be any aryl group or any heteroaryl group. Preferably, the (hetero)aryl group is as defined above for Nuc-GalNAryl according to Formula (2). In a further preferred embodiment, the (hetero)aryl group is selected from the group consisting of phenyl groups, pyridinyl groups, pyridiniumyl groups, pyrimidinyl groups, pyrimidinium groups, pyrazinyl groups, pyradizinyl groups, pyrrolyl groups, pyrrolium groups, furanyl groups, thiophenyl groups (i.e. thiofuranyl groups), diazolyl groups, quinolinyl groups, imidazolyl groups, oxazolyl groups and oxazolium groups.

In a preferred embodiment of the process according to the invention, the N-acetylgalactosamine-(hetero)aryl moiety Nuc-GalNAryl is according to Formula (4a), (4b), (4c), (4d), (4e), or (4f):

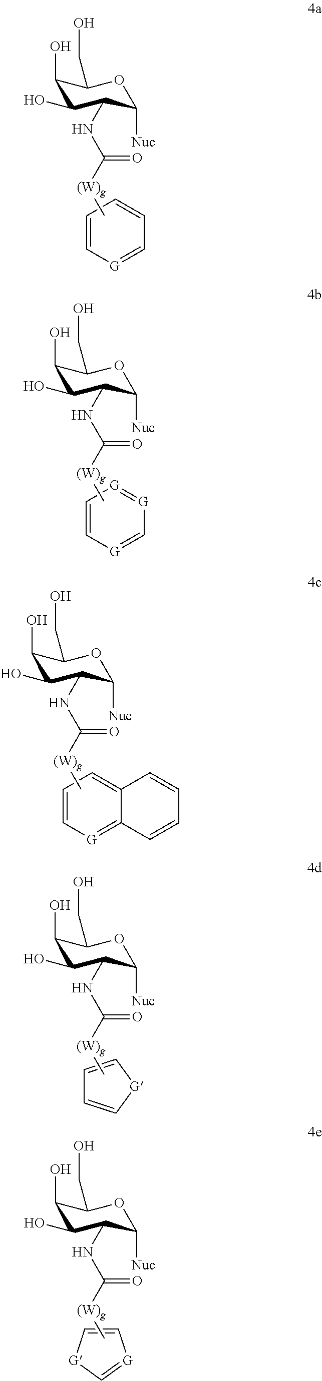

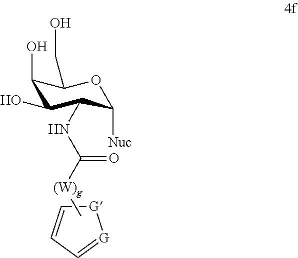

wherein:
Nuc, W and g are as defined above;
G is independently selected from the group consisting of N, CH, CR⁴, CR⁵, CZ, and N⁺R⁴, wherein R⁴ is selected from the group consisting of C₁-C₂₄ alkyl groups and wherein R⁵ is selected from the group consisting of hydrogen, R¹ and R⁴;
G′ is independently selected from the group consisting of O, S, NR⁵ and N⁺(R⁴)₂, wherein R⁴ and R⁵ are as defined above and R¹ is as defined above for Nuc-GalNAryl (3).

Preferably, G is selected from the group consisting of N, CH, CZ, CR⁵ and N⁺R⁴ and G′ is selected from the group consisting of O, S, NR⁵ and N⁺(R⁴)₂, wherein R⁴ and R⁵ are as defined above.

In (4a), (hetero)aryl group T may e.g. be phenyl, pyridinyl or pyridiniumyl. In (4b), (hetero)aryl group T may e.g. be pyrazinyl, pyradizinyl, pyrimidinyl, pyrimidiniumyl, or triazinyl. In (4c), (hetero)aryl group T may e.g. be quinolinyl. In (4d), (hetero)aryl group T may for example be pyrrolyl, pyrrolium, pyrrolidiniumyl, furanyl or thiophenyl (i.e. thiofuranyl). In (4e), (hetero)aryl group T may for example be diazolyl, oxazolyl, imidazolyl or thiazolyl. In (4f), (hetero)aryl group T may for example be pyrazolyl, isoxathiazolyl, isoazathiazolyl or isoxazolyl.

Also in Nuc-GalNAryl (4a), (4b), (4c), (4d), (4e) and (4f) it is preferred that Nuc is UDP. The (hetero)aryl group in Nuc-GalNAryl (4a)-(4f) may be any aryl group or any heteroaryl group, and is optionally substituted with one or more substituents as described in more detail above for GalNAryl (2). Preferably, the (hetero)aryl group is as defined above for Nuc-GalNAryl (2). In a further preferred embodiment, the (hetero)aryl group is selected from the group consisting of phenyl groups, pyridinyl groups, pyridiniumyl groups, pyrimidinyl groups, pyrimidinium groups, pyrazinyl groups, pyradizinyl groups, pyrrolyl groups, pyrrolium groups, furanyl groups, thiophenyl groups (i.e. thiofuranyl groups), diazolyl groups, quinolinyl groups, imidazolyl groups, oxazolyl groups and oxazolium groups.

In a preferred embodiment of the process according to the invention, the N-acetylgalactosamine-(hetero)aryl moiety Nuc-GalNAryl is according to Formula (5a), (5b), (5c), (5d), (5e), or (5f):

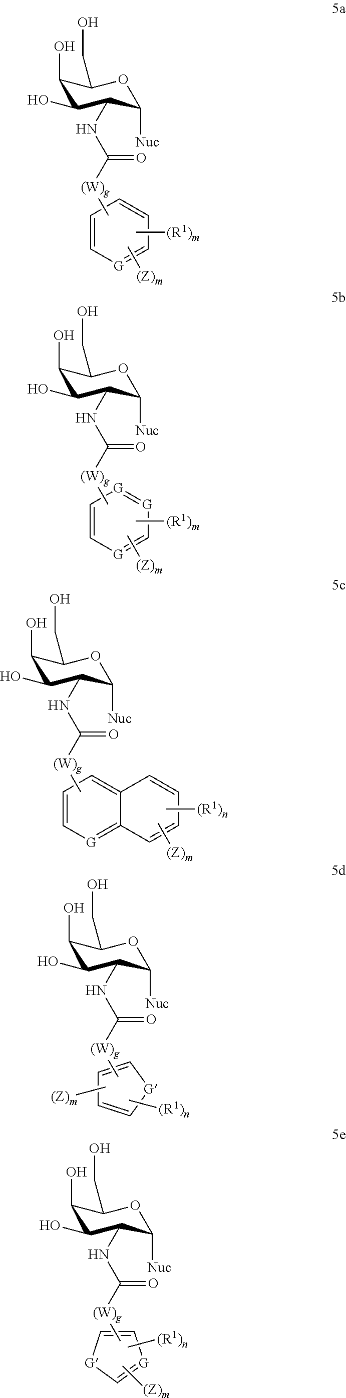

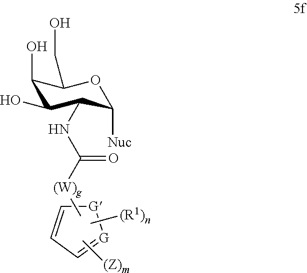

wherein:
Nuc, W, g, R¹, Z, m and n are as defined above for GalNAryl (3b); and
G and G′ are as defined above for Nuc-GalNAryl (4a)-(4f).

In (4a)-(4f) and (5a)-(5f) it is preferred that Nuc is UDP. Furthermore, in (4a) and (5a) the (hetero)aryl group is preferably selected from the group consisting of phenyl groups, pyridinyl groups and pyridiniumyl groups. In (4b) and (5b) the (hetero)aryl group is preferably selected from the group consisting of pyrazinyl, pyradizinyl, pyrimidinyl, pyrimidiniumyl and triazinyl groups. In (4c) and (5c), the (hetero)aryl group is preferably selected from the group consisting of quinolinyl groups. In (4d) and (5d) the (hetero)aryl group is preferably selected from the group consisting of pyrrolyl, pyrrolium, pyrrolidiniumyl, furanyl or thiophenyl (i.e. thiofuranyl) groups. In (4e) and (5e) the (hetero)aryl group is preferably selected from the group consisting of diazolyl, oxazolyl, imidazolyl or thiazolyl groups. In (4f) and (5f) the (hetero)aryl group is preferably selected from the group consisting of pyrazolyl or isoxazolyl groups.

In a preferred embodiment of the process according to the invention, T is a pyridinyl group, and the N-acetylgalactosamine-(hetero)aryl moiety Nuc-GalNAryl is according to Formula (21b), preferably (21):

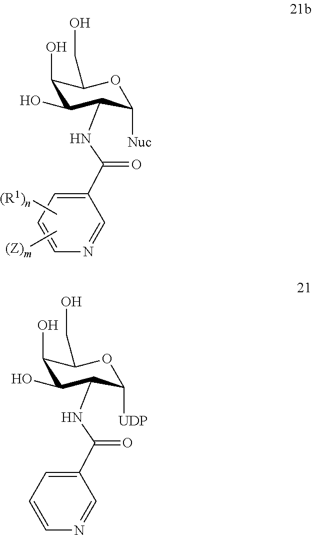

In (21), m and n are all 0. Nuc is UDP in (21). In (21b), it is also preferred that Nuc is UDP. In (21b) it is further preferred that m is 0 or 1. Preferably n is 0, 1 or 2. More preferably, n is 1. In another preferred embodiment, m is 0. When m is 1, Z is a functional group as defined above.

In another preferred embodiment of the process according to the invention, T is a pyridinyl group, and the N-acetylgalactosamine-(hetero)aryl moiety Nuc-GalNAryl is according to Formula (21c), (21d) or (21e):

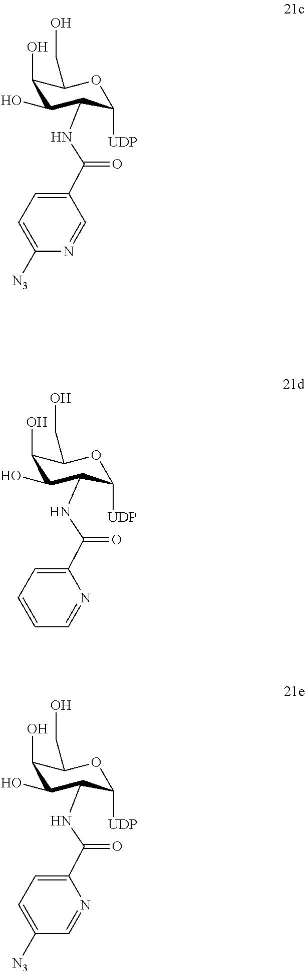

In another preferred embodiment of the process according to the invention, the N-acetylgalactosamine-(hetero)aryl moiety Nuc-GalNAryl is according to Formula (22) or (22b):

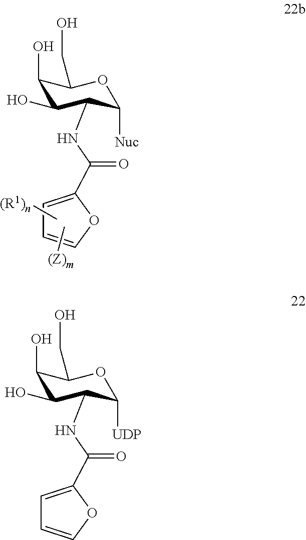

In (22), n and m are 0. Nuc is UDP in (22). In (22b), it is also preferred that Nuc is UDP. In (22b) it is further preferred that m is 0 or 1. Preferably n is 0 or 1. More preferably, m is 1 and n is 0, or m is 0 and n is 1, or m and n are 1.

When the Nuc-GalNAryl is according to Formula (21b) or (22b), m is 0, 1, 2, 3, 4, 5, 6, 7 or 8, preferably 0, 1, 2, 3 or 4, more preferably 0, 1, 2 or 3, even more preferably 0, 1 or 2 and most preferably m is 0 or 1.

In a preferred embodiment of (21b) and (22b), n is 0, 1, 2, 3, 4, 5, 6, 7 or 8, preferably 0, 1, 2, 3 or 4, more preferably 0, 1, 2 or 3, even more preferably 0, 1 or 2 and most preferably n is 0 or 1.

In a preferred embodiment of (21b) and (22b), m is 1, 2, 3, 4, 5, 6, 7 or 8, preferably 1, 2, 3 or 4, more preferably 1, 2 or 3, even more preferably 1 or 2 and most preferably m is 1.

In a preferred embodiment of (21b) and (22b), n is 1, 2, 3, 4, 5, 6, 7 or 8, preferably 1, 2, 3 or 4, more preferably 1, 2 or 3, even more preferably 1 or 2 and most preferably n is 1.

In a preferred embodiment of (21b) and (22b), m is 0 and n is 0. In other words, in this preferred embodiment no substituents are present on (hetero)aryl group T.

In another preferred embodiment of the process according to the invention, in (21b) and (22b), m is 1 to 8 (preferably 1, 2, 3, 4 or 5), and n is 0. In this embodiment is further preferred that m is 1, 2, 3 or 4 and n is 0, more preferably m is 1, 2 or 3 and n is 0, yet more preferably m is 1 or 2 and n is 0, and most preferably m is 1 and n is 0.

In another preferred embodiment of (21b) and (22b), m is 0 and n is 1, 2, 3, 4 or 5, preferably m is 0 and n is 1, 2, 3 or 4. More preferably, m is 0 and n is 1, 2 or 3. Even more preferably m is 0 and n is 1 or 2, and most preferably m is 0 and n is 1.

In another preferred embodiment of the process according to the invention, the N-acetylgalactosamine-(hetero)aryl moiety Nuc-GalNAryl is according to Formula (23) or (23b):

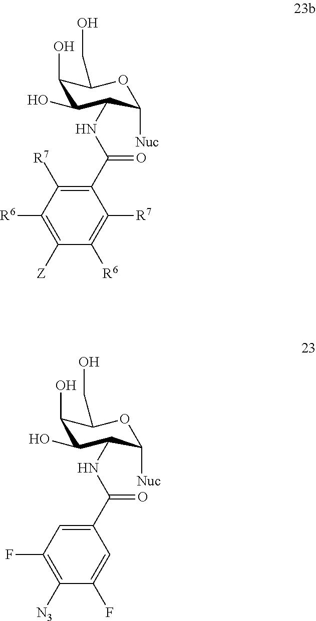

wherein:
Nuc is a nucleotide;
Z is a functional group;
R⁶ is independently selected from the group consisting of hydrogen, F, Cl, Br and I; and
R⁷ is independently selected from the group consisting of hydrogen, F, Cl, Br and I.

In (23b), it is preferred that R⁶ is independently selected from the group consisting of hydrogen, F and Cl, and preferably R⁶ is hydrogen or F. In (23b), it is preferred that R⁷ is independently selected from the group consisting of hydrogen, F and Cl, and preferably R⁷ is hydrogen or F.

In (23), m is 1 and Z is an azide group. Nuc is UDP in (23). In (23b), it is also preferred that Nuc is UDP. In (23b), R⁶ is F or Cl, and R⁷ is H, F or Cl. It is further preferred that both R⁶ groups are identical to each other, and that both R⁷ groups are identical to each other. In a particularly preferred embodiment, both R⁶ groups are Cl, both R⁷ groups are H and Nuc is UDP. In this embodiment it is further preferred that Z is an azide group. The fluorinated counterpart of this particularly preferred embodiment is (23). In another particularly preferred embodiment, R⁶ and R⁷ are all the same, i.e. the phenyl group in (23) preferably comprises four identical substituents in addition to Z. In a particularly preferred embodiment, R⁶ and R⁷ are F. In this embodiment it is further preferred that Z is an azide group and that Nuc is UDP. Most preferably, R⁶ and R⁷ are F, Z is an azide group and Nuc is UDP. In yet another particularly preferred embodiment, R⁶ and R⁷ are Cl. In this embodiment it is further preferred that Z is an azide group and that Nuc is UDP. Most preferably, R⁶ and R⁷ are Cl, Z is an azide group and Nuc is UDP. In another preferred embodiment of the process according to the invention, the N-acetylgalactosamine-(hetero)aryl moiety Nuc-GalNAryl is according to Formula (23), (23c), 23d) or (23e):

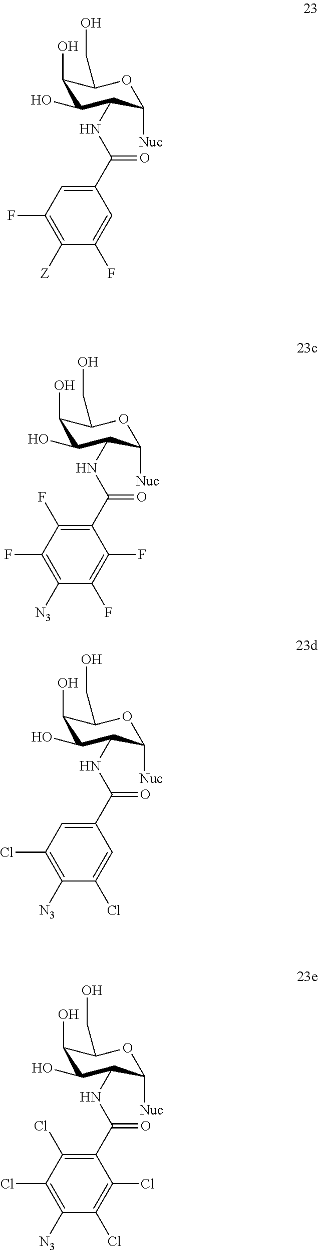

Wherein Nuc is a nucleotide, as described in more detail above. Preferably, Nuc is UDP.

The synthesis method of UDP-GalNAryl according to Formula (21), (22) and (23) is shown schematically in FIG. 5.

### (A-L)-GlcNAc Moiety

In the process according to the invention, the N-acetylglucosamine moiety is according to Formula (1):

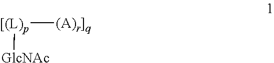

wherein p, q, r, L and A are as defined above.

The N-acetylglucosamine moiety according to Formula (1) is herein also referred to as (A-L)-GlcNAc. (A-L)-GlcNAc is composed of a GlcNAc sugar, optionally (q is 0 or 1) substituted with (L)ₚ-(A)ᵣ. Linking units L and moieties A are described in more detail below.

When q is 0, the GlcNAc moiety does not comprise a substituent (L)ₚ-(A)ᵣ, and in this case the GlcNAc moiety (1) is unsubstituted GlcNAc (N-acetylglucosamine).

When q is 1, then a substituent (L)ₚ-(A)ᵣ is present in the GlcNAc moiety. In this embodiment, one or more moieties A are present in the GlcNAc moiety.

The substituent (L)ₚ-(A)ᵣ is present on the C1 carbon atom of the GlcNAc in the GlcNAc moiety. When a linker L is present (p is 1), up to 4 moieties A may be linked via linker L to the GlcNAc in the GlcNAc moiety (r is 1, 2, 3 or 4).

When q is 1 and p is 0, i.e. linker L is absent, one moiety A is present in the GlcNAc moiety, and A is directly bonded to the C1 carbon atom of GlcNAc. In this case, A is bonded to C1 via an O-atom, an N-atom or a C-atom, preferably via an O- or an N-atom, most preferably via an O-atom. When A is bonded via an O-atom, it is further preferred that the O-atom is the O-atom of the OH-group of GlcNAc, in other words A then preferably replaces the H-atom of said OH-group. When A is bonded via an N- or a C-atom, the N- or C-atom, which may be further substituted, preferably replaces the OH-group on the C1 carbon atom of GlcNAc.

When a linker L is present (p is 1), (L)ₚ-(A)ᵣ is bonded to the C1 carbon atom of GlcNAc via an O-atom, an N-atom or a C-atom, preferably via an O-atom or an N-atom, and most preferably via an O-atom. When (L)ₚ-(A)ᵣ is bonded via an O-atom, it is preferred that the O-atom is the O-atom of the OH-group of GlcNAc, in other words (L)ₚ-(A)ᵣ then preferably replaces the H-atom of said OH-group. When (L)ₚ-(A)ᵣ is bonded via an N- or a C-atom, the N- or C-atom preferably replaces the OH-group of GlcNAc. In this case, linker L may be —N(R⁸)— or —C(R⁸)₂—, or alternatively the N- or C-atom may be part of a larger linker L.

As was described above, when a linker L is present, up to 4 moieties A may be present in GlcNAc moiety A (r is 1, 2, 3 or 4). Preferably, r is 1 or 2, and more preferably r is 1. When more than 1 moiety is present in (A)-(L)-GlcNAc (r is 2, 3 or 4), each A is selected independently.

A is selected independently from the group consisting of D, E and Q, wherein D is a molecule of interest, E is a solid surface and Q is a functional group. Molecules of interest D, solid surfaces E and functional groups Q are described in more detail below.

A molecule of interest D may for example be a reporter molecule, a diagnostic compound, an active substance, an enzyme, an amino acid (including an unnatural amino acid), a (non-catalytic) protein, a peptide, a polypeptide, an oligonucleotide, a monosaccharide, an oligosaccharide, a polysaccharide, a glycan, a (poly)ethylene glycol diamine (e.g. 1,8-diamino-3,6-dioxaoctane or equivalents comprising longer ethylene glycol chains), a polyethylene glycol chain, a polyethylene oxide chain, a polypropylene glycol chain, a polypropylene oxide chain or a 1,x-diaminoalkane (wherein x is the number of carbon atoms in the alkane).

An active substance is a pharmacological and/or biological substance, i.e. a substance that is biologically and/or pharmaceutically active, for example a drug or a prodrug, a diagnostic agent, an amino acid, a protein, a peptide, a polypeptide, a monosaccharide, an oligosaccharide, a polysaccharide, a glycan, a lipid, a vitamin, a steroid, a nucleotide, a nucleoside, a polynucleotide, RNA or DNA. Examples of suitable peptide tags include a cell-penetrating peptide like human lactoferrin or polyarginine. An example of a suitable glycan is oligomannose.

Preferably, the active substance is selected from the group consisting of drugs and prodrugs. More preferably, the active substance is selected from the group consisting of pharmaceutically active compounds, in particular low to medium molecular weight compounds (e.g. about 200 to about 1500 Da, preferably about 300 to about 1000 Da), such as for example cytotoxins, antiviral agents, antibacterials agents, peptides and oligonucleotides. Examples of cytotoxins include colchicine, vinca alkaloids, camptothecins, doxorubicin, daunorubicin, taxanes, calicheamycins, duocarmycins, maytansines, auristatins, tubuly sin, irinotecans, an inhibitory peptide, amanitin, deBouganin, or pyrrolobenzodiazepines (PBDs). In a preferred embodiment, the cytotoxin is selected from the group consisting of camptothecins, doxorubicin, daunorubicin, taxanes, calicheamycins, duocarmycins, maytansines, auristatins and pyrrolobenzodiazepines (PBDs). In another preferred embodiment, the cytotoxin is selected from the group consisting of colchicine, vinca alkaloids, tubulysins, irinotecans, an inhibitory peptide, amanitin and deBouganin.

A reporter molecule is a molecule whose presence is readily detected, for example a diagnostic agent, a dye, a fluorophore, a radioactive isotope label, a contrast agent, a magnetic resonance imaging agent or a mass label. Examples of a fluorophore include all kinds of Alexa Fluor (e.g. Alexa Fluor 555), cyanine dyes (e.g. Cy3 or Cy5), coumarin and coumarin derivatives, fluorescein, rhodamine, allophycocyanin and chromomycin.

Examples of radioactive isotope label include ^{99m}Tc, ¹¹¹In, ¹⁸F, ⁶⁸Ga, ¹¹C, ⁶⁴Cu, ¹³¹I or ¹²³I, which may or may not be connected via a chelating moiety such as DTPA, DOTA, NOTA or HYNIC.

A solid surface E is for example a functional surface (e.g. nanomaterials, carbon nanotubes, fullerenes, virus capsids), a metal surface (e.g. gold, silver, copper, nickel, tin, rhodium, zinc) or a metal alloy surface (from aluminium, bismuth, chromium, cobalt, copper, gallium, gold, indium, iron, lead, magnesium, mercury, nickel, potassium, plutonium, rhodium, scandium, silver, sodium, titanium, tin, uranium, zinc, zirconium), a polymer surface (e.g. polystyrene, polyvinylchloride, polyethylene, polypropylene, poly(dimethylsiloxane), polymethylmethacrylate, polyisocyanate). E is preferably independently selected from the group consisting of a functional surface or a polymer surface.

In a preferred embodiment of the process according to the invention, A is a molecule of interest D. More preferably, A is independently selected from the group consisting of a reporter molecule, an active substance, an enzyme, a protein, a glycoprotein, an antibody, a peptide, a polypeptide, an oligonucleotide, a monosaccharide, an oligosaccharide, a polysaccharide, a glycan, a diagnostic compound, an amino acid, a (poly)ethylene glycol diamine, a polyethylene glycol chain, a polyethylene oxide chain, a polypropylene glycol chain, a polypropylene oxide chain and a 1,x-diaminoalkane (wherein x is the number of carbon atoms in the alkane). Reporter molecules and active substances are described in more detail above.

In a particularly preferred embodiment, A is a glycoprotein, more preferably an N-glycoprotein, most preferably an antibody, as described in more detail below.

When A is a functional group Q, A is preferably selected from the group consisting of hydrogen, halogen, R³, —CH═C(R³)₂, —C≡CR³, —[C(R³)₂C(R³)₂O]_{q}—R³ wherein q is in the range of 1 to 200, —CN, —N₃, —NCX, —XCN, —XR³, —N(R³)₂, —⁺N(R³)₃, —C(X)N(R³)₂, —C(R³)₂XR³, —C(X)R³, —C(X)XR³, —S(O)R³, —S(O)₂R³, —S(O)OR³, —S(O)₂OR³, —S(O)N(R³)₂, —S(O)₂N(R³)₂, —OS(O)R³, —OS(O)₂R³, —OS(O)OR³, —OS(O)₂OR³, —P(O)(R³)(OR³), —p(O)(OR³)₂, —OP(O)(OR³)₂, —Si(R³)₃, —XC(X)R³, —XC(X)XR³, —XC(X)N(R³)₂, —N(R³)C(X)R³, —N(R³)C(X)XR³ and —N(R³)C(X)N(R³)₂, wherein X is oxygen or sulphur and wherein R³ is independently selected from the group consisting of hydrogen, halogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups optionally substituted and optionally interrupted by one or more heteroatoms selected from O and N.

Preferably R³ is independently selected from the group consisting of hydrogen, halogen and C₁-C₆ alkyl groups, more preferably from the group consisting of hydrogen, halogen and C₁-C₄ alkyl groups. Most preferably, R³ is independently selected from the group consisting of hydrogen, methyl, ethyl, propyl, i-propyl, butyl and t-butyl. X is preferably oxygen.

Optionally, functional group Q is masked or protected. More preferably, Q is independently selected from the group consisting of —CN, —NCX, —XCN, —XR³, —N(R³)₂, —⁺N(R³)₃, —C(X)N(R³)₂, —C(R³)₂XR³, —C(X)R³, —C(X)XR³, —XC(X)R³, —XC(X)XR³, —XC(X)N(R³)₂, —N(R³)C(X)R³, —N(R³)C(X)XR³ and —N(R³)C(X)N(R³)₂, wherein X and R³, and preferred embodiments of X and R³, are as defined above. Most preferably, Q is selected from the group consisting of —OR³, —SR³, —N(R³)₂, —⁻N(R³)₃, —C(O)N(R³)₂, —C(O)OR³, —OC(O)R³, —OC(O)OR³, —OC(O)N(R³)₂, —N(R³)C(O)R³, —N(R³)C(O)OR³ and —N(R³)C(O)N(R³)₂, wherein X and R³, and preferred embodiments of X and R³, are as defined above.

When p is 1, a linker L is present in the GlcNAc moiety. The linker, if present, covalently attaches A to the GlcNAc present in (1). Linkers L, also referred to as linking units, are well known in the art. In a GlcNAc moiety as described herein, L, if present, is linked to a moiety A as well as to C1 of the GlcNac in (L)-(A), as was described above. Numerous methods for linking C1 of said GlcNAc and moiety A to L are known in the art. As will be clear to a person skilled in the art, the choice of a suitable method for linking a GlcNAc moiety to one end of a linker and a moiety A to another end depends on the exact nature of the GlcNAc moiety, the linker and the molecule of interest.

A linker may have the general structure F¹-L(F²)ᵣ, wherein F¹ represents a functional group that is able to react with the OH group present on C1 GlcNAc in the GlcNAc moiety. F² represents a functional group that is able to react with a functional group F on moiety A.

Since more than one moiety A may be bonded to a linker, more than one functional group F² may be present on L. As was described above, r is 1, 2, 3 or 4, more preferably r is 1 or 2 and most preferably r is 1.

L may for example be selected from the group consisting of linear or branched C₁-C₂₀₀ alkylene groups, C₂-C₂₀₀ alkenylene groups, C₂-C₂₀₀ alkynylene groups, C₃-C₂₀₀ cycloalkylene groups, C₅-C₂₀₀ cycloalkenylene groups, C₈-C₂₀₀ cycloalkynylene groups, C₇-C₂₀₀ alkylarylene groups, C₇-C₂₀₀ arylalkylene groups, C₈-C₂₀₀ arylalkenylene groups, C₉-C₂₀₀ arylalkynylene groups. Optionally the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups may be substituted, and optionally said groups may be interrupted by one or more heteroatoms, preferably 1 to 100 heteroatoms, said heteroatoms preferably being selected from the group consisting of O, S and NR³, wherein R³ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups. Most preferably, the heteroatom is O.

F, F¹ and F² may for example be independently selected from the group consisting of hydrogen, halogen, R³, C₄-C₁₀ (hetero)cycloalkyne groups, —CH═C(R³)₂, —C≡CR³, —[C(R³)₂C(R³)₂O]_{q}—R³, wherein q is in the range of 1 to 200, —CN, —N₃, —NCX, —XCN, —XR³, —N(R³)₂, —⁺N(R³)₃, —C(X)N(R³)₂, —C(R³)₂XR³, —C(X)R³, —C(X)XR³, —S(O)R³, —S(O)₂R³, —S(O)OR³, —S(O)₂OR³, —S(O)N(R³)₂, —S(O)₂N(R³)₂, —OS(O)R³, —OS(O)₂R³, —OS(O)OR³, —OS(O)₂OR³, —P(O)(R³)(OR³), —P(O)(OR³)₂, —OP(O)(OR³)₂, —Si(R³)₃, —XC(X)R³, —XC(X)XR³, —XC(X)N(R³)₂, —N(R³)C(X)R³, —N(R³)C(X)XR³ and —N(R³)C(X)N(R³)₂, wherein X is oxygen or sulphur and wherein R³ is as defined above.

Examples of suitable linking units include (poly)ethylene glycol diamines (e.g. 1,8-diamino-3,6-dioxaoctane or equivalents comprising longer ethylene glycol chains), polyethylene glycol or polyethylene oxide chains, polypropylene glycol or polypropylene oxide chains and 1,x-diaminoalkanes wherein x is the number of carbon atoms in the alkane.

Another class of suitable linkers comprises cleavable linkers. Cleavable linkers are well known in the art. For example Shabat et al., Soft Matter 2012, 6, 1073, incorporated by reference herein, discloses cleavable linkers comprising self-immolative moieties that are released upon a biological trigger, e.g. an enzymatic cleavage or an oxidation event. Some examples of suitable cleavable linkers are peptide-linkers that are cleaved upon specific recognition by a protease, e.g. cathepsin, plasmin or metalloproteases, or glycoside-based linkers that are cleaved upon specific recognition by a glycosidase, e.g. glucoronidase, or nitroaromatics that are reduced in oxygen-poor, hypoxic areas.

As was described in more detail above, Moiety A may also be bonded to C1 of the GlcNAc in the GlcNAc moiety via an N-atom, an O-atom or a C-atom. If this is the case, then said N-atom, an O-atom or a C-atom may herein also be considered a linker. Linker L may thus also be selected from the group consisting of —O—, —N(R⁸)— and —C(R⁸)₂—, wherein R⁸ is selected from the group consisting of hydrogen and C₁-C₁₂ alkyl groups, more preferably from the group consisting of hydrogen and C₁-C₆ alkyl groups, even more preferably from the group consisting of hydrogen and C₁-C₄ alkyl groups. In this embodiment, L is preferably —O—, —CH₂—, —C(Me)₂—, —NH— or —NMe₂—.

An example of a GlcNAc moiety in the process according to the invention is N-acetylglucosamine moiety (25):

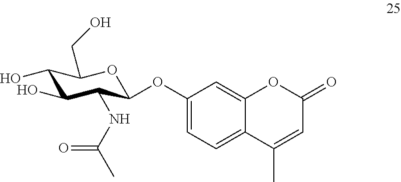

In GlcNAc moiety (25), L is an O-atom, and A is coumarin.

When the GlcNAc of GlcNAc moiety (25) is connected to the GalNAryl moiety of e.g. UDP-GalNAryl (22), via the process according to the invention, a product according to Formula (26) is obtained:

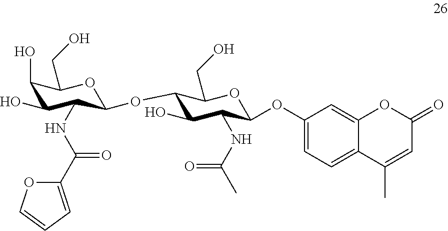

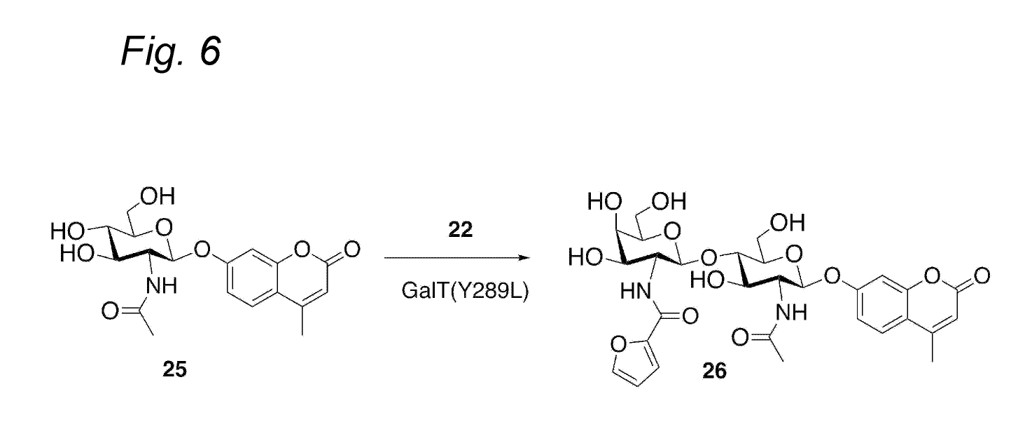

The process for the connection of the GlcNAc of GlcNAc moiety (25) and the GalNAryl moiety of e.g. UDP-GalNAryl (22) is shown in FIG. 6.

In a preferred embodiment of the process according to the invention, the N-acetylgalactosamine-(hetero)aryl moiety is according to Formula (2) as defined above, and the (hetero)aryl group is substituted with a functional group. In a further preferred embodiment, said functional group is a 1,3-dipole functional group, as described above.

In another preferred embodiment of the process according to the invention, the N-acetylgalactosamine-(hetero)aryl moiety is according to Formula (3a) or (3b) as defined above. In this embodiment, it is further preferred that m is 1 and Z is a 1,3-dipole functional group.

In a preferred embodiment, the GlcNAc in N-acetylglucosamine moiety (1) is a terminal GlcNAc moiety of a glycoprotein glycan. In this embodiment it is further preferred that the N-acetylglucosamine moiety (1) is according to Formula (10) or (11):

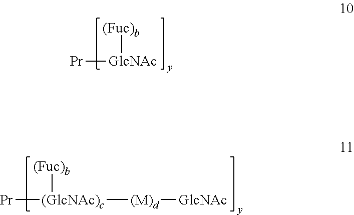

wherein:
y is 1-20;
b is 0 or 1;
c is 0 or 1;
d is 0 or 1;
Pr is a glycoprotein; and
M is a monosaccharide, or a linear or branched oligosaccharide comprising 2 to 20 saccharide moieties.

In this embodiment of the process according to the invention, the GlcNAc in GlcNAc moiety (1) is the terminal GlcNAc of a glycoprotein glycan, i.e. in this embodiment A in GlcNAc moiety (1) is a glycoprotein. A “terminal GlcNAc” is herein defined as a Glc-NAc moiety that is present at the non-reducing end of the glycan.

M is a linear or branched oligosaccharide, and preferably M comprises 2 to 12, more preferably 2 to 10, even more preferably 2 to 8 and most preferably 2 to 6 sugar moieties. Sugar moieties that may be present in a glycan are known to a person skilled in the art, and include e.g. glucose (Glc), galactose (Gal), mannose (Man), fucose (Fuc), N-acetylglucosamine (GlcNAc), N-acetylgalactosamine (GalNAc), N-acetylneuraminic acid (NeuNAc) or sialic acid, and xylose (Xyl).

Preferably, when c is 0 then d is 1, and when d is 0 then c is 1.

In a preferred embodiment of the process according to the invention, the glycan on the glycoprotein consists of one GlcNAc, and the glycoprotein is according to formula (10), wherein b is 0. In another preferred embodiment, said glycan consists of a fucosylated GlcNAc, and the glycoprotein is according to formula (10), wherein b is 1. The GlcNAc of a glycan according to formula (10) wherein b is 1, is herein also considered a terminal GlcNAc.

In yet another preferred embodiment, said glycoprotein is according to formula (11), wherein the core-GlcNAc, if present, is optionally fucosylated (b is 0 or 1). When a core-GlcNAc is fucosylated, fucose is most commonly linked α-1,6 to C6 of the core-GlcNAc.

In this embodiment of the process according to the invention, a glycoprotein mixture may be used as the starting glycoprotein, said mixture comprising glycoproteins comprising one or more fucosylated (b is 1) glycans and/or one or more non-fucosylated (b is 0) glycans.

A glycoprotein comprising a glycan comprising a terminal GlcNAc is herein also referred to as a “terminal non-reducing GlcNAc-protein”, and a glycan comprising a terminal GlcNAc is herein also referred to as a “terminal non-reducing GlcNAc-glycan”. It should be noted that the term “terminal non-reducing GlcNAc-protein” includes a glycoprotein of formula (10) wherein b is 1.

The terminal non-reducing GlcNAc-protein may comprise a linear or a branched terminal non-reducing GlcNAc-glycan. Said glycan is bonded via C1 of the core-sugar to the protein, and said core-sugar preferably is a core-GlcNAc or a core-GalNAc, more preferably a core-GlcNAc. Therefore, when the glycoprotein is according to formula (11), it is preferred that c is 1.

In a preferred embodiment, C1 of the core-sugar of the terminal non-reducing GlcNAc-glycan is bonded to the glycoprotein via an N-glycosidic bond to a nitrogen atom in an amino acid residue in said protein, more preferably to an amide nitrogen atom in the side chain of an asparagine (Asn) or an arginine (Arg) amino acid. However, C1 of the core-sugar of the non-reducing GlcNAc-glycan may also be bonded to the protein via an O-glycosidic bond to an oxygen atom in an amino acid residue in said protein, more preferably to an oxygen atom in the side chain of a serine (Ser) or threonine (Thr) amino acid. In this embodiment, it is preferred that the core-sugar of said glycan is an O-GlcNAc or an O-GalNAc, preferably an O-GlcNAc. C1 of the core-sugar of the non-reducing GlcNAc-glycan may also be bonded to the protein via a C-glycosidic bond to a carbon atom on the protein, e.g. to tryptophan (Trp).

A glycoprotein according to Formula (10) or (11) may comprise more than one glycan (y is 1-20), and may comprise a combination of N-linked, O-linked and C-linked glycans. Preferably, y is 1 to 12, more preferably y is 1, 2, 3, 4, 5, 6, 7 or 8, and even more preferably y is 1, 2, 3 or 4. Most preferably y is 1 or 2.

In yet another preferred embodiment, y is 2, 4, 6 or 8, preferably 2 or 4, most preferably 2. This embodiment is particularly preferred when the glycoprotein is an antibody (Ab), i.e. when Pr is Ab, as described in more detail below.

The terminal non-reducing GlcNAc-glycan may be present at a native glycosylation site of a protein, but may also be introduced on a different site on a protein.

In FIG. 4 several examples of a GlcNAc moiety according to Formula (1), wherein said GlcNAc moiety is a glycoprotein, are shown. FIG. 4 shows a glycoprotein according to Formula (11) wherein the core-GalNAc of the glycan is bonded via an O-glycosidic bond to the amino acid residue of the glycoprotein. FIG. 4 also shows a glycoprotein according to Formula (11) wherein the core-GlcNAc of the glycan is bonded via an N-glycosidic bond to the amino acid residue of the glycoprotein, wherein the core-GlcNAc is fucosylated (b is 1) and wherein the core-GlcNAc is non-fucosylated (b is 0).

In a preferred embodiment of the process according to the invention, the GlcNAc moiety according to Formula (1) is an antibody. In this embodiment, the GlcNAc in GlcNAc moiety (1) is the terminal GlcNAc of an antibody glycan, i.e. in this embodiment A in GlcNAc moiety (1) is an antibody. Preferably, the antibody is an antibody according to formula (10) or (11) as defined above, wherein Pr is Ab. In this embodiment, it is further preferred that y is 1, 2, 3, 4, 5, 6, 7 or 8. In this embodiment, it is further preferred that the antibody is according to Formula (10) as defined above. An antibody according to Formula (10) may be provided in several ways, for example by trimming of an antibody glycan with an endo-glycosidase, as described in EMBO J. 2001, 12, 3046 (incorporated by reference).

The antibody may be a whole antibody, but also an antibody fragment. When the antibody is a whole antibody, said antibody preferably comprises one or more, more preferably one, glycans on each heavy chain. Said antibody may also contain zero, one or more glycans on the light chain. Said whole antibody thus preferably comprises 2 or more, preferably 2, 4, 6 or 8 of said glycans, more preferably 2 or 4, and most preferably 2 glycans. In other words, when said antibody is a whole antibody, y is preferably 2, 4, 6 or 8, more preferably y is 2 or 4, and most preferably y is 2. When the antibody is an antibody fragment, it is preferred that y is 1, 2, 3 or 4, and more preferably y is 1 or 2.

In a particular preferred embodiment, when said glycoprotein is an antibody, y is 1, 2 or 4.

In a preferred embodiment, said antibody is a monoclonal antibody (mAb). Preferably, said antibody is selected from the group consisting of IgA, IgD, IgE, IgG and IgM antibodies. More preferably, said antibody is an IgG antibody, and most preferably said antibody is an IgG1 antibody. In a further preferred embodiment, the glycan in the antibody is attached to the conserved N-glycosylation site in the Fc-fragment at asparagine in the region 290-305, typically N297.

In the process according to the invention, when GlcNAc moiety (1) is a glycoprotein according to Formula (10) or (11), it is preferred that the N-acetylgalactosamine-(hetero)aryl moiety is according to Formula (3a) or (3b) as defined above.

As was described above, in this embodiment of the process according to the invention it is further preferred that Nuc is UDP, i.e. preferably Nuc-GalNAryl is UDP-GalNAryl in this embodiment of the process.

In this embodiment of the process according to the invention it is further preferred that the (hetero)aryl group T in Nuc-GalNAryl comprises a functional group Z. In a further preferred embodiment, Z is a 1,3-dipolar functional group. 1,3-Dipolar functional groups are described in more detail above. In this embodiment it is further preferred that the 1,3-dipolar group is selected from the group consisting of an azide group, a nitrone group, a nitrile oxide group and a diazo group. More preferably, the 1,3-dipole functional group is selected from the group consisting of an azide group, a nitrone group and a nitrile oxide group. Most preferably, the 1,3-dipolar functional group is an azide group.

In this embodiment it is further preferred that the (hetero)aryl group comprises one or more electron-withdrawing substituents. Preferably the one or more electron-withdrawing subsituent is present on a Cβ carbon atom (i.e. a carbon atom adjacent to the Ca carbon atom that Z is bonded to). It is further preferred that the electron-withdrawing substituent is selected from the group consisting of F, Cl, Br, I, NO₂, CN, CO₂R, C(O)NHR and C(O)NR₂.

In a particularly preferred embodiment of the process according to the invention wherein the GalNAc moiety is a glycoprotein, the Nuc-GalNAryl is according to Formula (23), (23b) or preferred embodiments of (23b) as described above.

Preferably y is 1 to 12, more preferably y is 1, 2, 3, 4, 5, 6, 7 or 8, and even more preferably y is 1, 2, 3 or 4. Most preferably y is 1 or 2. In another preferred embodiment, y is 2, 4, 6 or 8, preferably 2 or 4, most preferably 2. This embodiment is particularly preferred when the glycoprotein is an antibody (Ab), i.e. when Pr is Ab, as described in more detail below. In one embodiment of the process according to the invention, the glycoprotein according to Formula (10) or (11) is an antibody. Glycoproteins and antibodies are described in more detail below.

A glycoprotein comprising a glycan comprising terminal GlcNAc-moiety at the non-reducing end, i.e. a terminal non-reducing GlcNAc protein, may be provided in several ways, for example by (a) trimming of N-glycoprotein with an endo-glycosidase as described in EMBO J. 2001, 12, 3046 (incorporated by reference) or (b) expression of hybrid N-glycoprotein in the presence of swainsonine as for example described by Satoh et al. in Glycobiology 2006, 17, 104-118, incorporated by reference (followed by sialidase/galactosidase treatment).

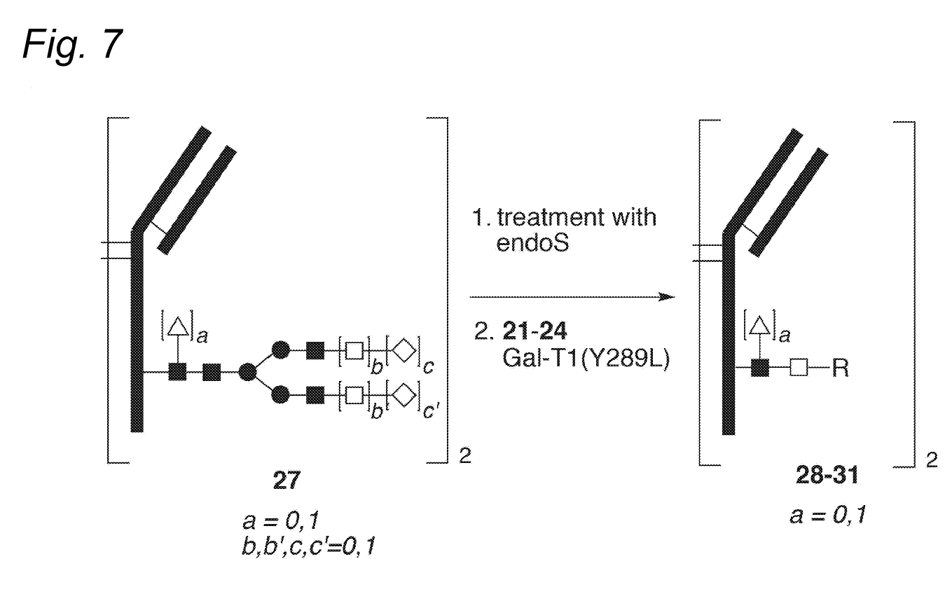

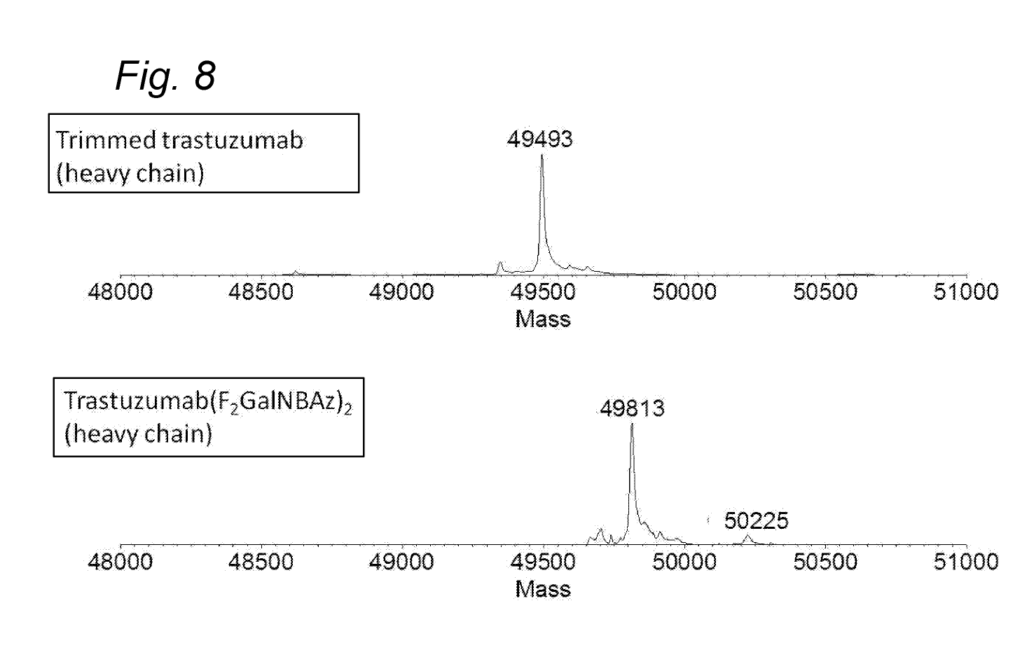

This preferred embodiment of the process according to the invention is shown in FIG. 7.

When GalNAryl of Nuc-GalNAryl is attached to GlcNAc of GlcNAc moiety (10), a glycoprotein according to Formula (8) is obtained, and when GalNAryl of Nuc-GalNAryl is attached to GlcNAc of GlcNAc moiety (11), a glycoprotein according to Formula (9) is obtained:

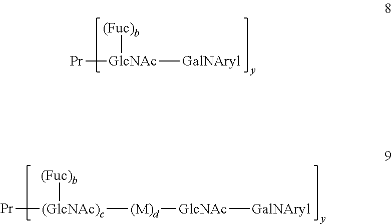

wherein:
GalNAryl is according to Formula (6) as defined above;
y is 1-20;
b is 0 or 1;
c is 0 or 1;
d is 0 or 1;
Pr is a glycoprotein; and
M is a monosaccharide, or a linear or branched oligosaccharide comprising 2 to 20 saccharide moieties.

The glycoprotein according to Formula (8) and (9) is described in more detail below.

As described in more detail above, the present invention relates to a process for attaching an N-acetylglucosamine moiety according to Formula (1) to an N-acetylglucosamine moiety according to Formula (2), by the action of a mutant galactosyltransferase.

The present invention further relates to a product obtainable by the process according to the invention.

The process according to the invention, GlcNAc moiety (1) and GalNAryl moiety (2) are described in more detail above.

The invention also relates to a compound according to Formula (5):

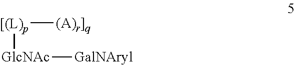

wherein:
L, A, p, r and q are as defined above for (1); and
GalNAryl is according to Formula (6):

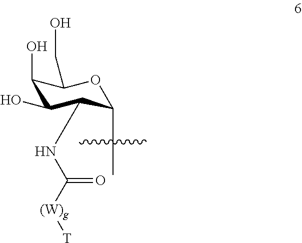

wherein:
W, T and g are as defined above for (2); and
T is optionally substituted.

W, g, T and optional substituents on T are described in more detail above and below.

In a preferred embodiment, the invention relates to a compound according to Formula (5) as described above, wherein GalNAryl is according to Formula (7):

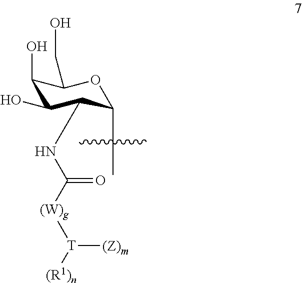

wherein:
T, W and g are as defined above for (2); and
R¹, Z, m and n are as defined above for (3b).

T is a (hetero)aryl group, i.e. an aryl group or a heteroaryl group. T may be any aryl group or any heteroaryl group. Preferred (hetero)aryl groups described in more detail above.

In a preferred embodiment of the product according to the invention, T is selected from the group consisting of phenyl groups, naphthyl groups, anthracyl groups, pyrrolyl groups, pyrrolium groups, furanyl groups, thiophenyl groups (i.e. thiofuranyl groups), pyrazolyl groups, imidazolyl groups, isoxazolyl groups, oxazolyl groups, oxazoliumgroups, isothiazolyl groups, thiazolyl groups, 1,2,3-triazolyl groups, 1,3,4-triazolyl groups, diazolyl groups, 1-oxa-2,3-diazolyl groups, 1-oxa-2,4-diazolyl groups, 1-oxa-2,5-diazolyl groups, 1-oxa-3,4-diazolyl groups, 1-thia-2,3-diazolyl groups, 1-thia-2,4-diazolyl groups, 1-thia-2,5-diazolyl groups, 1-thia-3,4-diazolyl groups, tetrazolyl groups, pyridinyl groups, pyridazinyl groups, pyrimidinyl groups, pyrazinyl groups, pyradizinyl groups, pyridiniumyl groups, pyrimidinium groups, benzofuranyl groups, benzothiophenyl groups, benzimidazolyl groups, indazolyl groups, benzotriazolyl groups, pyrrolo[2,3-b]pyridinyl groups, pyrrolo[2,3-c]pyridinyl groups, pyrrolo[3,2-c]pyridinyl groups, pyrrolo[3,2-b]pyridinyl groups, imidazo[4,5-b]pyridinyl groups, imidazo[4,5-c]pyridinyl groups, pyrazolo[4,3-d]pyridinyl groups, pyrazolo[4,3-c]pyridinyl groups, pyrazolo[3,4-c]pyridinyl groups, pyrazolo[3,4-b]pyridinyl groups, isoindolyl groups, indazolyl groups, purinyl groups, indolininyl groups, imidazo[1,2-a]pyridinyl groups, imidazo[1,5-a]pyridinyl groups, pyrazolo[1,5-a]pyridinyl groups, pyrrolo[1,2-b]pyridazinyl groups, imidazo[1,2-c]pyrimidinyl groups, quinolinyl groups, isoquinolinyl groups, cinnolinyl groups, quinazolinyl groups, quinoxalinyl groups, phthalazinyl groups, 1,6-naphthyridinyl groups, 1,7-naphthyridinyl groups, 1,8-naphthyridinyl groups, 1,5-naphthyridinyl groups, 2,6-naphthyridinyl groups, 2,7-naphthyridinyl groups, pyrido[3,2-d]pyrimidinyl groups, pyrido[4,3-d]pyrimidinyl groups, pyrido[3,4-d]pyrimidinyl groups, pyrido[2,3-d]pyrimidinyl groups, pyrido[2,3-b]pyrazinyl groups, pyrido[3,4-b]pyrazinyl groups, pyrimido[5,4-d]pyrimidinyl groups, pyrazino[2,3-b]pyrazinyl groups and pyrimido[4,5-d]pyrimidinyl groups.

In a further preferred embodiment, T is selected from the group consisting of phenyl groups, pyridinyl groups, pyridiniumyl groups, pyrimidinyl groups, pyrimidinium groups, pyrazinyl groups, pyradizinyl groups, pyrrolyl groups, pyrrolium groups, furanyl groups, thiophenyl groups (i.e. thiofuranyl groups), diazolyl groups, quinolinyl groups, imidazolyl groups, oxazolyl groups and oxazolium groups.

Optionally, (hetero)aryl group T is substituted with one or more substituents R¹. In a preferred embodiment, n is 0. In another preferred embodiment, n is 1, 2, 3 or 4, more preferably n is 1 or 2, and most preferably n is 1.

If present, R¹ is independently selected from the group consisting of C₁-C₁₂ alkyl groups, C₂-C₁₂ (hetero)aryl groups, C₃-C₁₂ alkyl(hetero)aryl groups, C₃-C₁₂ (hetero)arylalkyl groups, C₂-C₁₂ alkenyl groups, C₂-C₁₂ alkynyl groups, C₃-C₁₂ cycloalkyl groups, C₅-C₁₂ cycloalkenyl groups, C₈-C₁₂ cycloalkynyl groups, C₁-C₁₂ alkoxy groups, C₂-C₁₂ alkenyloxy groups, C₂-C₁₂ (hetero)aryloxy groups, C₃-C₁₂ alkyl(hetero)aryl groups, C₃-C₁₂ (hetero)arylalkyl groups, C₂-C₁₂ alkynyloxy groups and C₃-C₁₂ cycloalkyloxy groups, wherein the alkyl groups, (hetero)aryl groups, alkyl(hetero)aryl groups, (hetero)arylalkyl groups, alkenyl groups, alkynyl groups, cycloalkyl groups, alkoxy groups, alkenyloxy groups, (hetero)aryloxy groups, alkynyloxy groups and cycloalkyloxy groups are optionally substituted, the alkyl groups, the alkoxy groups, the cycloalkyl groups and the cycloalkoxy groups being optionally interrupted by one of more hetero-atoms selected from the group consisting of O, N and S. More preferably, R¹ is independently selected from the group consisting of C₁-C₁₂ alkyl groups, C₃-C₁₂ cycloalkyl groups, C₂-C₁₂ (hetero)aryl groups, C₃-C₁₂ alkyl(hetero)aryl groups and C₃-C₁₂ (hetero)arylalkyl groups, wherein the alkyl groups, cycloalkyl groups, (hetero)aryl groups, alkyl(hetero)aryl groups and (hetero)arylalkyl groups are optionally substituted, wherein the alkyl groups, cycloalkyl groups, alkyl(hetero)aryl groups and (hetero)arylalkyl groups are optionally interrupted by one or more heteroatoms selected from the group consisting of O, S and N.

Even more preferably, R¹ is independently selected from the group consisting of C₁-C₆ alkyl groups, C₃-C₆ cycloalkyl groups, C₂-C₆ (hetero)aryl groups, C₃-C₆ alkyl(hetero)aryl groups and C₃-C₆ (hetero)arylalkyl groups, wherein the alkyl groups, cycloalkyl groups, (hetero)aryl groups, alkyl(hetero)aryl groups and (hetero)arylalkyl groups are optionally substituted, wherein the alkyl groups, cycloalkyl groups, alkyl(hetero)aryl groups and (hetero)arylalkyl groups are optionally interrupted by one or more heteroatoms selected from the group consisting of O, S and N. Even more preferably, R¹ is independently selected from the group consisting of C₁-C₆ alkyl groups, yet even more preferably R¹ is methyl, ethyl, n-propyl, i-propyl, n-butyl or t-butyl. Most preferably R¹ is methyl, ethyl or i-propyl.

(Hetero)aryl group T is linked to the C(O) group of the galactosamine moiety, either directly (g is 0) or via W (g is 1). When present, W is preferably selected from the group consisting of C₁-C₁₂ alkylene groups, C₂-C₁₂ alkenylene groups, C₃-C₁₂ cycloalkylene groups, C₂-C₁₂ (hetero)arylene groups, C₃-C₁₂ alkyl(hetero)arylene groups and C₃-C₁₂ (hetero)arylalkylene groups, wherein the alkylene groups, alkenylene groups, cycloalkylene groups, (hetero)arylene groups, alkyl (hetero)aryl ene groups and (hetero)arylalkylene groups are optionally substituted, and wherein the alkylene groups, alkenylene groups, cycloalkylene groups, (hetero)arylene groups, alkyl(hetero)arylene groups and (hetero)arylalkylene groups are optionally interrupted by one or more heteroatoms selected from the group consisting of O, S and N.

More preferably, W is selected from the group consisting of C₁-C₆ alkylene groups, C₂-C₆ alkenylene groups, C₃-C₆ cycloalkylene groups, C₂-C₈ (hetero)arylene groups, C₃-C₆ alkyl(hetero)arylene groups and C₃-C₆ (hetero)arylalkylene groups, wherein the alkylene groups, alkenylene groups, cycloalkylene groups, (hetero)arylene groups, alkyl(hetero)arylene groups and (hetero)arylalkylene groups are optionally substituted, and wherein the alkylene groups, alkenylene groups, cycloalkylene groups, (hetero)arylene groups, alkyl(hetero)arylene groups and (hetero)arylalkylene groups are optionally interrupted by one or more heteroatoms selected from the group consisting of O, S and N.

More preferably, W is selected from the group consisting of C₁-C₆ alkylene groups and C₂-C₆ (hetero)arylene groups.

Most preferably, W is selected from the group consisting of methylene, ethylene, propylene, butylene (preferably n-butylene), pentylene (preferably n-pentylene) and hexylene (preferably n-hexylene).

In a preferred embodiment, g is 1. In another preferred embodiment, g is 0.

When m is 2 or more, i.e. when more than 1 functional group Z is present on the (hetero)aryl group T, the functional groups Z are independently selected. In other words, (hetero)aryl group T may be substituted with more than one type of functional group. For example, the (hetero)aryl group may be substituted with a 1,3-dipole functional group, and one or more halogens.

In a preferred embodiment of the product according to the invention, Z is independently selected from the group consisting of a 1,3-dipole functional group, halogen (F, Cl, Br, I), R³, —CH═C(R³)₂, —C≡CR³, —[C(R³)₂C(R³)₂O]_{q}—R³ wherein q is in the range of 1 to 200, —CN, —NC, NO₂, —NCX, —XCN, —XR³, —N(R³)₂, —⁺N(R³)₃, —C(X)N(R³)₂, —C(R³)₂XR³, —C(X)R³, —C(X)XR³, —S(O)R³, —S(O)₂R³, —S(O)OR³, —S(O)₂OR³, —S(O)N(R³)₂, —S(O)₂N(R³)₂, —OS(O)R³, —OS(O)₂R³, —OS(O)OR³, —OS(O)₂OR³, —P(O)(R³)(OR³), —P(O)(OR³)₂, —OP(O)(OR³)₂, —Si(R³)₃, —XC(X)R³, —XC(X)XR³, —XC(X)N(R³)₂, —N(R³)C(X)R³, —N(R³)C(X)XR³ and —N(R³)C(X)N(R³)₂, wherein X is oxygen or sulphur and wherein R³ is independently selected from the group consisting of hydrogen, halogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups optionally substituted and optionally interrupted by one or more heteroatoms selected from O and N. In this embodiment, it is further preferred that X is O.

Preferably R³ is independently selected from the group consisting of hydrogen, halogen and C₁-C₆ alkyl groups, more preferably from the group consisting of hydrogen, halogen and C₁-C₄ alkyl groups. Most preferably, R³ is independently selected from the group consisting of hydrogen, methyl, ethyl, propyl, i-propyl, butyl and t-butyl. X is preferably oxygen.

More preferably, Z is independently selected from the group consisting of a 1,3-dipole functional group, halogen (F, Cl, Br, I), —CN, —NCX, —XCN, —XR³, —N(R³)₂, —⁺N(R³)₃, —C(X)N(R³)₂, —C(R³)₂XR³, —C(X)R³, —C(X)XR³, —XC(X)R³, —XC(X)XR³, —XC(X)N(R³)₂, —N(R³)C(X)R³, —N(R³)C(X)XR³ and —N(R³)C(X)N(R³)₂, wherein X and R³, and preferred embodiments of X and R³, are as defined above.

Most preferably, Z is selected from the group consisting of a 1,3-dipole functional group, halogen (F, Cl, Br, I), —OR³, —SR³, —N(R³)₂, —⁺N(R³)₃, —C(O)N(R³)₂, —C(O)OR³, —OC(O)R³, —OC(O)OR³, —OC(O)N(R³)₂, —N(R³)C(O)R³, —N(R³)C(O)OR³ and —N(R³)C(O)N(R³)₂, wherein X and R³, and preferred embodiments of X and R³, are as defined above.

When Z is halogen, i.e. Z is F, Cl, Br or I, it is preferred that Z is F, Cl or Br, and preferably F or Cl, and most preferably F.

Optionally, functional group Z is masked or protected.

Most preferably, Z is selected from the group consisting of a 1,3-dipole functional group, halogen (F, Cl, Br, I), —OR³, —SR³, —N(R³)₂, —⁺N(R³)₃, —C(O)N(R³)₂, —C(O)OR³, —OC(O)R³, —OC(O)OR³, —OC(O)N(R³)₂, —N(R³)C(O)R³, —N(R³)C(O)OR³ and —N(R³)C(O)N(R³)₂, wherein X and R³, and preferred embodiments of X and R³, are as defined above.

When Z is halogen, i.e. Z is F, Cl, Br or I, it is preferred that Z is F, Cl or Br, and preferably F or Cl, and most preferably F.

It is further preferred that Z is independently selected from the group consisting of a 1,3-dipole functional group, F, Cl, Br, I, —CN, —OR³, —SR³ and —N(R³)₂, wherein R³ is as defined above. More preferably Z is independently selected from the group consisting of a 1,3-dipole functional group, —F, —Cl, —Br, —CN, —OH and —SH, even more preferably from the group consisting of a 1,3-dipole functional group, —F, —Cl, —Br, —OH and —SH. Most preferably, Z is independently selected from the group consisting of an azide group, a nitrone group, a nitrile oxide group, a diazo group, —F, —Cl, —OH and —SH.

In a preferred embodiment of (3b) m is 1, 2, 3, 4 of 5. When m is 2 or more, the (hetero)aryl group may be substituted with 2 or more different functional groups Z. For example, the (hetero)arylgroup may be substituted with a 1,3-dipole group and with one or more halogens. In a further preferred embodiment, the (hetero)aryl group in (3b) comprises a 1,3-dipole group, and optionally 2 or 4 halogen atoms, preferably F or Cl atoms. In a particularly preferred embodiment, the (hetero)aryl group T comprises an azide group and two F-atoms, or an azide group and four F atoms.

In another preferred embodiment m is 0. In this embodiment it is further preferred that n is 0. In this embodiment, it is therefore preferred that the (hetero)aryl group T is unsubstituted.

In the product according to Formula (5), and preferred embodiments thereof, preferably n is 0 and g is 0. In a further preferred embodiment, n is 0, g is 0 and m is 0. In another further preferred embodiment, n is 0, g is 0 and m is 1, 2, 3 or 4. In another further preferred embodiment, n is 0, g is 0 and m is 2. In another further preferred embodiment, n is 0, g is 0 and m is 4.

As was described above, in a preferred embodiment of the process according to the invention, the N-acetylglucosamine moiety is a terminal GlcNAc moiety of a glycoprotein glycan. Therefore the invention further relates to a glycoprotein according to Formula (8) or (9):

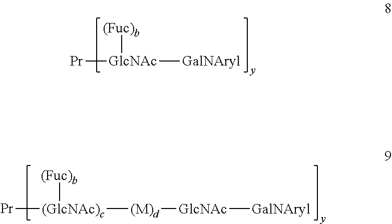

wherein:
GalNAryl is according to Formula (6) as defined above;
y is 1-20;
b is 0 or 1;
c is 0 or 1;
d is 0 or 1;
Pr is a glycoprotein; and
M is a monosaccharide, or a linear or branched oligosaccharide comprising 2 to 20 saccharide moieties.

In a preferred embodiment of glycoprotein (8) and (9), GalNAryl is according to Formula (7) as defined above. GalNAryl (6), GalNAryl (7) and preferred embodiments of (6) and (7) are described in more detail above. These preferred embodiments are also applicable to GalNAryl in the glycoprotein according to Formula (8) and (9).

A glycoprotein according to Formula (8) or (9) may comprise more than one glycan (y is 1-20), and may comprise a combination of N-linked, O-linked and C-linked glycans. Preferably, y is 1 to 12, more preferably y is 1, 2, 3, 4, 5, 6, 7 or 8, and even more preferably y is 1, 2, 3 or 4. Most preferably y is 1 or 2.

In yet another preferred embodiment, y is 2, 4, 6 or 8, preferably 2 or 4, most preferably 2. This embodiment is particularly preferred when the glycoprotein is an antibody (Ab), i.e. when Pr is Ab, as described in more detail below.

The glycan may be present at a native glycosylation site of the protein, but also on a different site on the protein.

In a further preferred embodiment, the glycoprotein is an antibody (Ab), i.e. Pr in (8) and (9) is Ab. In this embodiment, y is 1, 2, 3, 4, 5, 6, 7 or 8, preferably 1, 2, 3 or 4, most preferably 1 or 2. This embodiment is particularly preferred when the glycoprotein is an antibody (Ab). The antibody may be a whole antibody, but also an antibody fragment. When the antibody is a whole antibody, said antibody preferably comprises one or more, more preferably one, glycans on each heavy chain. Said whole antibody thus preferably comprises 2 or more, preferably 2, 4, 6 or 8 of said glycans, more preferably 2 or 4, and most preferably 2 glycans. In other words, when said antibody is a whole antibody, y is preferably 2, 4, 6 or 8, more preferably y is 2 or 4, and most preferably y is 2. When the antibody is an antibody fragment, it is preferred that y is 1, 2, 3 or 4, and more preferably y is 1 or 2.

In a particular preferred embodiment, when glycoprotein (8) or (9) is an antibody, y is 1, 2 or 4.

In a preferred embodiment, said antibody is a monoclonal antibody (mAb). Preferably, said antibody is selected from the group consisting of IgA, IgD, IgE, IgG and IgM antibodies. More preferably, said antibody is an IgG antibody, and most preferably said antibody is an IgG1 antibody. In a further preferred embodiment, the glycan in the antibody is attached to the conserved N-glycosylation site in the Fc-fragment at asparagine in the region 290-305, typically N297.

When the glycoprotein according to Formula (8) or (9) is an antibody, the antibody may be further used e.g. in the preparation of an Antibody-Drug Conjugate (ADC). For example when Z in GalNAryl is an azide group, the antibody (8) or (9) may be further reacted with a conjugate comprising a (hetero)cycloalkyne and a molecule of interest, e.g. a cytotoxin. Therefore, in a preferred embodiment, when the glycoprotein according to Formula (8) or (9) is an antibody, said antibody is used in the preparation of an Antibody-Drug Conjugate.

Preferred embodiments for Z are described above. In glycoprotein (8) and (9), it is further preferred that Z is independently selected from the group consisting of a 1,3-dipole functional group, F, Cl, Br, I, —CN, —OR³, —SR³ and —N(R³)₂, wherein R³ is as defined above. More preferably Z is independently selected from the group consisting of a 1,3-dipole functional group, —F, —Cl, —Br, —CN, —OH and —SH, even more preferably from the group consisting of a 1,3-dipole functional group, —F, —Cl, —Br, —OH and —SH. Most preferably, Z is independently selected from the group consisting of an azide group, a nitrone group, a nitrile oxide group, a diazo group, —F, —Cl, —OH and —SH.

In a preferred embodiment m is 0. In this embodiment it is further preferred that n is 0.

In a preferred embodiment m is 1, 2, 3, 4 of 5. When m is 2 or more, the (hetero)aryl group may be substituted with 2 or more different functional groups Z. For example, the (hetero)arylgroup may be substituted with a 1,3-dipole group and with one or more halogens. In a preferred embodiment, the (hetero)aryl group in glycoprotein (8) and (9) comprises a 1,3-dipole group and 2 or 4 halogen atoms, preferably F or Cl atoms. In a particularly preferred embodiment, the (hetero)aryl group T comprises an azide group and two F-atoms, or an azide group and two Cl-atoms. It is further preferred that the azide group is on the para position relative to (W)_{g}, and that both F or Cl atoms are on the meta position relative to (W)_{g}, i.e. on the ortho position relative to the azide group. In these embodiments it is further preferred that n is 0.

In another particularly preferred embodiment, the (hetero)aryl group T comprises an azide group and four F-atoms, or an azide group and four Cl-atoms. Preferably the azide group is on the para position relative to (W)_{g}.

In the glycoprotein according to Formula (8) or (9) the GalNAryl is bonded to the GlcNAc via an O-glycosidic linkage. The GalNAryl of Nuc-GalNAryl may for example be bonded via C1 to C4 of the GlcNAc via a β(1,4)-glycosidic bond, or to C3 of said GlcNAc via an α(1,3)-glycosidic bond. As was described above, the type of glycosidic bond that is present in (5) depends on the type of enzyme that catalysed its formation.

In a particularly preferred embodiment of the glycoprotein according to Formula (8) or (9), GalNAryl is according to Formula (23f), (21f) or (21g):

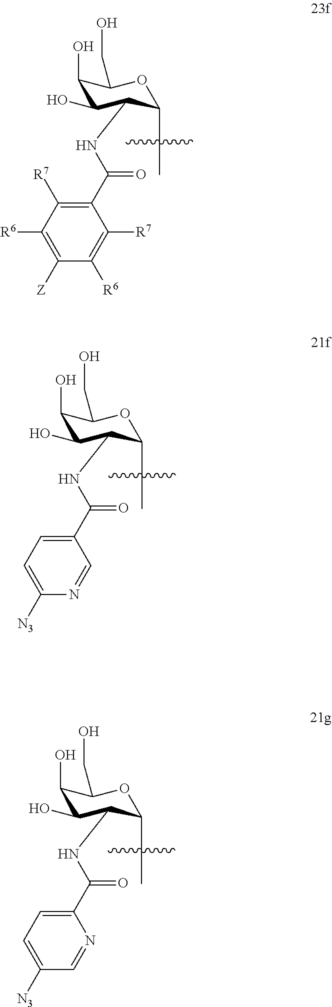

wherein:
Z is a functional group;
R⁶ is independently selected from the group consisting of hydrogen, F, Cl, Br and I; and
R⁷ is independently selected from the group consisting of hydrogen, F, Cl, Br and I.

When GalNAryl is according to Formula (23f), it is further preferred that R⁶ is independently selected from the group consisting of hydrogen, F and Cl, and that R⁷ is independently selected from the group consisting of hydrogen, F and Cl. More preferably R⁶ and R⁷ are independently hydrogen or F. In a further preferred embodiment R⁷ is hydrogen and R⁶ is F. In another further preferred embodiment, R⁶ and R⁷ are F. In these embodiments it is further preferred that Z is an azide group.

In the process according to the invention a GlcNAc moiety according to Formula (1) is attached to a GalNAryl moiety according to Formula (2). The invention also relates to a compound according to Formula (3b):

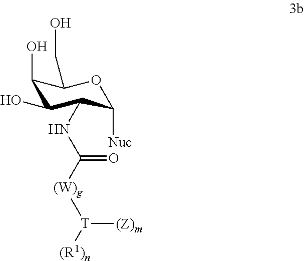

wherein:
Nuc is a nucleotide;
W, g and T are as defined above for GalNAryl (6); and
Z, R¹, m and n are as defined above for GalNAryl (7).

GalNAryl (6), GalNAryl (7) and preferred embodiments of (6) and (7) are described in more detail above, and are also applicable the compound according to Formula (3b).

The term “Nuc” herein refers to a nucleotide. Nucleotides are well known in the art, and the term “nucleotide” is herein used in its normal scientific meaning. In the process according to the invention, Nuc is preferably selected from the group consisting of a nucleoside monophosphate and a nucleoside diphosphate, more preferably from the group consisting of uridine diphosphate (UDP), guanosine diphosphate (GDP), thymidine diphosphate (TDP), cytidine diphosphate (CDP) and cytidine monophosphate (CMP), more preferably from the group consisting of uridine diphosphate (UDP), guanosine diphosphate (GDP), cytidine diphosphate and (CDP). Most preferably, Nuc is UDP.

W and preferred embodiments thereof are described in more detail above. In a preferred embodiment g is 1, and W is preferably selected from the group consisting of methylene, ethylene, n-propylene, i-propylene, butylene (preferably n-butylene), pentylene (preferably n-pentylene) and hexylene (preferably n-hexylene).

In another preferred embodiment, g is 0.

In a further preferred embodiment, T is selected from the group consisting of phenyl groups, pyridinyl groups, pyridiniumyl groups, pyrimidinyl groups, pyrimidinium groups, pyrazinyl groups, pyradizinyl groups, pyrrolyl groups, pyrrolium groups, furanyl groups, thiophenyl groups (i.e. thiofuranyl groups), diazolyl groups, quinolinyl groups, imidazolyl groups, oxazolyl groups and oxazolium groups.

When m is 2 or more, i.e. when more than 1 functional group Z is present on the (hetero)aryl group T, the functional groups Z are independently selected. In other words, (hetero)aryl group T may be substituted with more than one type of functional group. For example, the (hetero)aryl group may be substituted with a 1,3-dipole functional group, and one or more halogens.

Most preferably, Z is selected from the group consisting of a 1,3-dipole functional group, halogen (F, Cl, Br, I), —OR³, —SR³, —N(R³)₂, —⁺N(R³)₃, —C(O)N(R³)₂, —C(O)OR³, —OC(O)R³, —OC(O)OR³, —OC(O)N(R³)₂, —N(R³)C(O)R³, —N(R³)C(O)OR³ and —N(R³)C(O)N(R³)₂, wherein X and R³, and preferred embodiments of X and R³, are as defined above.

When Z is halogen, i.e. Z is F, Cl, Br or I, it is preferred that Z is F, Cl or Br, and preferably F or Cl, and most preferably F.

It is further preferred that Z is independently selected from the group consisting of a 1,3-dipole functional group, F, Cl, Br, I, —CN, —OR³, —SR³ and —N(R³)₂, wherein R³ is as defined above. More preferably Z is independently selected from the group consisting of a 1,3-dipole functional group, —F, —Cl, —Br, —CN, —OH and —SH, even more preferably from the group consisting of a 1,3-dipole functional group, —F, —Cl, —Br, —OH and —SH. Most preferably, Z is independently selected from the group consisting of an azide group, a nitrone group, a nitrile oxide group, a diazo group, —F, —Cl, —OH and —SH.

In a preferred embodiment of (3b) m is 1, 2, 3, 4 or 5. When m is 2 or more, the (hetero)aryl group may be substituted with 2 or more different functional groups Z. For example, the (hetero)arylgroup may be substituted with a 1,3-dipole group and with one or more halogens. In a further preferred embodiment, the (hetero)aryl group in (3b) comprises a 1,3-dipole group, and optionally 2 or 4 halogen atoms, preferably F or Cl atoms. In a particularly preferred embodiment, the (hetero)aryl group T comprises an azide group and two F-atoms, or an azide group and four F atoms.

In another preferred embodiment m is 0. In this embodiment it is further preferred that n is 0. In this embodiment, it is therefore preferred that the (hetero)aryl group T is unsubstituted.

When in the compound according to Formula (3b) the (hetero)aryl group T is an, optionally substituted, phenyl group, it is preferred that m and n are not both 0. The invention therefore also relates to a compound according to Formula (3b) as defined above, with the proviso that when T is a phenyl group, m and n are not both 0.

The invention also relates to a compound according to Formula (23b) or (23):

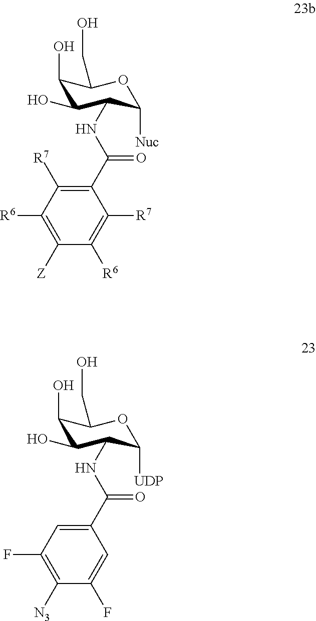

wherein:
Nuc is a nucleotide;
Z is a functional group;
R⁶ is independently selected from the group consisting of hydrogen, F, Cl, Br and I; and
R⁷ is independently selected from the group consisting of hydrogen, F, Cl, Br and I.

Nuc is preferably selected from the group consisting of a nucleoside monophosphate and a nucleoside diphosphate, more preferably from the group consisting of uridine diphosphate (UDP), guanosine diphosphate (GDP), thymidine diphosphate (TDP), cytidine diphosphate (CDP) and cytidine monophosphate (CMP), more preferably from the group consisting of uridine diphosphate (UDP), guanosine diphosphate (GDP), cytidine diphosphate and (CDP). Most preferably, Nuc is UDP.

Z is a functional groups. Preferred embodiments of Z are as described above for GalNAryl (7). It is further preferred that Z is independently selected from the group consisting of a 1,3-dipole functional group, F, Cl, Br, I, —CN, —OR³, —SR³ and —N(R³)₂, wherein R³ is as defined above. More preferably Z is independently selected from the group consisting of a 1,3-dipole functional group, —F, —Cl, —Br, —CN, —OH and —SH, even more preferably from the group consisting of a 1,3-dipole functional group, —F, —Cl, —Br, —OH and —SH. Even more preferably, Z is selected from the group consisting of an azide group, a nitrone group, a nitrile oxide group, a diazo group, —F, —Cl, —OH and —SH. Most preferably, Z is a 1,3-dipole functional group, most preferably an azide group.

In a preferred embodiment, R⁶ and R⁷ are hydrogen. In another preferred embodiment, R⁶ is F and R⁷ is hydrogen. In another preferred embodiment, R⁶ is Cl and R⁷ is hydrogen. In another preferred embodiment, R⁶ is F and R⁷ is F. In another preferred embodiment, R⁶ is Cl and R⁷ is Cl. In these embodiments it is further preferred that Z is an azide group.

The invention further relates to a compound according to Formula (23), (23c), (23d) or (23e):

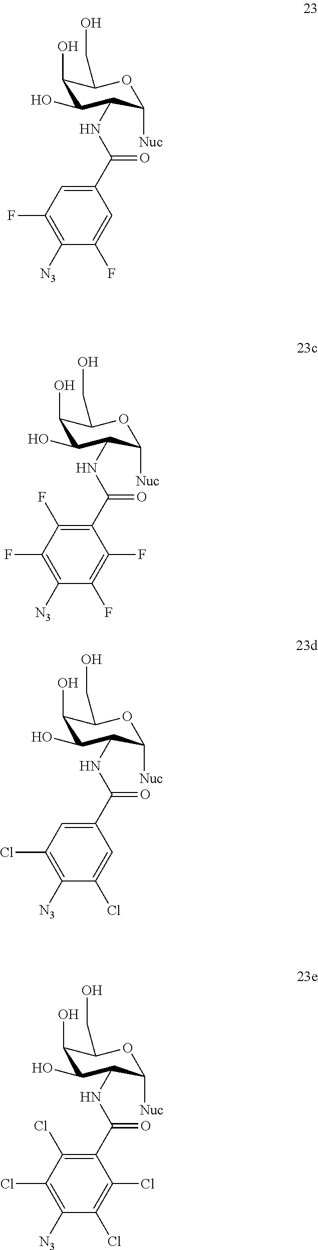

wherein Nuc is a nucleotide, as defined above.

Also in (23), (23c), (23d) or (23e), most preferably, Nuc is UDP.

The process and the products according to the invention have several advantages. For example, one field of application involves medicinal chemistry where the selective introduction of an aryl-substituted GalNAc onto a GlcNAc-containing medicinal product may impart specific binding interactions of the medicinal product with a biological target, thereby enhancing affinity and/or selectivity. In addition, carbohydrate microarrays may be constructed containing aryl-substituted GalNAc-moieties, which enables further diversification of the microarray and include enhanced selectivity. Upon enzymatic introduction onto a terminal GlcNAc moiety of a glycoprotein, new properties can be imparted upon this protein by means of the aromatic moiety such as aromatic stacking or particular absorbance properties. In a secondary mode, the aryl moiety on the modified glycoprotein, depending on the particular aryl group, may serve as an anchor point for subsequent regioselective chemical modification, such as for example electrophilic aromatic substitution, transition-metal catalyzed coupling, ring-closing metathesis. One particular example of the latter is that a very large advantage of the glycoprotein according to the invention is that the ensuing reaction with a cycloalkyne may have a significantly increased reaction rate depending on the particular aryl group substitution.

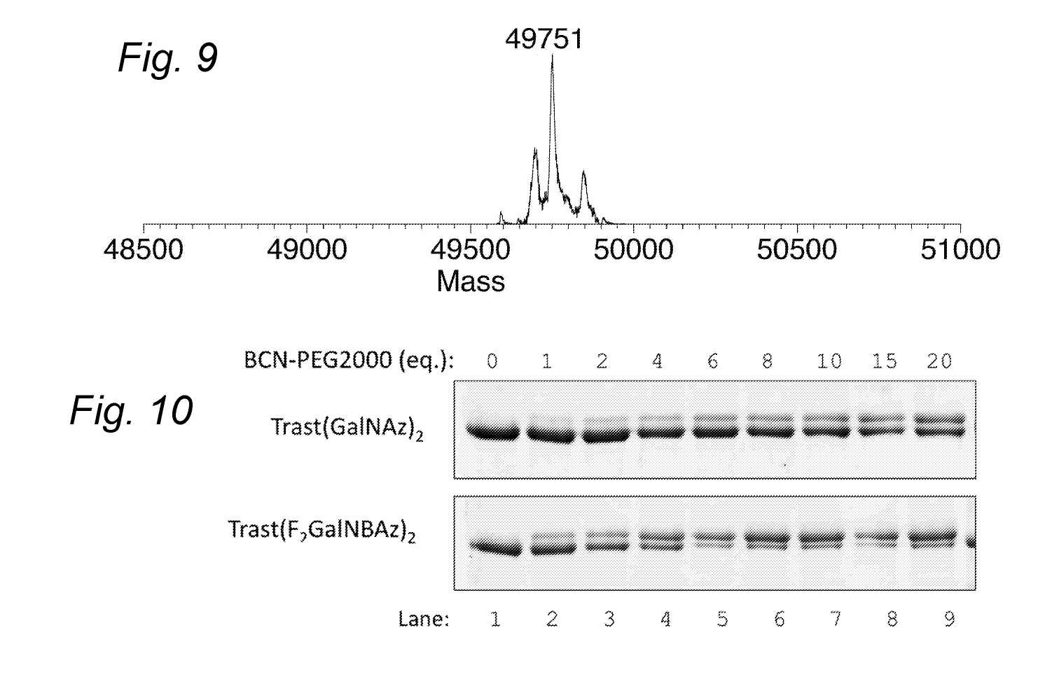

This may be seen in FIG. 10. FIG. 10 shows the heavy chain of trastuzumab-(GalNAz)₂ (top panel) and trastuzumab-(F₂-GalNBAz)₂ (lower panel) before conjugation to BCN-PEG₂₀₀₀ (lower band) and after conjugation to BCN-PEG₂₀₀₀ (upper band). Trast-(GalNAz)₂ shows less than 50% conversion when incubated with 20 equivalents BCN-PEG₂₀₀₀ (upper panel, lane 9) while trast-(F₂-GalNBAz)₂ shows >50% conversion when incubated with only 4 equivalents BCN-PEG₂₀₀₀ (lower panel, lane 4).

### EXAMPLES

### Example 1

### Synthesis of 2-azidogalactose 1-phosphate Derivative (17)

Compound 17 was prepared from D-galactosamine according to the procedure described for D-glucosamine in Linhardt et al., J. Org. Chem. 2012, 77, 1449-1456.

¹H-NMR (300 MHz, CD₃OD): δ 5.69 (dd, J=7.2, 3.3 Hz, 1H), 5.43-5.42 (m, 1H), 5.35 (dd, J=11.1, 3.3 Hz, 1H), 4.53 (t, J=7.2 Hz, 1H), 4.21-4.13 (m, 1H), 4.07-4.00 (m, 1H), 3.82 (dt, J=10.8, 2.7 Hz, 1H), 2.12 (s, 3H), 2.00 (s, 3H), 1.99 (s, 3H).

LRMS (ESI-) calcd for C₁₂H₁₇N₃O₁₁P (M−H⁺) 410.06, found 410.00.

### Example 2

### Synthesis of 2-azidogalactose UDP Derivative (18)

Compound 17 was attached to UMP according to Baisch et al. Bioorg. Med. Chem., 1997, 5, 383-391.

Thus, a solution of D-uridine-5′-monophosphate disodium salt (1.49 g, 4.05 mmol) in H₂O (15 mL) was treated with DOWEX 50W×8 (H⁺ form) for 30 minutes and filtered. The filtrate was stirred vigorously at room temperature while tributylamine (0.966 mL, 4.05 mmol) was added dropwise. After 30 minutes of further stirring, the reaction mixture was lyophilized and further dried over P₂O₅ under vacuum for 5 h.

The resulting tributylammonium uridine-5′-monophosphate was dissolved in dry DMF (25 mL) in an argon atmosphere. Carbonyldiimidazole (1.38 g, 8.51 mmol) was added and the reaction mixture was stirred at r.t. for 30 min. Next, dry MeOH (180 μL) was added and stirred for 15 min to remove the excess carbonyldiimidazole. The leftover MeOH was removed under high vacuum for 15 min. Subsequently, compound 26 (2.0 g, 4.86 mmol) was dissolved in dry DMF (25 mL) and added dropwise to the reaction mixture. The reaction was allowed to stir at rt for 2 d before concentration in vacuo. The consumption of the imidazole-UMP intermediate was monitored by MS. Flash chromatography (7:2:1-5:2:1 EtOAc:MeOH:H₂O) afforded product 18 (1.08 g, 1.51 mmol, 37%).

¹H-NMR (300 MHz, D₂O): δ 7.96 (d, J=8.0 Hz, 1H), 5.98-5.94 (m, 2H), 5.81-5.79 (m, 1H), 5.70 (dd, J=7.1, 3.3 Hz, 1H), 5.49 (dd, J=15.2, 2.6 Hz, 1H), 5.30 (ddd, J=18.5, 11.0, 3.2 Hz, 2H), 4.57 (q, J=6.0 Hz, 2H), 4.35-4.16 (m, 9H), 4.07-3.95 (m, 2H), 2.17 (s, 3H), 2.08 (s, 3H), 2.07 (s, 3H).

LRMS (ESI-) calcd for C₂₁H₂₉N₅O₁₉P₂ (M−H⁺) 716.09, found 716.3.

### Example 3

### Synthesis of Deacetylated 2-azidogalactose UDP Derivative (19)

Deacetylation was performed according to Kiso et al., Glycoconj. J., 2006, 23, 565.

Thus, compound 18 (222 mg, 0.309 mmol) was dissolved in H₂O (2.5 mL) and triethylamine (2.5 mL) and MeOH (6 mL) were added. The reaction mixture was stirred for 3 h and then concentrated in vacuo to afford crude UDP-2-azido-2-deoxy-D-galactose (19). ¹H-NMR (300 MHz, D₂O): δ 7.99 (d, J=8.2 Hz, 1H), 6.02-5.98 (m, 2H), 5.73 (dd, J=7.4, 3.4 Hz, 1H), 4.42-4.37 (m, 2H), 4.30-4.18 (m, 4H), 4.14-4.04 (m, 2H), 3.80-3.70 (m, 2H), 3.65-3.58 (m, 1H).

LRMS (ESI⁻) calcd for C₁₅H₂₃N₅O₁₆P₂ (M−H⁺) 590.05, found 590.2.

### Example 4

### Synthesis of UDP-Galactosamine (20)

To a solution of compound 19 in H₂O:MeOH 1:1 (4mL) was added Lindlar's catalyst (50 mg). The reaction was stirred under a hydrogen atmosphere for 5 h and filtered over celite. The filter was rinsed with H₂O (10 ml) and the filtrate was concentrated in vacuo to afford the UDP-D-galactosamine (UDP-GalNH₂, 20) (169 mg, 0.286 mmol, 92% yield over two steps). ¹H-NMR (300 MHz, D₂O): δ 7.93 (d, J=8.1 Hz, 1H), 5.99-5.90 (m, 2H), 5.76-5.69 (m, 1H), 4.39-4.34 (m, 2H), 4.31-4.17 (m, 5H), 4.05-4.01 (m, 1H), 3.94-3.86 (m, 1H), 3.82-3.70 (m, 3H), 3.30-3.16 (m, 1H). LRMS (ESI-) calcd for C₁₅H₂₅N₃O₁₆P₂ (M−H⁺) 564.06, found 564.10.

General Protocol for Synthesis of Activated Esters

To a solution of carboxylic acid was added dicyclohexylcarbodiimide (1.1 equiv) and N-hydroxysuccinimide (1.2 equiv) and the resulting suspension was stirred overnight followed by vacuum filtration. The filtrate was concentrated and dissolved in EtOAc followed by washing with saturated NaHCO₃ and brine. The organic layer was dried over Na₂SO₄, filtrated and concentrated in vacuo to use crude in the next reaction.

General Protocol for Attaching Activated Esters to UDP-D-Galactosamine (20)

UDP-D-galactosamine (20) was dissolved in 0.1 M NaHCO₃ (0.2 M) and activated ester (2 equiv) dissolved in DMF (0.2 M) was added. The reaction was stirred overnight at r.t. and concentrated in vacuo. Flash chromatography (7:2:1-5:2:1 EtOAc:MeOH:H₂O) afforded the product.

### Example 5

### Synthesis of 3-pyridylcarbonyl Derivative of UDP-GalNH₂ (21)

3-Nicotinic acid (200 mg, 1.6 mmol) was converted into the active ester according the standard protocol to yield the activated ester in crude form.

¹H-NMR (CDCl₃): δ 9.32-9.31 (m, 1H), 8.89-8.87 (m, 1H), 8.40-8.38 (m, 1H), 7.49-7.45 (m, 1H), 2.91 (s, 4H).

Next, UDP-galactosamine 20 (50 mg, 0.09 mmol) was reacted with the active ester derivative of 3-nicotinic acid (37 mg, 0.18 mmol) according the standard protocol to yield UDP-galactosamine variant 21 (1.5 mg, 0.0022 mmol, 2.5%).

LRMS (ESI-) calcd for C₂₁H₂₈N₄O₁₇P₂ (M−H⁺) 669.09, found 669.1.

### Example 5-1

### Synthesis of 6-azidonicotinic Acid Derivative of UDP-GalNH₂ (21b)

6-chloronicotinic acid (1 g, 6.5 mmol) was dissolved in EtOH (7 mL) and water (2 mL) followed by the addition of NaN₃ (420 mg, 7.2 mmol). The reaction was heated to 85° C. and after stirring overnight the mixture was concentrated under reduced pressure. 6-Azidonicotinic acid was isolated as a mixture with NaCl and NaN₃ and used crude. ¹H-NMR (400 MHz, DMSO-d6): δ 8.90 (dd, J=0.8 Hz, J=2.4 Hz, 1H), 8.29 (dd, J=2.4 Hz, J=8.0 Hz, 1H), 7.67 (dd, J=0.8 Hz, J=8.0 Hz, 1H). Next, to crude 6-azidonicotinic acid (about 6.5 mmol) in DCM (60 mL) was added N-hydroxysuccinimide (853 mg, 7.2 mmol) and EDC.HCl (1.5 g, 7.8 mmol) and the reaction was stirred for 3 h followed by the addition of water (60 mL). The organic layer was washed with water (2×60 mL), dried over Na₂SO₄, filtrated and concentrated under reduced pressure. The product 2,5-dioxopyrrolidin-1-yl 6-azidonicotinate was used without further purification in the next reaction.

UDP-GalNH₂ (20, 50 mg, 0.09 mmol) was dissolved in 0.1 M NaHCO₃ (1 mL) and 2,5-dioxopyrrolidin-1-yl 6-azidonicotinate (95 mg, 0.35 mmol) dissolved in DMF (2 mL), was added. The reaction was stirred overnight at r.t. and concentrated in vacuo. Flash chromatography (7:2:1-3:2:1 EtOAc:MeOH:H₂O) afforded 21b (23 mg, 0.03 mmol, 37%). LRMS (ESI-) calcd for C₂₁H₂₆N₇O₁₇P₂ (M−H⁺) 710.09, found 710.14.

### Example 6

### Synthesis of Furylcarbonyl Derivative of UDP-GalNH₂ (22)

According to the standard protocol furan-2-carboxylic acid (162 mg, 1.45 mmol) was reacted with N-hydroxysuccinimide to yield the desired ester.

¹H-NMR (CDCl₃): δ 7.74-7.73 (m, 1H), 8.89-8.87 (m, 1H), 7.50-7.49 (m, 1H), 6.64-6.62 (m, 1H), 2.90 (s, 4H).

Next, UDP galactosamine 20 (50 mg, 0.09 mmol) was reacted with furan-2-carboxylic acid succinimidyl ester (37 mg, 0.18 mmol) according the standard protocol to yield UDP-galactosamine variant 22 (3 mg, 0.0045 mmol, 5%). LRMS (ESI-) calcd for C₂₀H₂₇N₃O₁₈ P₂ (M−H⁺) 658.01, found 658.1.

### Example 7

### Synthesis of 4-azido-3,5-difluorobenzoyl Derivative of UDP-GalNH₂ (23)

4-Azido-3,5-difluorobenzoic acid succinimidyl ester was prepared according to the procedure for pent-4-ynoic acid succinimidyl ester according to Rademann et al., Angew. Chem. Int. Ed., 2012, 51, 9441-9447.

Thus, to a solution of 4-azido-3,5-difluorobenzoic acid was added dicyclohexylcarbodiimide (1.1 equiv) and N-hydroxysuccinimide (1.2 equiv) and the resulting suspension was stirred overnight followed by vacuum filtration. The filtrate was concentrated and dissolved in EtOAc followed by washing with saturated NaHCO₃ and brine. The organic layer was dried over Na₂SO₄, filtrated and concentrated in vacuo to use crude in the next reaction.

¹H-NMR (300 MHz, CDCl₃): δ 7.74-7.66 (m, 2H), 2.91 (s, 4H).

Next, UDP-GalNH₂ (20, 30 mg, 0.0531 mmol) was dissolved in 0.1 M NaHCO₃ (0.2 M) and the N-hydroxysuccinimide ester of 4-azido-3,5-difluorobenzoic acid (31 mg, 0.106 mmol, 2 equiv.), dissolved in DMF (0.2 M), was added. The reaction was stirred overnight at r.t. and concentrated in vacuo. Flash chromatography (7:2:1-5:2:1 EtOAc:MeOH:H₂O) afforded the product 23 (8 mg, 0.0107 mmol, 20%).

¹H-NMR (300 MHz, D₂O): δ 7.73 (d, J=8.4 Hz, 1H), 7.52-7.31 (m, 2H), 5.87-5.71 (m, 2H), 5.65-5.57 (m, 1H), 5.47-5.33 (m, 1H), 4.43-3.96 (m, 8H), 3.76-3.60 (m, 2H).

LRMS (ESI-) calcd for C₂₂H₂₅F₂N₆O₁₇P₂ (M−H⁺) 745.07, found 744.9.

### Example 8

### Synthesis of 4-azido-2,3,5,6-tetrafluorobenzoyl Derivative of UDP-GalNH₂ (23b)

UDP-GalNH₂ (20, 41 mg, 0.073 mmol) was dissolved in 0.1 M NaHCO₃ (0.2 M) and the N-hydroxysuccinimide ester of 4-azido-2,3,5,6-difluorobenzoic acid (N₃-TFBA OSu ester, commercially available from Iris-Biotech) (47 mg, 0.0.145 mmol, 2 equiv.), dissolved in DMF (0.2 M), was added. The reaction was stirred overnight at r.t. and concentrated in vacuo. Flash chromatography (8:2:1-5:2:1 EtOAc:MeOH:H₂O) afforded the 4-azido-2,3,5,6-tetrafluorobenzoyl derivative of UDP-GalNH₂.

LRMS (ESI-) calcd for C₂₂H₂₃F₄N₆O₁₇P₂ (M−H⁺) 781.05, found 781.0.

### Example 9

### Synthesis of Cyclopentylcarbonyl Derivative of UDP-GalNH₂ (24)

Cyclopentanecarboxylic acid (84.2 mg, 0.38 mmol) was reacted according the standard protocol to yield the crude activated ester.

¹H-NMR (CDCl₃): δ 3.07-2.96 (m, 1H), 2.79 (s, 4H), 2.01-1.93 (m, 4H), 1.75-1.59 (m, 4H).

Next, UDP-galactosamine 20 (50 mg, 0.09 mmol) was reacted with cyclopentanecarboxylic acid succinimidyl ester (37 mg, 0.18 mmol) according the standard protocol to yield UDP-galactosamine variant 24 (6 mg, 0.009 mmol, 10%).

### Example 9-1

### Synthesis of Benzoyl Derivative of UDP-GalNH₂ (32)

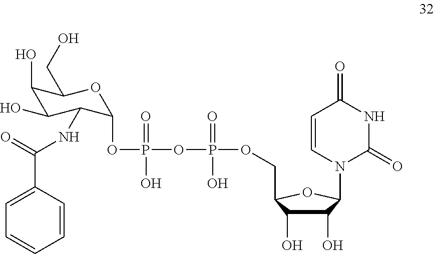

To a solution of benzoic acid (500 mg, 4.09 mmol) in DCM (20 mL) was added EDCI (1.18 g, 6.14 mmol) and NHS (707 mg, 6.14 mmol) and the reaction mixture was stirred for 1 h at r.t. The solution was washed with H₂O (3×10 mL), the organic layer dried over sodium sulfate and concentrated in vacuo to afford the OSu-ester product (778 mg, 3.55 mmol, 87%).

¹H-NMR (400 MHz, CDCl₃): δ 8.16-8.12 (m, 2H), 7.71-766 (m, 1H), 7.55-7.49 (m, 2H), 2.91 (br s, 4H) ppm.

UDP-D-galactosamine 20 (55 mg, 0.0972 mmol) was dissolved in 1 mL 0.1 M NaHCO₃ and benzoic acid succinimidyl ester (107 mg, 0.486 mmol), dissolved in 1 mL DMF, was added. The reaction was allowed to stir at r.t. overnight. Product formation was confirmed by LCMS analysis.

LRMS (ESI-) calcd for C₂₂H₂₉N₃O₁₇P₂ (M−H⁺) 668.09, found 668.01.

Mass Spectral Analysis of Fabricator™-Digested Monoclonal Antibodies

A solution of 20 μg (modified) IgG was incubated for 1 hour at 37° C. with Fabricator™ (commercially available from Genovis, Lund, Sweden) (1.25 U/μL) in phosphate-buffered saline (PBS) pH 6.6 in a total volume of 10 μL. Fabricator™-digested samples were washed trice with milliQ using an Amicon Ultra-0.5, Ultracel-10 Membrane (Millipore) resulting in a final sample volume of approximately 40 μL. The Fc/2 fragment was analyzed by electrospray ionization time-of-flight (ESI-TOF) on a JEOL AccuTOF. Deconvoluted spectra were obtained using Magtran software.

### Example 10

### Preparation of Deglycosylated Trastuzumab (Trimmed Trastuzumab) by Endo S Treatment

Glycan trimming of trastuzumab (27) was performed with Endo S from Streptococcus pyogenes (commercially available from Genovis, Lund, Sweden). Thus, trastuzumab (10 mg/mL) was incubated with Endo S (40 U/mL) in 25 mM Tris pH 8.0 for approximately 16 hours at 37° C. The deglycosylated IgG was concentrated and washed with 10 mM MnCl₂ and 25 mM Tris-HCl pH 8.0 using an Amicon Ultra-0.5, Ultracel-10 Membrane (Millipore).

After deconvolution of peaks, the mass spectrum showed one peak of the light chain and two peaks of the heavy chain. The two peaks of heavy chain belonged to one major product (49496 Da, 90% of total heavy chain), resulting from core GlcNAc(Fuc) substituted trastuzumab, and a minor product (49351 Da, ±10% of total heavy chain), resulting from deglycosylated trastuzumab.

### Example 10-1

### Preparation of Deglycosylated Cetuximab (Trimmed Cetuximab) by Endo S Treatment

Glycan trimming of cetuximab was performed with Endo S from Streptococcus pyogenes (commercially available from Genovis, Lund, Sweden). Thus, cetuximab (10 mg/mL) was incubated with Endo S (0.01 mg/mL) in 25 mM Tris-HCL pH 7.5 and 150 mM NaCl for approximately 4 hours at 37° C. The deglycosylated IgG was concentrated and washed with 10 mM MnCl₂ and 25 mM Tris-HCl pH 8.0 using an Amicon Ultra-0.5, Ultracel-10 Membrane (Millipore).

Mass spectral analysis of the Fabricator™-digested sample showed four peaks of the Fc/2-fragment belonging to one major product (observed mass 24138 Da, calculated mass of 24136 Da, approximately 80% of total Fc/2 fragment), corresponding to core GlcNAc(Fuc)-substituted cetuximab, and three minor products (observed masses of 23994, 24266 and 25008 Da, approximately 5, 10 and 5% of total Fc/2 fragment), corresponding to core GlcNAc-substituted cetuximab, core GlcNAc(Fuc)-substituted cetuximab with C-terminal lysine and Man₅-GlcNAc-GlcNAc(Fuc)-substituted cetuximab.

### Example 10-2

### Preparation of Deglycosylated Bevacizumab by Endo S Treatment

Glycan trimming of bevacizumab was performed with Endo S from Streptococcus pyogenes (commercially available from Genovis, Lund, Sweden). Thus, bevacizumab (10 mg/mL) was incubated with Endo S (0.01 mg/mL) in 25 mM Tris-HCL pH 7.5 and 150 mM NaCl for approximately 4 hours at 37° C. The deglycosylated IgG was concentrated and washed with 10 mM MnCl₂ and 25 mM Tris-HCl pH 8.0 using an Amicon Ultra-0.5, Ultracel-10 Membrane (Millipore).

Mass spectral analysis of the Fabricator™-digested sample showed one major peaks of the Fc/2-fragment (observed mass 24139 Da, calculated mass of 24136 Da, approximately 95% of total Fc/2 fragment), corresponding to core GlcNAc(Fuc)-substituted bevacizumab.

### Example 10-3

### Preparation of Deglycosylated Adalimumab by Endo S Treatment

Glycan trimming of cetuximab was performed with Endo S from Streptococcus pyogenes (commercially available from Genovis, Lund, Sweden). Thus, adalimumab (10 mg/mL) was incubated with Endo S (0.01 mg/mL) in 25 mM Tris-HCL pH 7.5 and 150 mM NaCl for approximately 4 hours at 37° C. The deglycosylated IgG was concentrated and washed with 10 mM MnCl₂ and 25 mM Tris-HCl pH 8.0 using an Amicon Ultra-0.5, Ultracel-10 Membrane (Millipore).

Mass spectral analysis of the Fabricator™-digested sample showed a complete conversion of the adalimumab starting material (observed mass 25203 Da, approximately 90% of total Fc/2 fragment), corresponding to either (Gal-GlcNAc)₂-Man₃-GlcNAc₂- or (Gal-GlcNAc)₂-Man₃-GlcNAc-GlcNAc(Fuc)-substituted adalimumab, into the product (24107 Da, approximately 90% of total Fc/2 fragment), corresponding to either the GlcNAc- or the GlcNAc(Fuc)-substituted adalimumab.

General Protocol for Glycosyltransfer of Galactose Derivative UDP-GalNAz 21-24 with Gal-T1(Y289L) (Expressed in E. coli). See FIG. 7.

Enzymatic introduction of UDP-Gal derivatives 21-24 onto deglycosylated trastuzumab was effected with a mutant of bovine β(1,4)-galactosyltransferase [β(1,4)-Gal-T1(Y289L)] (expressed in E. coli). The deglycosylated trastuzumab (10 mg/mL) was incubated with the appropriate UDP-galactose derivative (0.4 mM) and β(1,4)-Gal-T1(Y289L) (1 mg/mL) in 10 mM MnCl₂ and 25 mM Tris-HCl pH 8.0 for 16 hours at 30° C.

Next, the functionalized trastuzumab was incubated with protein A agarose (40 μL per mg IgG) for 2 hours at 4° C. The protein A agarose was washed three times with PBS and the IgG was eluted with 100 mM glycine-HCl pH 2.7. The eluted IgG was neutralized with 1 M Tris-HCl pH 8.0 and concentrated and washed with PBS using an Amicon Ultra-0.5, Ultracel-10 Membrane (Millipore) to a concentration of 15-20 mg/mL.

### Example 11

### Glycosyltransfer of 3-pyridylcarbonyl Derivative of UDP-Galactosamine to Trimmed Trastuzumab Under the Action of Gal-T1(Y289L) (Expressed in E. coli)

Trimmed trastuzumab (10 mg/mL, 3.3 nmol), obtained by Endo S treatment of trastuzumab, was incubated with UDP-galactosamine variant 21 (2.5 mM) and β(1,4)-Gal-T1(Y289L) (0.68 mg/mL, 45 μL) in 10 mM MnCl₂ and 25 mM Tris-HCl pH 8.0 at 30° C. overnight.

Mass spectral analysis of the reduced sample indicated the formation of the product (49764.1 Da, approximately 5% of total heavy chain), resulting from galacosamide nicotinic acid transfer to core GlcNAc(Fuc) substituted trastuzumab heavy chain.

### Example 11-1

### Glycosyltransfer of 3-pyridylcarbonyl Derivative of UDP-Galactosamine to Trimmed Trastuzumab Under the Action of Gal-T1(Y289L,C342T) (Expressed in E. coli)

Trimmed trastuzumab (10 mg/mL, 3.3 nmol), obtained by Endo S treatment of trastuzumab, was incubated with nicotinic acid variant of UDP-galactosamine (21, 2 mM) and Gal-T1(Y289L,C342T) (0.5 mg/mL, 3 μL/4 mg/ml) in 10 mM MnCl₂ and 25 mM Tris-HCl pH 7.5 at 30° C. overnight. Mass spectral analysis of the sample after treatment with Fabricator™ (commercially available from Genovis) indicated the formation of the correct structure of C_{H}2-C_{H}3 fragment of 28 (24404 Da, expected mass 24405 Da, approximately 35% conversion), resulting from galactosamide nicotinic acid transfer to core GlcNAc(Fuc) substituted trastuzumab heavy chain.

### Example 12

### Glycosyltransfer of Furylcarbonyl Derivative of UDP-Galactosamine to Trimmed Trastuzumab Under the Action of Gal-T1(Y289L) (Expressed in E. coli)

Trimmed trastuzumab (10 mg/mL, 1.3 nmol), obtained by Endo S treatment of trastuzumab, was incubated with UDP-galactosamine variant 22 (4 mM) and β(1,4)-Gal-T1(Y289L) (1.4 mg/mL, 10 μL) in 10 mM MnCl₂ and 25 mM Tris-HCl pH 8.0 at 30° C. overnight. Mass spectral analysis of the reduced sample indicated the formation of the product (49750.9 Da, approximately 80% of total heavy chain), resulting from galactosamide furan-2-carboxyl acid transfer to core GlcNAc(Fuc) substituted trastuzumab heavy chain as shown in FIG. 9.

### Example 13

### Glycosyltransfer of 4-azido-3,5-difluorobenzoyl Derivative of UDP-Galactosamine to Trimmed Trastuzumab Under the Action of Gal-T1(Y289L) (Expressed in E. coli)

Trimmed trastuzumab (10 mg/mL, 6.6 nmol), obtained by Endo S treatment of trastuzumab, was incubated with the 4-azido-3,5-difluorobenzoyl derivative of UDP-galactosamine (23, 7 mM) and β(1,4)-Gal-T1(Y289L) (2 mg/mL) in 10 mM MnCl₂ and 25 mM Tris-HCl pH 8.0 at 30° C. overnight. Mass spectral analysis of the reduced sample indicated the formation of a one major product (49813 Da, approximately 90% of total heavy chain), resulting from transfer of 23 to core GlcNAc(Fuc)-substituted trastuzumab heavy chain.

FIG. 8 shows the heavy chain of trimmed trastuzumab (upper spectrum) and the heavy chain of trastuzumab conjugated to (lower spectrum).

### Example 14

### Glycosyltransfer of Cyclopentylcarbonyl Derivative of UDP-Galactosamine to Trimmed Trastuzumab Under the Action of Gal-T1(Y289L) (Expressed in E. coli)

Trimmed trastuzumab (10 mg/mL, 1.3 nmol), obtained by Endo S treatment of trastuzumab, was incubated with UDP-galactosamine variant 24 (4 mM) and (3(1,4)-Gal-T1(Y289L) (1.4 mg/mL, 10 μL) in 10 mM MnCl₂ and 25 mM Tris-HCl pH 8.0 at 30° C. overnight.

Mass spectral analysis of the reduced sample indicated the formation of the product (49753.7 Da, approximately 90% of total heavy chain), resulting from galacosamide cyclopentanecarboxylic acid transfer to core GlcNAc(Fuc) substituted trastuzumab heavy chain.

### Example 15

### Cloning and Expression of Gal-T1 Mutants Y289N, Y289F, Y289M, Y289V, Y289A and Y289G and Y289I. (Expressed in E. coli)

The GalT mutant genes were amplified from a construct containing the sequence encoding the catalytic domain of GalT consisting of 130-402 aa residues, by the overlap extension PCR method. The wild type enzyme is represented by SEQ ID NO: 17. For Y289N mutant (represented by aa sequence 130-402 from SEQ ID NO: 18), the first DNA fragment was amplified with a pair of primers: Oligo38_GalT_External_Fw (CAG CGA CAT ATG TCG CTG ACC GCA TGC CCT GAG GAG TCC represented by SEQ ID NO: 1) and Oligo19_GalT_Y289N_Rw (GAC ACC TCC AAA GTT CTG CAC GTA AGG TAG GCT AAA represented by SEQ ID NO: 2). The NdeI restriction site is underlined, while the mutation site is highlighted in bold. The second fragment was amplified with a pair of primers: Oligo29_GalT_External_Rw (CTG ATG GAT GGA TCC CTA GCT CGG CGT CCC GAT GTC CAC represented by SEQ ID NO: 3) and Oligo18_GalT_Y289N_Fw (CCT TAC GTG CAG AAC TTT GGA GGT GTC TCT GCT CTA represented by SEQ ID NO: 4). The BamHI restriction site is underlined, while the mutation site is highlighted in bold. The two fragments generated in the first round of PCR were fused in the second round using Oligo38_GalT_External_Fw and Oligo29_GalT_External_Rw primers. After digestion with NdeI and BamHI. This fragment was ligated into the pET16b vector cleaved with the same restriction enzymes. The newly constructed expression vector contained the gene encoding Y289N mutant and the sequence encoding for the His-tag from pET16b vector, which was confirmed by DNA sequencing results. For the construction of Y289F (represented by aa sequence 130-402 from SEQ ID NO: 19), Y289M (represented by aa sequence 130-402 from SEQ ID NO: 20), Y289I (represented by aa sequence 130-402 from SEQ ID NO: 21), Y289V (represented by aa sequence 130-402 from SEQ ID NO: 22), Y289A (represented by aa sequence 130-402 from SEQ ID NO: 23) and Y289G (represented by aa sequence 130-402 from SEQ ID NO: 24) mutants the same procedure was used, with the mutation sites changed to TTT, ATG, ATT, GTG, GCG or GGC triplets encoding for phenylalanine, methionine, isoleucine, valine, alanine or glycine, respectively. More specifically, for the construction of Y289F the first DNA fragment was amplified with a pair of primers defined herein as SEQ ID NO: 1 and SEQ ID NO: 5 and the second fragment was amplified with a pair of primers defined herein as SEQ ID NO: 3 and SEQ ID NO: 6 (be referred to Table 1 for the related sequences). Furthermore, for the construction of Y289M the first DNA fragment was amplified with a pair of primers defined herein as SEQ ID NO: 1 and SEQ ID NO: 7 and the second fragment was amplified with a pair of primers defined herein as SEQ ID NO: 3 and SEQ ID NO: 8. For the construction of Y289I the first DNA fragment was amplified with a pair of primers defined herein as SEQ ID NO: 1 and SEQ ID NO: 9 and the second fragment was amplified with a pair of primers defined herein as SEQ ID NO: 3 and SEQ ID NO: 10. For the construction of Y289V the first DNA fragment was amplified with a pair of primers defined herein as SEQ ID NO: 1 and SEQ ID NO: 11 and the second fragment was amplified with a pair of primers defined herein as SEQ ID NO: 3 and SEQ ID NO: 12. for the construction of Y289A the first DNA fragment was amplified with a pair of primers defined herein as SEQ ID NO: 1 and SEQ ID NO: 13 and the second fragment was amplified with a pair of primers defined herein as SEQ ID NO: 3 and SEQ ID NO: 14. For the construction of Y289G the first DNA fragment was amplified with a pair of primers defined herein as SEQ ID NO: 1 and SEQ ID NO: 15 and the second fragment was amplified with a pair of primers defined herein as SEQ ID NO: 3 and SEQ ID NO: 16 (be referred to Table 1 for the related sequences).

GalT mutants were expressed, isolated and refolded from inclusion bodies according to the reported procedure by Qasba et al. (Prot. Expr. Pur. 2003, 30, 219-229). After refolding, the precipitate was removed and the soluble and folded protein was isolated using a Ni-NTA column (HisTrap excel 1 mL column, GE Healthcare). After elution with 25 mM Tris-HCl pH 8.0, 300 mM NaCl and 200 mM imidazole, the protein was dialyzed against 25 mM Tris-HCl pH 8.0 and concentrated to 2 mg/mL using a spinfilter (Amicon Ultra-15 Centrifugal Filter Unit with Ultracel-10 membrane, Merck Millipore).

TABLE 1
Sequence identification of the primers used

| SEQ ID NO | Nucleotide sequence |
|---|---|
| SEQ ID NO: 1 | CAG CGA CAT ATG TCG CTG ACC GCA |
| | TGC CCT GAG GAG TCC |
| SEQ ID NO: 2 | GAC ACC TCC AAA GTT CTG CAC GTA |
| | AGG TAG GCT AAA |
| SEQ ID NO: 3 | CTG ATG GAT GGA TCC CTA GCT CGG |
| | CGT CCC GAT GTC CAC |
| SEQ ID NO: 4 | CCT TAC GTG CAG AAC TTT GGA GGT |
| | GTC TCT GCT CTA |
| SEQ ID NO: 5 | GAC ACC TCC AAA AAA CTG CAC GTA |
| | AGG TAG GCT AAA |
| SEQ ID NO: 6 | CCT TAC GTG CAG TTT TTT GGA GGT |
| | GTC TCT GCT CTA |
| SEQ ID NO: 7 | GAC ACC TCC AAA CAT CTG CAC GTA |
| | AGG TAG GCT AAA |
| SEQ ID NO: 8 | CCT TAC GTG CAG ATG TTT GGA GGT |
| | GTC TCT GCT CTA |
| SEQ ID NO: 9 | GAC ACC TCC AAA AAT CTG CAC GTA |
| | AGG TAG GCT AAA |
| SEQ ID NO: 10 | CCT TAC GTG CAG ATT TTT GGA GGT |
| | GTC TCT GCT CTA |
| SEQ ID NO: 11 | GAC ACC TCC AAA CAC CTG CAC GTA |
| | AGG TAG GCT AAA |
| SEQ ID NO: 12 | CCT TAC GTG CAG GTG TTT GGA GGT |
| | GTC TCT GCT CTA |
| SEQ ID NO: 13 | GAC ACC TCC AAA CGC CTG CAC GTA |
| | AGG TAG GCT AAA |
| SEQ ID NO: 14 | CCT TAC GTG CAG GCG TTT GGA GGT |
| | GTC TCT GCT CTA |
| SEQ ID NO: 15 | GAC ACC TCC AAA GCC CTG CAC GTA |
| | AGG TAG GCT AAA |
| SEQ ID NO: 16 | CCT TAC GTG CAG GGC TTT GGA GGT |
| | GTC TCT GCT CTA |

### Example 16

### Expression and Refolding of Gal-T1(Y289L,C342T) from E. coli

A pET22b vector containing the sequence encoding residues 130-402 of bovine Gal-T1 with the Y289L and C342T mutations between the NdeI-BamHI sites was obtained from Genscript. Using this plasmid Gal-T1(Y289L,C342T) was expressed, isolated and refolded from inclusion bodies according to the reported procedure by Qasba et al. (Prot. Expr. Pur. 2003, 30, 219-76229, incorporated by reference herein). After refolding, the solution was dialyzed against 20 mM Tris pH 7.5 and the insoluble protein was removed by centrifugation (10 minutes 10.000 g). The soluble Gal-T1(Y289L,C342T), represented by SEQ ID NO: 25, was purified and concentrated using a cation exchange column (Source 15S HR16/10 column, GE Healthcare). After elution with 20 mM Tris-HCl pH 7.5, 1 M NaCl, the protein was dialyzed against 20 mM Tris-HCl pH 7.5. This procedure yielded 90 mg inclusion bodies from 0.5 L culture, which after refolding gave 3.9 mg active soluble protein.

### Example 17

### Expression of Gal-T1 Mutants Y289L, Y289F, Y289M, Y289V, Y289A and Y289G and of Gal-T1 Double Mutants Y289L,C342T and Y289M,C342T in CHO and Purification Thereof

A set of Gal-T 1 mutants encoding residues 74-402 of bovine Gal-T 1 were transiently expressed in CHO K1 cells by Evitria (Zurich, Switzerland) which include the Gal-T1 single mutants Y289L (represented by SEQ ID NO: 26), Y289F (represented by SEQ ID NO: 27), Y289M (represented by SEQ ID NO: 28), Y289V (represented by SEQ ID NO: 29), Y289A (represented by SEQ ID NO: 30), and Y289G (represented by SEQ ID NO: 31), and the Gal-T1 double mutants Y289L,C342T (represented by SEQ ID NO: 32), and Y289M,C342T (represented by SEQ ID NO: 33). The mutants were purified using a cation exchange column (Source 15S HR16/10 column, GE Healthcare) as described above. Purified proteins were analyzed by SDS-PAGE.

### Example 18

### Glycosyltransfer of 4-azido-3,5-difluorobenzoyl Derivative of UDP-Galactosamine to Trimmed Trastuzumab Under the Action of Gal-T1 Mutants Y289L, Y289M, Y289A or Y289G (Expressed in CHO)

Trimmed trastuzumab (10 mg/mL, 66 obtained by Endo S treatment of trastuzumab, was incubated with the 4-azido-3,5-difluorobenzoyl derivative of UDP-galactosamine (23, 5 mM) and one of the Gal-T1 single mutants Y289L, Y289M, Y289A or Y289G (expressed in CHO as described above) (0.5 mg/mL) in 10 mM MnCl2 and 25 mM Tris-HCl pH 8.0 at 30° C. overnight. Mass spectral analysis of the reduced sample indicated a partial conversion of the core GlcNac(Fuc)-substituted trastuzumab heavy chain (49504 Da) into product 30 (49818 to 49825 Da, 20 to 50% of total heavy chain depending on the Gal-T1 mutant used), resulting from transfer of 23 to core GlcNAc(Fuc)-substituted trastuzumab heavy chain. The observed conversion was approximately 20% for Gal-T1(Y289A) and Gal-T1(Y289G), approximately 30% for Gal-T1(Y289L) and approximately 50% for Gal-T1(Y289M).

### Example 19

### Glycosyltransfer of 4-azido-3,5-difluorobenzoyl Derivative of UDP-Galactosamine to Trimmed Trastuzumab Under the Action of Gal-T1(Y289L,C342T)

Trimmed trastuzumab (10 mg/mL, 66 obtained by Endo S treatment of trastuzumab, was incubated with the 4-azido-3,5-difluorobenzoyl derivative of UDP-galactosamine (23, 1 mM) and Gal-T1(Y289L,C342T) (1.0 mg/mL) in 10 mM MnCl2 and 25 mM Tris-HCl pH 8.0 at 30° C. overnight. Mass spectral analysis of the reduced sample indicated a complete conversion of core GlcNac(Fuc)-substituted trastuzumab (observed mass 49502 Da for the heavy chain, calculated mass of 49506 Da) into the product 30 (observed mass 49818 Da, calculated mass of 49822 Da for the reduced product), resulting from transfer of 23 to core GlcNAc(Fuc)-substituted trastuzumab heavy chain followed by reduction of the azide during sample preparation.

### Example 19-1

### Glycosyltransfer of 4-azido-3,5-difluorobenzoyl Derivative of UDP-Galactosamine to Trimmed Trastuzumab Under the Action of Gal-T1(Y289L,C342T) (Expressed in CHO)

Trimmed trastuzumab (10 mg/mL, 66 obtained by Endo S treatment of trastuzumab, was incubated with the 4-azido-3,5-difluorobenzoyl derivative of UDP-galactosamine (23, 5 mM) and Gal-T1(Y289L,C342T) (expressed in CHO as described above) (2.0 mg/mL) in 10 mM MnCl2 and 25 mM Tris-HCl pH 8.0 at 30° C. overnight. Mass spectral analysis of the Fabricator™-digested sample indicated a complete conversion of core GlcNac(Fuc)-substituted trastuzumab (observed mass 24139 Da, calculated mass of 24136 Da) into the product 30 (observed mass 24481 Da, calculated mass of 24479 Da), resulting from transfer of 23 to core GlcNAc(Fuc)-substituted trastuzumab.

### Example 19-2

### Glycosyltransfer of 4-azido-3,5-difluorobenzoyl Derivative of UDP-Galactosamine to Trimmed Trastuzumab Under the Action of Gal-T1(Y289M,C342T) (Expressed in CHO)

Trimmed trastuzumab (10 mg/mL, 66 obtained by Endo S treatment of trastuzumab, was incubated with the 4-azido-3,5-difluorobenzoyl derivative of UDP-galactosamine (23, 5 mM) and Gal-T1(Y289M,C342T) (expressed in CHO as described above) (1.0 mg/mL) in 10 mM MnCl₂ and 25 mM Tris-HCl pH 8.0 at 30° C. overnight. Mass spectral analysis of the Fabricator™-digested sample indicated a complete conversion of core GlcNac(Fuc)-substituted trastuzumab (observed mass 24139 Da, calculated mass of 24136 Da) into the product 30 (observed mass 24481 Da, calculated mass of 24479 Da), resulting from transfer of 23 to core GlcNAc(Fuc)-substituted trastuzumab.

### Example 20

### Glycosyltransfer of 4-azido-2,3,5,6-tetrafluorobenzoyl Derivative of UDP-Galactosamine to Trimmed Trastuzumab Under the Action of Gal-T1(Y289L,C342T), or Under the Action of Gal-T1(Y289L,C342T) or Gal-T1(Y289M,C342T), Expressed in CHO.

Trimmed trastuzumab (10 mg/mL, 66 μM), obtained by Endo S treatment of trastuzumab, was incubated with the 4-azido-2,3,5,6-tetrafluorobenzoyl derivative of UDP-galactosamine (23b, 1 mM) and Gal-T1(Y289L,C342T) (1.0 mg/mL) in 10 mM MnCl2 and 25 mM Tris-HCl pH 8.0 at 30° C. overnight. Mass spectral analysis of the Fabricator™-digested sample indicated a partial conversion of core GlcNac(Fuc)-substituted trastuzumab (observed mass 24139 Da, calculated mass of 24136 Da) into the product 30b (observed mass 24518 Da, calculated mass of 24514 Da, approximately 10% of total Fc/2 fragment), resulting from transfer of 23b to core GlcNAc(Fuc)-substituted trastuzumab.

The reaction with Gal-T1(Y289L,C342T) (expressed in CHO as described above) was performed using exactly the same conditions, which indicated approximately 70% conversion into product 30b according to mass spectral analysis of the Fabricator™-digested sample (observed mass 24518 Da, calculated mass of 24514 Da). For Gal-T1(Y289M,C342T) (expressed in CHO as described above) the reaction was performed using similar conditions except for a 5-fold higher concentration of the 4-azido-2,3,5,6-tetrafluorobenzoyl derivative of UDP-galactosamine (23b, 5 mM), which indicated approximately 10% conversion into product 30b according to mass spectral analysis of the Fabricator™-digested sample (observed mass 24518 Da, calculated mass of 24514 Da).

### Example 20-1

### Glycosyltransfer of 4-azido-2,3,5,6-tetrafluorobenzoyl Derivative of UDP-Galactosamine to Trimmed Cetuximab Under the Action of Gal-T1(Y289L,C342T)

Trimmed cetuximab (5 mg/mL, 33 μM), obtained by Endo S treatment of cetuximab, was incubated with the 4-azido-2,3,5,6-tetrafluorobenzoyl derivative of UDP-galactosamine (23b, 2 mM) and Gal-T1(Y289L,C342T) (1.0 mg/mL) in 25 mM Tris-HCL pH 7.5 and 150 mM NaCl at 30° C. overnight. Mass spectral analysis of the Fabricator™-digested sample indicated a partial conversion of the core GlcNac(Fuc)-substituted adalimumab (observed mass 24138 Da, calculated mass of 24136 Da) into product 30b (observed mass 24518 Da, calculated mass of 24514 Da, approximately 20% of total Fc/2 fragment), resulting from transfer of 23b to core GlcNAc(Fuc)-substituted adalimumab.

### Example 20-2

### Glycosyltransfer of 4-azido-2,3,5,6-tetrafluorobenzoyl Derivative of UDP-Galactosamine to Trimmed Bevacizumab Under the Action of Gal-T1(Y289L,C342T)

Trimmed bevacizumab (5 mg/mL, 33 μM), obtained by Endo S treatment of bevacizumab, was incubated with the 4-azido-2,3,5,6-tetrafluorobenzoyl derivative of UDP-galactosamine (23b, 2 mM) and Gal-T1(Y289L,C342T) (1.0 mg/mL) in 25 mM Tris-HCL pH 7.5 and 150 mM NaCl at 30° C. overnight. Mass spectral analysis of the Fabricator™-digested sample indicated a partial conversion of core GlcNac(Fuc)-substituted bevacizumab (observed mass 24139 Da, calculated mass of 24136 Da) into product 30b (observed mass 24517 Da, calculated mass of 24514 Da, approximately 30% of total Fc/2 fragment), resulting from transfer of 23b to core GlcNAc(Fuc)-substituted bevacizumab.

### Example 20-3

### Glycosyltransfer of 4-azido-2,3,5,6-tetrafluorobenzoyl Derivative of UDP-Galactosamine to Trimmed Adalimumab Under the Action of Gal-T1(Y289L,C342T)

Trimmed adalimumab (5 mg/mL, 33 μM), obtained by Endo S treatment of adalimumab, was incubated with the 4-azido-2,3,5,6-tetrafluorobenzoyl derivative of UDP-galactosamine (23b, 2 mM) and Gal-T1(Y289L,C342T) (1.0 mg/mL) in 25 mM Tris-HCL pH 7.5 and 150 mM NaCl at 30° C. overnight. Mass spectral analysis of the Fabricator™-digested sample indicated a partial conversion of the core GlcNac(Fuc)- or GlcNAc-substituted adalimumab (observed mass 24107 Da) into product 30b (observed mass 24485 Da, approximately 30% of total Fc/2 fragment), resulting from transfer of 23b to core GlcNAc(Fuc)- or GlcNAc-substituted adalimumab.

### Example 21

### Glycosyltransfer of 6-azidonicotinic Acid Derivative of UDP-Galactosamine to Trimmed Trastuzumab Under the Action of Gal-T1(Y289L,C342T)

Trimmed trastuzumab (10 mg/mL, 66 μM), obtained by Endo S treatment of trastuzumab, was incubated with the 6-azido-nicotinic acid derivative of UDP-galactosamine (21b, 5 mM) and Gal-T1(Y289L,C342T) (1.0 mg/mL) in 10 mM MnCl₂ and 25 mM Tris-HCl pH 7.5 at 30° C. overnight. Mass spectral analysis of the Fabricator™-digested sample indicated formation of product 28b (observed mass 24446 Da, calculated mass of 24443 Da, approximately 95% of total Fc/2 fragment), resulting from transfer of 21b to core GlcNAc(Fuc)-substituted trastuzumab heavy chain.

### Example 22

### Conjugation of Trast-(GalNAz)₂ and Trast(F₂-GalNBAz)₂ 30 with BCN-PEG₂₀₀₀ at Variable Concentrations of BCN-PEG₂₀₀₀

Trast-(GalNAz)₂ and trast-(F₂-GalNBAz)₂ (30, prepared by transfer GalNBAz from UPD-derivative 23 to core GlcNAc(Fuc)-substituted trastuzumab), at a concentration of 10 μM IgG in PBS was incubated overnight at room temperature with 0 to 20 equivalents of BCN-PEG₂₀₀₀ (0 to 200 μM). Reaction products were separated by reducing SDS-PAGE followed by coomassie staining.

FIG. 10 shows the heavy chain of trastuzumab (trast-(GalNAz)₂) and 30 (trast-(F₂-GalNBAz)₂) before conjugation to BCN-PEG₂₀₀₀ (lower band) and after conjugation to BCN-PEG₂₀₀₀ (upper band). Trast-(GalNAz)₂ shows less than 50% conversion when incubated with 20 equivalents BCN-PEG₂₀₀₀ (upper panel, lane 9) while trast-(F₂-GalNBAz)₂ shows approximately 50% conversion when incubated with only 4 equivalents BCN-PEG₂₀₀₀ (lower panel, lane 4).

1-19. (canceled)

20. A process for attaching an N-acetylgalactosamine-(hetero)aryl moiety to an N-acetylglucosamine moiety, the process comprising contacting the N-acetylgalactosamine-(hetero)aryl moiety with the N-acetylglucosamine moiety in the presence of a mutant galactosyltransferase;
wherein the N-acetylglucosamine moiety is according to Formula (1): 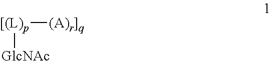
wherein:
p is 0 or 1;
q is 0 or 1;
r is 1, 2, 3 or 4;
with the proviso that when q is 1 and p is 0, then r is 1;
L is a linker;
A is independently selected from the group consisting of D, E or Q, wherein D, E and Q are as defined below;
D is a molecule of interest;
E is a solid surface; and
Q is a functional group;
and wherein the N-acetylgalactosamine-(hetero)aryl moiety is according to Formula (2): 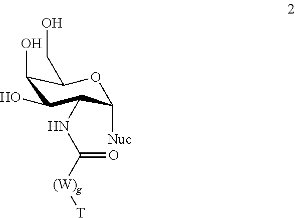
wherein:
g is 0 or 1;
T is a (hetero)aryl group, wherein the (hetero)aryl group is optionally substituted;
Nuc is a nucleotide; and
W is selected from the group consisting of C₁-C₂₄ alkylene groups, C₂-C₂₄ alkenylene groups, C₃-C₂₄ cycloalkylene groups, C₂-C₂₄ (hetero)arylene groups, C₃-C₂₄ alkyl(hetero)arylene groups and C₃-C₂₄ (hetero)arylalkylene groups, wherein the alkylene groups, alkenylene groups, cycloalkylene groups, (hetero)arylene groups, alkyl(hetero)arylene groups and (hetero)arylalkylene groups are optionally substituted, and wherein the alkylene groups, alkenylene groups, cycloalkylene groups, (hetero)arylene groups, alkyl(hetero)arylene groups and (hetero)arylalkylene groups are optionally interrupted by one or more heteroatoms selected from the group consisting of O, S and N.

21. The process according to claim 20, wherein the molecule of interest is selected from the group consisting of a reporter molecule, a diagnostic compound, an active substance, an enzyme, an amino acid, a (non-catalytic) protein, a peptide, a polypeptide, an oligonucleotide, a monosaccharide, an oligosaccharide, a polysaccharide, a glycan, a (poly)ethylene glycol diamine, a polyethylene glycol chain, a polyethylene oxide chain, a polypropylene glycol chain, a polypropylene oxide chain and a 1,x-diaminoalkane, wherein x is the number of carbon atoms in the alkane.

22. The process according to claim 20, wherein the solid surface is selected from the group consisting of functional surfaces, nanomaterials, carbon nanotubes, fullerenes, virus capsids, metal surfaces, metal alloy surfaces and polymer surfaces.

23. The process according to claim 20, wherein Q is a functional group selected from the group consisting of hydrogen, halogen, R³, —CH═C(R³)₂, —C≡CR³, —[C(R³)₂C(R³)₂O]_{q}—R³ wherein q is in the range of 1 to 200, —CN, —N₃, —NCX, —XCN, —XR³, —N(R³)₂, —⁺N(R³)₃, —C(X)N(R³)₂, —C(R³)₂XR³, —C(X)R³, —C(X)XR³, —S(O)R³, —S(O)₂R³, —S(O)OR³, —S(O)₂OR³, —S(O)N(R³)₂, —S(O)₂N(R³)₂, —OS(O)R³, —OS(O)₂R³, —OS(O)OR³, —OS(O)₂OR³, —P(O)(R³)(OR³), —P(O)(OR³)₂, —OP(O)(OR³)₂, —Si(R³)₃, —XC(X)R³, —XC(X)X³, —X C(X)N(R³)₂, —N(R³)C(X)R³, —N(R³)C(X)XR³ and —N(R³)C(X)N(R³)₂, wherein X is oxygen or sulphur and wherein R³ is independently selected from the group consisting of hydrogen, halogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups optionally substituted and optionally interrupted by one or more heteroatoms selected from O and N.

24. The process according to claim 20, wherein the mutant galactosyltransferase is selected from the group consisting of mutant β(1,4)-galactosyltransferases and mutant β(1,3)-N-galactosyltransferases.

25. The process according to claim 20, wherein the mutant galactosyltransferase is selected from the group consisting of bovine or human β(1,4)-Gal-T1 GalT Y289L, GalT Y289N, GalT Y289I, Y289F, GalT Y289M, GalT Y289V, GalT Y289G, GalT Y289I and GalT Y289A.

26. The process according to claim 20, wherein the mutant galactosyltransferase is selected from the group consisting of bovine or human β(1,4)-Gal-T1 GalT Y289L C342T, GalT Y289N C342T, GalT Y289I C342T, Y289F C342T, GalT Y289M C342T, GalT Y289V C342T, GalT Y289G C342T, GalT Y289I C342T and GalT Y289A C342T.

27. The process according to claim 20, wherein the N-acetylgalactosamine-(hetero)aryl moiety is according to Formula (3b): 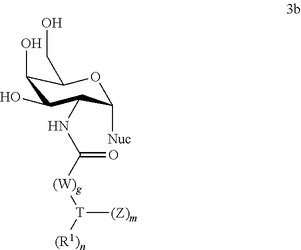
wherein g, T, Nuc and W are as defined in claim 20;
m is 0-8;
n is 0-8;
Z is independently selected from the group of functional groups; and
R¹ is independently selected from the group consisting of C₁-C₂₄ alkyl groups, C₂-C₂₄ (hetero)aryl groups, C₃-C₂₄ alkyl(hetero)aryl groups, C₃-C₂₄ (hetero)arylalkyl groups, C₂-C₂₄ alkenyl groups, C₂ C₂₄ alkynyl groups, C₃-C₂₄ cycloalkyl groups, C₅ C₂₄ cycloalkenyl groups, C₈-C₂₄ cycloalkynyl groups, C₁-C₂₄ alkoxy groups, C₂ C₂₄ alkenyloxy groups, C₂-C₂₄ (hetero)aryloxy groups, C₃-C₂₄ alkyl(hetero)aryl groups, C₃-C₂₄ (hetero)arylalkyl groups, C₂-C₂₄ alkynyloxy groups and C₃-C₂₄ cycloalkyloxy groups, wherein the alkyl groups, (hetero)aryl groups, alkyl(hetero)aryl groups, (hetero)arylalkyl groups, alkenyl groups, alkynyl groups, cycloalkyl groups, alkoxy groups, alkenyloxy groups, (hetero)aryloxy groups, alkynyloxy groups and cycloalkyloxy groups are optionally substituted, the alkyl groups, the alkoxy groups, the cycloalkyl groups and the cycloalkoxy groups being optionally interrupted by one of more hetero-atoms selected from the group consisting of O, N and S.

28. The process according to claim 20, wherein the N-acetylgalactosamine-(hetero)aryl moiety is according to Formula (5a), (5b), (5c), (5d), (5e) or (5f): 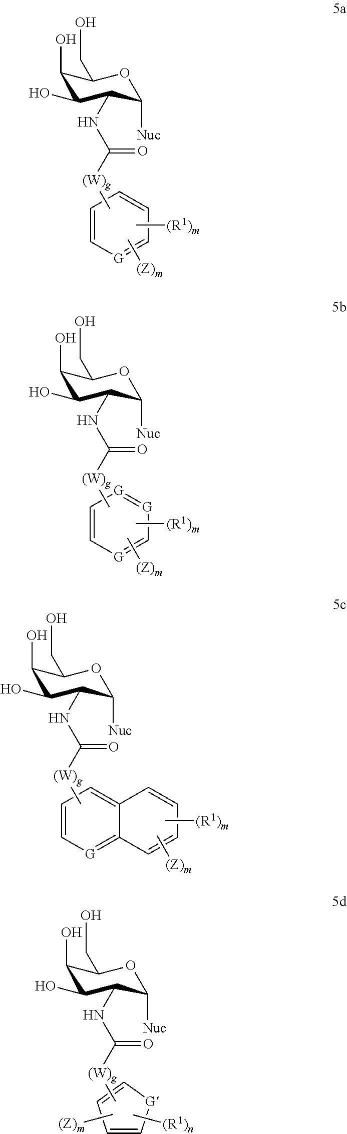 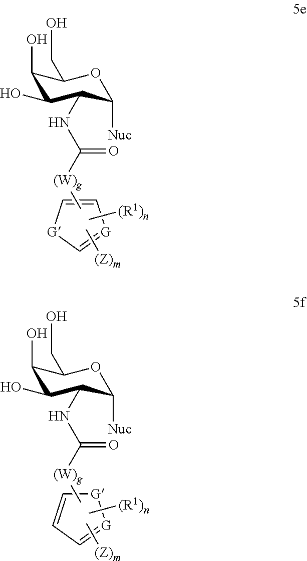
wherein:
Nuc, W and g are as defined in claim 20;
R¹ Z, m and n are as defined in claim 24;
G is independently selected from the group consisting of N, CR⁴, CR⁵, CZ and N⁺R⁴, wherein R⁴ is selected from the group consisting of C₁-C₂₄ alkyl groups, and R⁵ is selected from the group consisting of hydrogen, R¹ and R⁴, and wherein R¹ is as defined in claim 24; and
G′ is selected from the group consisting of O, S, NR⁵ and N⁺(R⁴)₂, wherein R⁴ and R⁵ are as defined above.

29. The process according to claim 30, wherein the N-acetylgalactosamine-(hetero)aryl moiety is according to Formula (23b): 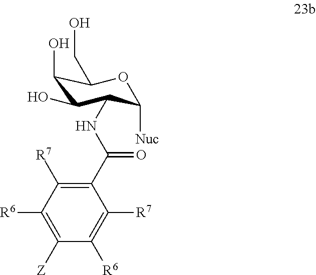
wherein:
Nuc is a nucleotide;
Z is a functional group;
R⁶ is independently selected from the group consisting of hydrogen, F, Cl, Br and I; and
R⁷ is independently selected from the group consisting of hydrogen, F, Cl, Br and I.

30. The process according to claim 20, wherein Z is independently selected from the group consisting of a 1,3-dipole functional group, halogen, R³, —CH═C(R³)₂, —C≡CR³, —[C(R³)₂C(R³)₂O]_{q}—R³ wherein q is in the range of 1 to 200, —CN, —N₃, —NCX, —XCN, —XR³, —N(R³)₂, —⁺N(R³)₃, —C(X)N(R³)₂, —C(R³)₂XR³, —C(X)R³, —C(X)XR³, —S(O) R³, —S(O)₂R³, —S(O)OR³, —S(O)₂OR³, —S(O)N(R³)₂, —S(O)₂N(R³)₂, —OS(O)R³, —OS(O)₂R³, —OS (O)OR³, —OS(O)₂OR³, —P(O)(R³)(OR³), —P(O)(OR³)₂, —OP(O)(OR³)₂, —Si(R³)₃, —XC(X)R³, —X C(X)XR³, —XC(X)N(R³)₂, —N(R³)C(X)R³, —N(R³)C(X)XR³ and N(R³)C(X)N(R³)₂, wherein X is oxygen or sulphur and wherein R³ is independently selected from the group consisting of hydrogen, halogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups optionally substituted and optionally interrupted by one or more heteroatoms selected from O and N.

31. The process according to claim 20, wherein the N-acetylglucosamine moiety is a terminal GlcNAc moiety of a glycoprotein glycan.

32. The process according to claim 20, wherein the N-acetylglucosamine moiety is a glycoprotein according to Formula (10) or (11): 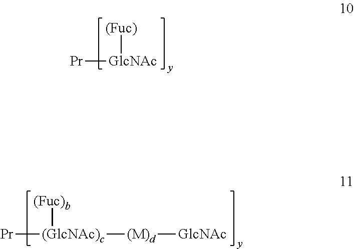
wherein:
y is 1-20;
b is 0 or 1;
c is 0 or 1;
d is 0 or 1;
Pr is a glycoprotein; and
M is a monosaccharide, or a linear or branched oligosaccharide comprising 2 to 20 saccharide moieties.

33. A glycoprotein according to Formula (8) or (9): 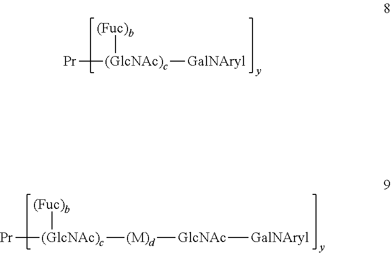
wherein:
y is 1-20;
b is 0 or 1;
c is 0 or 1;
d is 0 or 1;
Pr is a glycoprotein; and
M is a monosaccharide, or a linear or branched oligosaccharide comprising 2 to 20 saccharide moieties; and
wherein GalNAryl is according to Formula (6): 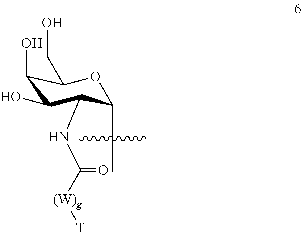
wherein:
W, T and g are as defined in claim 20; and
T is optionally substituted.

34. The glycoprotein according to claim 33, wherein GalNAryl is according to Formula (7): 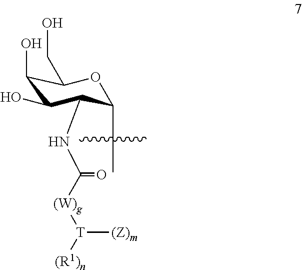
wherein:
T, W and g are as defined in claim 20; and
R¹, Z, n and m are as defined in claim 24.

35. The glycoprotein according to claim 33, wherein GalNAryl is according to Formula (23f), (21f) or (21g): 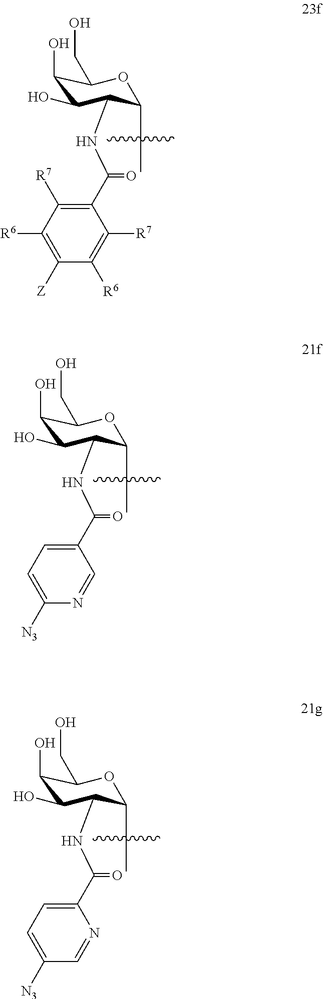
wherein:
Z is a functional group;
R⁶ is independently selected from the group consisting of hydrogen, F, Cl, Br and I; and
R⁷ is independently selected from the group consisting of hydrogen, F, Cl, Br and I.

36. A compound according to formula (3b): 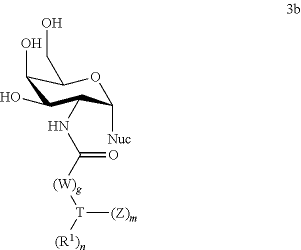
wherein:
Nuc, W and T are as defined in claim 20;
Z and R¹ are as defined in claim 24;
g is 0;
m is 0, 1, 2, 3, 4, 5, 6, 7 or 8; and
n is 0, 1, 2, 3, 4, 5, 6, 7 or 8.

37. The compound according to claim 36, wherein the compound is according to Formula (23b): 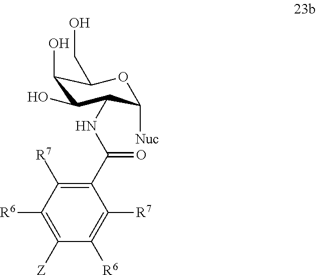
wherein:
Nuc is a nucleotide;
Z is a functional group;
R⁶ is independently selected from the group consisting of hydrogen, F, Cl, Br and I; and
R⁷ is independently selected from the group consisting of hydrogen, F, Cl, Br and I.

38. The compound according to claim 36, wherein the compound is according to Formula (23), (23c), (23d) or (23e): 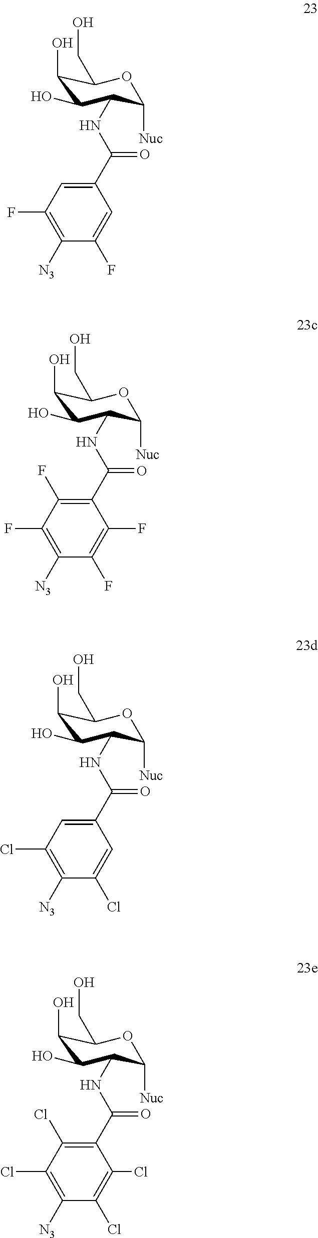
wherein:
Nuc is a nucleotide.

39. The compound according to claim 36, wherein the compound is according to Formula (21b) or (21), or according to Formula (21c), (21d) or (21e): 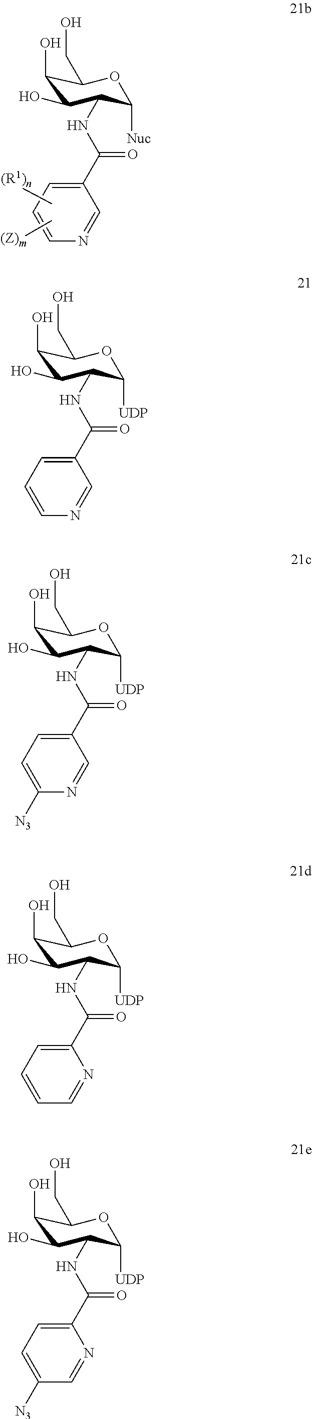
wherein:
Nuc is a nucleotide; and
Z, R¹, m and n are as defined in claim 24.

40. The compound according to claim 36, wherein the compound is according to Formula (22b) or (22): 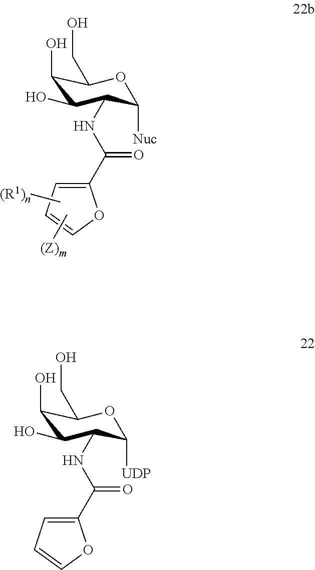
wherein:
Nuc is a nucleotide; and
Z, R¹, m and n are as defined in claim 24.

41. The compound according to claim 36, wherein Nuc is UDP.
